(19) United States
(12) Patent Application Publication
(10) Pub. No.: US 2009/0286777 A1
(43) Pub. Date: Nov. 19, 2009

(54) 5,6,Fused Pyrrolidine Compounds Useful as Tachykinin Receptor Antagonists

(76) Inventors: Jianming Bao, Scotch Plains, NJ (US); Robert J. DeVita, Westfied, NJ (US); Sander G. Mills, Scotch Plains, NJ (US); Gregori J. Morriello, Randolph, NJ (US)

Correspondence Address: MERCK AND CO., INC, P O BOX 2000, RAHWAY, NJ, 07065-0907, US

(21) Appl. No.: 12/226,294

(22) PCT Filed: May 11, 2007
(86) PCT No.: PCT/US07/11367

§ 371 Date: Oct. 14, 2008

Related U.S. Application Data
(60) Provisional application No. 60/800,477, filed on May 15, 2006.

(51) Int. Cl. A61K 31/437; C07D 471/04; A61K 31/5377; A61K 31/506

(52) U.S. Cl. 514/233.2; 546/183; 544/127; 544/326; 514/299; 514/256

(57) ABSTRACT

The present invention is directed to certain 5,6-fused pyrrolidine compounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### BACKGROUND OF THE INVENTION

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to substance P, the known mammalian tachykinins include neurokinin A and neurokinin B. The current nomenclature designates the receptors for substance P, neurokinin A, and neurokinin B as neurokinin-1 (NK-1), neurokinin-2 (NK-2), and neurokinin-3 (NK-3), respectively.

Tachykinin, and in particular substance P, antagonists are useful in the treatment of clinical conditions which are characterized by the presence of an excess of tachykinin, in particular substance P, activity, including disorders of the central nervous system, nociception and pain, gastrointestinal disorders, disorders of bladder function and respiratory diseases.

### SUMMARY OF THE INVENTION

The present invention is directed to certain 5,6-fused pyrrolidine compounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### DETAILED DESCRIPTION OF THE INVENTION

The present invention is directed to compounds of formula I:

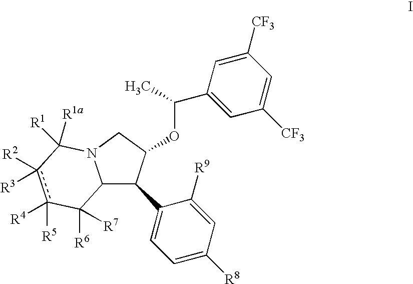

wherein:
R¹ and R^{1a} are each hydrogen or together with the carbon atom to which they are attached form a carbonyl;
R² is selected from the group consisting of:
(1) hydrogen, and
(2) CH₃;
   R³ are each independently selected from the group consisting of:
(1) hydrogen,
(2) hydroxyl,
(3) NH₂,
(4) C₁₋₆alkyl,
(5) hydroxyC₁₋₆alkyl,
(6) C₁₋₆alkyl-O—C₁₋₆alkyl,
(7) N(CH₃)₂;
(8) NH—C(O)—C(CH₃)₂—NH₂,
(9) NH—C(O)—CF₃,
(10) -A1, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocycle is optionally substituted with a group selected from methyl, oxo and hydroxyl,
(11) —NH-A1
(12) —NH—CH₂-A1,
(13) —O—C(O)—CH₃,
(14) CO₂Me; and
(15) CO₂H;
   R⁴ is selected from hydrogen and methyl;
   R⁵ is selected from a group consisting of:
(1) hydrogen,
(2) hydroxy,
(3) hydroxyC₁₋₃alkyl,
(4) C₁₋₄alkyl,
(5) C₁₋₄alkyl-NH₂,
(6) C₁₋₄alkyl-NH—C(O)CH₃,
(7) C₁₋₄alkyl-NH—C(O)CH₂Cl,
(8) C₁₋₄alkyl-NH—C(O)H,
(9) —C(O)—O—CH₃,
(10) —C(O)—CH₃,
(11) —O—S(O)₂—CF₃,
(12) —C(O)—OH,
(13) —C(O)—N(R¹⁰)(R¹¹),
(14) phenyl, optionally substituted with a substituent selected from the group consisting of halo, methyl, hydroxyC₁₋₄alkyl, —C(O)H and —NH—S(O)₂—CH₃,
(15) —NH—S(O)₂—CH₃,
(16) —NH-cyclopentenone,
(17) —NH-cyclohexenone, optionally substituted with a substituent selected from halo, hydroxy and oxo,
(18) —NH—C(O)—C₁₋₄alkyl,
(19) —NH—C(O)-phenyl, optionally substituted with halo, —C(O)H or cyano,
(20) —NH—C(O)-pyridyl, optionally substituted with halo, —C(O)H or cyano,
(21) —NH—C(O)—NH-phenyl, optionally substituted with halo, —C(O)H or cyano,
(22) —NH—C(O)—NH-pyridyl, optionally substituted with halo, —C(O)H or cyano,
(23) —NH—C(O)—O—CH₂—C₂₋₄alkenyl,
(24) —NH—C₁₋₄alkyl,
(25) —NH—C(O)—C₁₋₄alkyl-NH₂,
(26) —NH—C(O)—C₁₋₄alkyl-NH₂—C(O)—O—CH₂-phenyl,
(27) —N(R¹⁰)(R¹¹),
(28) A², wherein A² is selected from the group consisting of

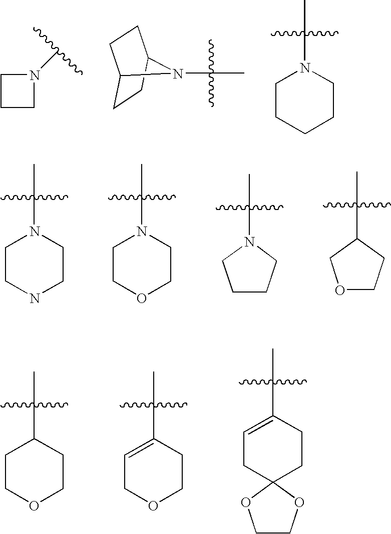

wherein A2 is optionally substituted with one or two groups selected from halo, hydroxyl, cyano, C₁₋₄alkyl, NH₂, COOH, oxo, —COO—C₁₋₄alkyl, —NH—C(O)—CH₂Cl, —C(O)CH₃, and

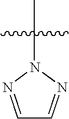

(29) A3, wherein A3 is a heteraromatic ring of 5 or 6 atoms or N-oxide thereof, wherein 1, 2, or 3 of the atoms is a heteroatom selected from N, S or O, and wherein at least one of the heteroatoms is a N, and wherein the heteroaryl or heterocycle is optionally substituted with one or two groups selected from halo, cyano, C₁₋₄alkyl, oxo, hydroxyl, phenyl, benzyl, —OCH₃, —CH₃—NH₂, —NH—C(O)—CH₃Cl, and —CH₃—N(CH₃)₂,
(30) CH₃-A2,
(31) CH₃-A3,
(32) —NH-A2,
(33) —C(O)-A2,
(34) —NH-A3,
(35) —C(O)-A3,
or R⁴ and R⁵ together with the carbon to which they are attached form a carbonyl;
   R⁶ is selected from hydrogen and methyl,
   R⁷ are each independently selected from a group consisting of:
(1) hydrogen,
(2) halo, and
(3) hydroxyl, and
(4) methyl,
(5) —C(O)OH,
(6) —O—C(O)—CH₃,
(7) NH—C(O)—CH₃,
(8) NH—C(O)-phenyl,
(9) NH—C(O)—O—CH₃,
(10) NH₂, and
(11) A4, wherein A4 is a heteroaryl or N-oxide thereof or heterocycle of 5 or 6 atoms wherein the heteroaryl or heterocyle contains 1, 2 or 3 heteroatoms selected from the group consisting of O, N and S, and wherein the heteroaryl or heterocycle is optionally substituted with a group selected from methyl, oxo, hydroxyl, —CH₃—NH₂ and —CH₃—N(CH₃)₂;
or R⁶ and R⁷ together with the carbon to which they are attached form a carbonyl;
   R⁸ and R⁹ are each independently selected from a group consisting of:
(1) hydrogen,
(2) halo, and
(3) methyl;
   R¹⁰ ad R¹¹ are each selected from the group consisting of
(1) hydrogen,
(2) methyl,
(3) —O—CH₃
(4) A5, wherein A5 is a heteroaryl or N-oxide thereof or heterocycle of 5 or 6 atoms wherein the heteroaryl or heterocyle contains 1, 2 or 3 heteroatoms selected from the group consisting of O, N and S, and wherein the heteroaryl or heterocycle is optionally substituted with a group selected from methyl, oxo, hydroxyl, —CH₃—NH₂ and —CH₃—N(CH₃)₂, and
(5) —C₁₋₃alkyl-A5,
   or a pharmaceutically acceptable salts thereof and individual enantiomers and diastereomers thereof.

Within this aspect there is a genus of compounds wherein

A1 is selected from the group consisting of

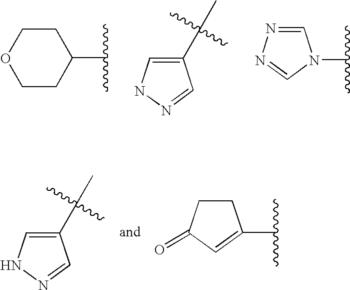

Within this aspect there is a genus of compounds wherein
R³ is selected from the group consisting of
(1) NH₂, and
(2) —NH-A1, wherein A1 is selected from the group consisting of

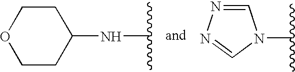

and A1 is optionally substituted with a group selected from methyl, oxo and hydroxyl;

Within this aspect there is a genus of compounds wherein A3 is selected from the group consisting of

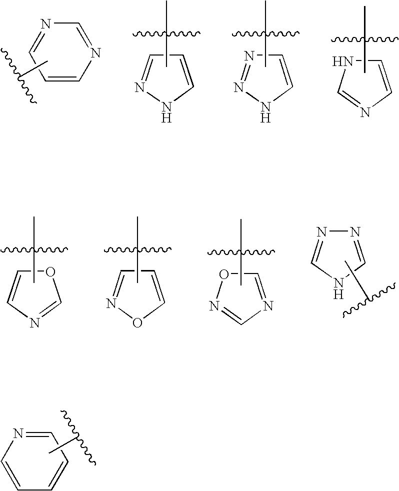

or an N-oxide thereof wherein A3 is optionally substituted with one or two substituents selected from the group consisting of halo, cyano, C₁₋₄alkyl, oxo, hydroxyl, phenyl, benzyl, —OCH₃, —CH₃—NH₂, —NH—C(O)—CH₃Cl, and —CH₃—N(CH₃)₂.

Within this aspect there is a genus of compounds wherein

A compound according to claim 1 wherein A4 is selected from the group consisting of

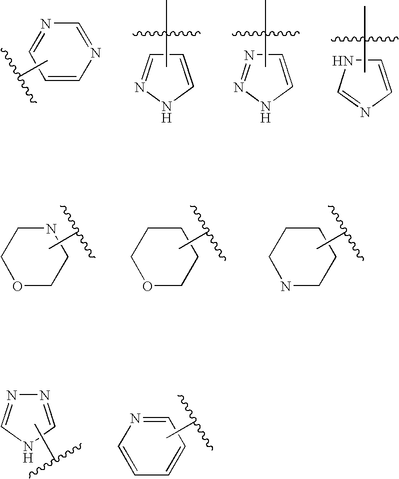

wherein A4 is optionally substituted with one or two substituents selected from the group consisting of hydroxylmethyl, oxo, hydroxyl, —CH₃—NH₂ and —CH₃—N(CH₃)₂.

Within this aspect there is a genus of compounds wherein

R⁵ is selected from a group consisting of:
(1) hydroxy,
(2) NH₂, and
(3) N(R¹⁰)(R¹¹).

Within this aspect there is a genus of compounds wherein

R⁷ is selected from a group consisting of:
(1) hydrogen,
(2) fluoro, and
(3) methyl.

Within this aspect there is a genus of compounds wherein

R⁸ and R⁹ are each independently selected from a group consisting of:
(1) hydrogen,
(2) fluoro, and
(3) methyl.

Within this aspect there is a genus of compounds wherein

R⁶ is fluoro and R⁷ is methyl.

Within this aspect there is a genus of compounds wherein

R⁸ is fluoro and R⁹ is methyl or hydrogen.

Within this aspect there is a genus of the formula

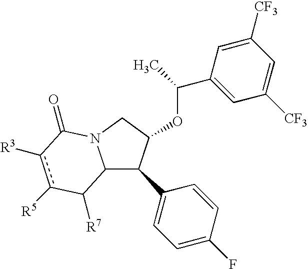

Within this aspect there is a genus of compounds wherein

R¹ and R^{1a} are each hydrogen or together with the carbon atom to which they are attached form a carbonyl;
R² is selected from the group consisting of:
(1) hydrogen, and
(2) CH₃;
   R³ is selected from the group consisting of
(1) NH₂, and
(2) —NH-A1, wherein A1 is selected from the group consisting of

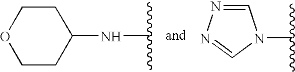

and A1 is optionally substituted with a group selected from methyl, oxo and hydroxyl;
   R⁴ is selected from hydrogen and methyl;
   R⁵ is selected from a group consisting of:
(1) hydroxy,
(2) NH₂, and
(3) N(R¹⁰)(R¹¹);
   R⁶ is selected from hydrogen and methyl,
   R⁷ is selected from a group consisting of:
(1) hydrogen,
(2) fluoro, and
(3) methyl.
   R⁸ is fluoro and R⁹ is methyl or hydrogen.
   R¹⁰ ad R¹¹ are each selected from the group consisting of
(1) hydrogen, and
(2) -A4, wherein A4 is selected from the group consisting of

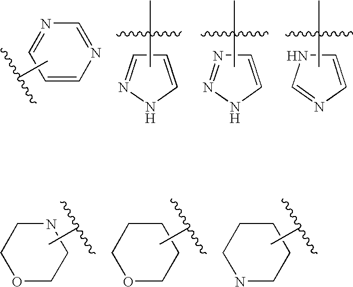

wherein A4 is optionally substituted with a substituent selected from the group consisting of hydroxyl, oxo, methyl, —CH₃—NH₂ and —CH₃—N(CH₃)₂;
or a pharmaceutically acceptable salts thereof and individual enantiomers and diastereomers thereof.
or a pharmaceutically acceptable salts thereof and individual enantiomers and diastereomers thereof.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “aryl” unless specifically stated otherwise includes multiple ring systems as well as single ring systems such as, for example, phenyl or naphthyl.

The term heteroaryl include, for example, pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl, tetrazolyl.

The term heterocycle includes, for example, azetidinyl, pyrrolidinyl, piperidinyl, piperazinyl, morpholinyl, tetrahydrofuranyl, imidazolinyl, and thiomorpholinyl.

Specific embodiments of the present invention include a compound which is selected from the group consisting of the subject compounds of the Examples herein and pharmaceutically acceptable salts thereof and individual enantiomers and diastereomers thereof.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

There are several acceptable methods of naming the compounds discussed herein. There are several acceptable methods of naming the compounds discussed herein.

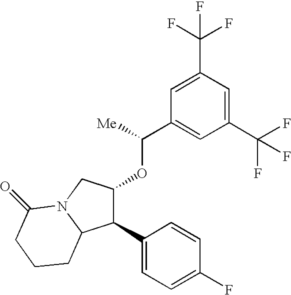

For example, the above compound can be named (1S,2R)-2-{(1R)-1-[3,5-bis(trifluoromethyl)phenyl]ethoxy}-1-(4-fluorophenyl)hexahydroindolizin-5(1H)-one. The core structure may be generally referred to as hexahydroindolizin-5(1H)-one, hexahydroindolizinone perhydroindolizin-5(1H)-one, perhydroindolizinone and indolizinone compounds.

For example, the above compound can be named “(6R,7S,7aS)-6-{(1R)-1-[3,5-bis(trifluoromethyl)phenyl]ethoxy}-7-(4-fluorophenyl)hexahydro-3H-pyrrolizin-3-one. The core structure may be generally referred to as hexahydro-3H-pyrrolizin-3-one, hexahydropyrrolizinone perhydro-3H-pyrrolizin-3-one, perhydropyrrolizinone compounds.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₈alkyl specifically includes methyl, ethyl, n-propyl, iso-propyl, n-butyl, iso-butyl, tert-butyl, pentyl, and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of the present invention are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The compounds of the present invention are useful in the prevention and treatment of a wide variety of clinical conditions which are characterized by the presence of an excess of tachykinin, in particular substance P, activity. Thus, for example, an excess of tachykinin, and in particular substance P, activity is implicated in a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delerium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple aetiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremour; substance-related disorders arising from the use of alcohol, amphetamines (or amphetamine-like substances) caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delerium, withdrawal delerium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid haemorrhage or cerebral oedema.

Tachykinin, and in particular substance P, activity is also involved in nociception and pain. The compounds of the present invention will therefore be of use in the prevention or treatment of diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhoea, and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; ankylosing spondylitis, gout; and scar pain.

Tachykinin, and in particular substance P, antagonists may also be of use in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis. Tachykinin, and in particular substance P, antagonists may also be of use in the treatment of neoplasms, including breast tumours, neuroganglioblastomas and small cell carcinomas such as small cell lung cancer.

Tachykinin, and in particular substance P, antagonists may also be of use in the treatment of gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-oesophageal reflux disease, acid indigestion, over indulgence in food or drink, acid stomach, waterbrash or regurgitation, heartburn, for example, episodic, nocturnal or meal-induced heartburn, and dyspepsia.

Tachykinin, and in particular substance P, antagonists may also be of use in the treatment of a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia, frequent urination and urinary incontinence, including the prevention or treatment of overactive bladder with symptoms of urge urinary incontinence, urgency, and frequency; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially the transmission of pain in migraine. The compounds of the present invention are also of value in the treatment of a combination of the above conditions, in particular in the treatment of combined post-operative pain and post-operative nausea and vomiting.

The compounds of the present invention are particularly useful in the prevention or treatment of emesis, including acute, delayed or anticipatory emesis, such as emesis induced by chemotherapy, radiation, toxins, pregnancy, vestibular disorders, motion, surgery, migraine, and variations in intercranial pressure. For example, the compounds of the present invention are of use optionally in combination with other antiemetic agents for the prevention of acute and delayed nausea and vomiting associated with initial and repeat courses of moderate or highly emetogenic cancer chemotherapy, including high-dose cisplatin. Most especially, the compounds of the present invention are of use in the treatment of emesis induced by antineoplastic (cytotoxic) agents, including those routinely used in cancer chemotherapy, and emesis induced by other pharmacological agents, for example, rolipram. Examples of such chemotherapeutic agents include alkylating agents, for example, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for instance, by D. J. Stewart in Nausea and Vomiting: Recent Research and Clinical Advances, Eds. J. Kucharczyk et al, CRC Press Inc., Boca Raton, Fla., USA (1991) pages 177-203, especially page 188. Commonly used chemotherapeutic agents include cisplatin, dacarbazine (DTIC), dactinomycin, mechlorethamine, streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil [R. J. Gralla et al in Cancer Treatment Reports (1984) 68(1), 163-172]. A further aspect of the present invention comprises the use of a compound of the present invention for achieving a chronobiologic (circadian rhythm phase-shifting) effect and alleviating circadian rhythm disorders in a mammal. The present invention is further directed to the use of a compound of the present invention for blocking the phase-shifting effects of light in a mammal.

The present invention is further directed to the use of a compound of the present invention or a pharmaceutically acceptable salt thereof, for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal which comprising the administration of a compound of the present invention or a pharmaceutically acceptable salt thereof. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during withdrawal stages), childhood onset DIMS, nocturnal myoclonus, fibromyalgia, muscle pain, sleep apnea and restless legs and non specific REM disturbances as seen in ageing.

The particularly preferred embodiments of the instant invention are the treatment of emesis, urinary incontinence, depression or anxiety by administration of the compounds of the present invention to a subject (human or animal) in need of such treatment.

The present invention is directed to a method for the manufacture of a medicament for antagonizing the effect of substance P at its receptor site or for the blockade of neurokinin-1 receptors in a mammal comprising combining a compound of the present invention with a pharmaceutical carrier or diluent. The present invention is further directed to a method for the manufacture of a medicament for the treatment of a physiological disorder associated with an excess of tachykinins in a mammal comprising combining a compound of the present invention with a pharmaceutical carrier or diluent.

The present invention also provides a method for the treatment or prevention of physiological disorders associated with an excess of tachykinins, especially substance P, which method comprises administration to a patient in need thereof of a tachykinin reducing amount of a compound of the present invention or a composition comprising a compound of the present invention. As used herein, the term “treatment” or “to treat” refers to the administration of the compounds of the present invention to reduce, ameliorate, or eliminate either the symptoms or underlying cause of the noted disease conditions, in a subject (human or animal) that suffers from that condition or displays clinical indicators thereof. The term “prevention” or “to prevent” refers to the administration of the compounds of the present invention to reduce, ameliorate, or eliminate the risk or likelihood of occurrence of the noted disease conditions, in a subject (human or animal) susceptible or predisposed to that condition.

The compounds of this invention are useful for antagonizing tachykinins, in particular substance P in the treatment of gastrointestinal disorders, central nervous system disorders, inflammatory diseases, pain or migraine and asthma in a mammal in need of such treatment. This activity can be demonstrated by the following assays.

Receptor Expression in COS: To express the cloned human neurokinin-1 receptor (NK1R) transiently in COS, the cDNA for the human NK1R was cloned into the expression vector pCDM9 which was derived from pCDM8 (INVITROGEN) by inserting the ampicillin resistance gene (nucleotide 1973 to 2964 from BLUESCRIPT SK+) into the Sac II site. Transfection of 20 ug of the plasmid DNA into 10 million COS cells was achieved by electroporation in 800 ul of transfection buffer (135 mM NaCl, 1.2 mM CaCl₂, 1.2 mM MgCl₂, 2.4 mM K₂HPO₄, 0.6 mM KH₂PO₄, 10 mM glucose, 10 mM HEPES pH 7.4) at 260 V and 950 uF using the IBI GENEZAPPER (IBI, New Haven, Conn.). The cells were incubated in 10% fetal calf serum, 2 mM glutamine, 100 U/ml penicillin-streptomycin, and 90% DMEM media (GIBCO, Grand Island, N.Y.) in 5% CO₂ at 37° C. for three days before the assay.

Stable Expression in CHO: To establish a stable cell line expressing the cloned human NK1R, the cDNA was subcloned into the vector pRcCMV (INVITROGEN). Transfection of 20 ug of the plasmid DNA into CHO cells was achieved by electroporation in 800 ul of transfection buffer suplemented with 0.625 mg/ml Herring sperm DNA at 300 V and 950 uF using the IBI GENEZAPPER (IBI). The transfected cells were incubated in CHO media [10% fetal calf serum, 100 U/ml penicillin-streptomycin, 2 mM glutamine, 1/500 hypoxanthine-thymidine (ATCC), 90% IMDM media (JRH BIOSCIENCES, Lenexa, Kans.), 0.7 mg/ml G418 (GIBCO)] in 5% CO₂ at 37° C. until colonies were visible. Each colony was separated and propagated. The cell clone with the highest number of human NK1R was selected for subsequent applications such as drug screening.

Assay Protocol using COS or CHO: The binding assay of human NK1R expressed in either COS or CHO cells is based on the use of ¹²⁵I-substance P (¹²⁵I-SP, from DU PONT, Boston, Mass.) as a radioactively labeled ligand which competes with unlabeled substance P or any other ligand for binding to the human NKIR. Monolayer cell cultures of COS or CHO were dissociated by the non-enzymatic solution (SPECIALTY MEDIA, Lavallette, N.J.) and resuspended in appropriate volume of the binding buffer (50 mM Tris pH 7.5, 5 mM MnCl₂, 150 mM NaCl, 0.04 mg/ml bacitracin, 0.004 mg/ml leupeptin, 0.2 mg/ml BSA, 0.01 mM phosphoramidon) such that 200 ul of the cell suspension would give rise to about 10,000 cpm of specific ¹²⁵I-SP binding (approximately 50,000 to 200,000 cells). In the binding assay, 200 ul of cells were added to a tube containing 20 ul of 1.5 to 2.5 nM of ¹²⁵I-SP and 20 ul of unlabeled substance P or any other test compound. The tubes were incubated at 4° C. or at room temperature for 1 hour with gentle shaking. The bound radioactivity was separated from unbound radioactivity by GF/C filter (BRANDEL, Gaithersburg, Md.) which was pre-wetted with 0.1% polyethylenimine. The filter was washed with 3 ml of wash buffer (50 mM Tris pH 7.5, 5 mM MnCl₂, 150 mM NaCl) three times and its radioactivity was determined by gamma counter. The activation of phospholipase C by NK1R may also be measured in CHO cells expressing the human NK1R by determining the accumulation of inositol monophosphate which is a degradation product of IP₃. CHO cells are seeded in 12-well plate at 250,000 cells per well. After incubating in CHO media for 4 days, cells are loaded with 0.025 uCi/ml of ³H-myoinositol by overnight incubation. The extracellular radioactivity is removed by washing with phosphate buffered saline. LiCl is added to the well at final concentration of 0.1 mM with or without the test compound, and incubation is continued at 37° C. for 15 min. Substance P is added to the well at final concentration of 0.3 nM to activate the human NKIR. After 30 min of incubation at 37° C., the media is removed and 0.1 N HCl is added. Each well is sonicated at 4° C. and extracted with CHCl₃/methanol (1:1). The aqueous phase is applied to a 1 ml Dowex AG 1×8 ion exchange column. The column is washed with 0.1 N formic acid followed by 0.025 M ammonium formate-0.1 N formic acid. The inositol monophosphate is eluted with 0.2 M ammonium formate-0.1 N formic acid and quantitated by beta counter. In particular, the intrinsic tachykinin receptor antagonist activities of the compounds of the present invention may be demonstrated by these assays. The compounds of the following examples have activity in the aforementioned assays in the range of 0.05 nM to 10 μM. The activity of the present compounds may also be demonstrated by the assay disclosed by Lei, et al., British J. Pharmacol., 105, 261-262 (1992).

According to a further or alternative aspect, the present invention provides a compound of the present invention for use as a composition that may be administered to a subject in need of a reduction of the amount of tachykinin or substance P in their body.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The compositions containing compounds of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples in the pharmacy arts of unit dosage forms. The compositions containing compounds of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention to the individual in need of treatment in a form that can be introduced into that individuals body in a therapeutically useful form and therapeutically effective amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories. The term “therapeutically effective amount” refers to a sufficient quantity of the compounds of the present invention, in a suitable composition, and in a suitable dosage form to treat or prevent the noted disease conditions.

The compounds of the present invention may be administered in combination with another substance that has a complimentary effect to the tachykinin and substance P inhibitors of the present invention. Accordingly, in the prevention or treatment of emesis, a compound of the present invention may be used in conjunction with other anti-emetic agents, especially SHT3 receptor antagonists, such as ondansetron, granisetron, tropisetron, palenosetron and zatisetron, a corticosteroid, such as dexamethasone, or GABA_{B} receptor agonists, such as baclofen. Likewise, for the prevention or treatment of migraine a compound of the present invention may be used in conjunction with other anti-migraine agents, such as ergotamines or 5HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, corticotropin releasing factor (CRF) antagonists, and pharmaceutically acceptable salts thereof. For the treatment or prevention of eating disorders, including obesity, bulimia nervosa and compulsive eating disorders, a compound of the present invention may be used in conjunction with other anorectic agents. It will be appreciated that for the treatment or prevention of pain or nociception or inflammatory diseases, a compound of the present invention may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent.

It will be appreciated that when using any combination described herein, both the compound of the present invention and the other active agent(s) will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, one active component may be administered as a tablet and then, within a reasonable period of time, the second active component may be administered either as an oral dosage form such as a tablet or a fast-dissolving oral dosage form. By a “fast dissolving oral formulation” is meant, an oral delivery form which when placed on the tongue of a patient, dissolves within about 10 seconds. By “reasonable period of time” is meant a time period that is not in excess of about 1 hour. That is, for example, if the first active component is provided as a tablet, then within one hour, the second active component should be administered, either in the same type of dosage form, or another dosage form which provides effective delivery of the medicament.

The compounds of this invention may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

In the treatment of the conditions associated with an excess of tachykinins, a suitable dosage level of the compounds of the present invention, or pharmaceutically acceptable salts thereof, is about 0.001 to 50 mg/kg per day, in particular about 0.01 to about 25 mg/kg, such as from about 0.05 to about 10 mg/kg per day. The dosage range will generally be about 0.5 to 1000 mg per patient per day, which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Specific dosages of the compounds of the present invention, or pharmaceutically acceptable salts thereof, for administration include 1 mg, 5 mg, 10 mg, 30 mg, 100 mg, and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions for treatment or prevention of excess tachykinins comprise about 1 mg, 5 mg, 10 mg, 30 mg, 100 mg, and 500 mg of active ingredient.

Several methods for preparing the compounds of this invention are illustrated in the following Examples. Starting materials and the requisite intermediates are in some cases commercially available, or can be prepared according to literature procedures or as illustrated herein. All ¹H NMR spectra were obtained on instrumentation at a field strength of 400 or 500 MHz.

The following examples are provided for the purpose of further illustration only and are not intended to be limitations on the disclosed invention.

### Example 1

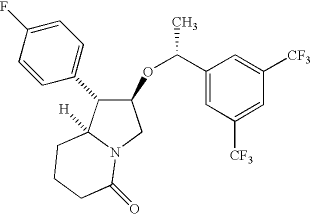

### (1S,2R,8aS)-2-{(1R)-1-[3,5-bis(trifluoromethyl)phenyl]ethoxy}-1-(4-fluorophenyl)hexahydroindolizin-5(1H)-one

### Step A: tert-butyl (2R,3S,4R)-4-{(1R)-1-[3,5-bis(trifluoromethyl)phenyl]ethoxy}-3-(4-fluorophenyl)-2-formylpyrrolidine-1-carboxylate

To a stirred solution of 1.32 mL (15.1 mmol) oxalyl chloride in 70 mL dry methylene chloride under nitrogen atmosphere at −78° C. was added 2.14 mL (30.1 mmol) DMSO dropwise over 5 min by syringe. After ten min., to this mixture was added a solution of 4.15 g (7.53 mmol) tert-butyl 4-{1-[3,5-bis(trifluoromethyl)phenyl]ethoxy}-2-(hydroxymethyl)-3-phenylpyrrolidine-1-carboxylate in 15 mL dry methylene chloride. The reaction mixture was stirred at −78° C. for 20 min, then 5.25 mL (37.7 mmol) TEA was added by syringe. The reaction mixture was stirred at −78° C. for 15 min then warmed to room temperature and stirred an additional hr. The reaction mixture was quenched with aq. 1N HCL (˜15 mL) and transferred to a separatory funnel. The reaction mixture was extracted with EtOAc (2×15 mL). The combined organic extracts were washed with water (15 mL) then brine (15 mL), dried over anhydrous sodium sulfate, filtered and evaporated under vacuum afford the title compound.

### Step B: tert-butyl (2S,3S,4R)-4-{(1R)-1-[3,5-bis(trifluoromethyl)phenyl]ethoxy}-3-(4-fluorophenyl)-2-[(1E)-3-methoxy-3-oxoprop-1-en-1-yl]pyrrolidine-1-carboxylate

To a solution of the crude tert-butyl (2R,3S,4R)-4-{(1R)-1-[3,5-bis(trifluoromethyl)phenyl]ethoxy}-3-(4-fluorophenyl)-2-formylpyrrolidine-1-carboxylate (step A, 4.49 mmol) in 40 mL dry methylene chloride under nitrogen atmosphere was added 1.80 g (5.39 mmol) (methoxycarbonylmethylene) triphenylphosphorane at 0° C. The resulting mixture was stirred at RT for 4 hr. The solvent was removed under vacuum and the residue was purified by Horizon MPLC using a gradient eluting system of 4-35% ethyl acetate in hexane to afford 2.43 g (90%) of the title compound.

### Step C: tert-butyl (2S,3S,4R)-4-{(1R)-1-[3,5-bis(trifluoromethyl)phenyl]ethoxy}-3-(4-fluorophenyl)-2-(3-methoxy-3-oxopropyl)pyrrolidine-1-carboxylate

To a solution of 2.42 g (4.0 mmol) tert-butyl (2S,3S,4R)-4-{(1R)-1-[3,5-bis(trifluoromethyl)phenyl]ethoxy}-3-(4-fluorophenyl)-2-[(1E)-3-methoxy-3-oxoprop-1-en-1-yl]pyrrolidine-1-carboxylate (step B) in 100 mL methanol under nitrogen atmosphere was added 400 mg 10% Pd—C catalyst. The resulting mixture was stirred under 36 psi of hydrogen at RT. After several hours, the catalyst was filtered through filter-aid and the solvent was removed under vacuum to afford 2.37 g (98%) of the title compound.

### Step D: 3-[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]propanoic acid

A solution of tert-butyl (2S,3S,4R)-4-{(1R)-1-[3,5-bis(trifluoromethyl)phenyl]ethoxy}-3-(4-fluorophenyl)-2-(3-methoxy-3-oxopropyl)pyrrolidine-1-carboxylate (6.69 g, 11.0 mmol) and lithium hydroxide monohydrate (2.31 g, 55.1 mmol) in 150 mL methanol was added 40 mL water. The mixture was heated at 30° C. for 16 hr. Upon removeal of volatiles, residue was dissolved in EtOAc. The pH of the solution was adjusted to 3 by addition of 2 N HCl. The aqueous was extracted by EtOAc (2×150 mL). Upon removal of EtOAc, the residue was dried to give the title compound.

### Step E: tert-butyl (2S,3S,4R)-4-{(1R)-1-[3,5-bis(trifluoromethyl)phenyl]ethoxy}-2-(4-diazo-3-oxobutyl)-3-(4-fluorophenyl)pyrrolidine-1-carboxylate

A solution of 3-[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]propanoic acid (4.10 g, 6.93 mmol) in 35 mL THF was added Et3N (11.8 mmol) and isobutyl chloroformate (10.4 mmol) at 0° C. After 1 h, the mixture was cooled to −78° C. and was added a solution of diazomethane (prepared from 15 g of Diazald in 100 mL ether). The mixture was allowed to warm to rt over 3.5 h. It was quenched with HOAc and was poured into ether. The organic phase was washed with NaHCO3 and NaCl, dried with Na2SO4. Upon removal of solvent, it was purified by Horizon MPLC using a gradient eluting system of 20-34% ethyl acetate in hexane to afford 2.57 g of the title compound.

### Step F: tert-butyl (2S,3S,4R)-4-{(1R)-1-[3,5-bis(trifluoromethyl)phenyl]ethoxy}-3-(4-fluorophenyl)-2-(4-methoxy-4-oxobutyl)pyrrolidine-1-carboxylate

To a solution of 2.57 g (4.14 mmol) tert-butyl (2S,3S,4R)-4-{(1R)-1-[3,5-bis(trifluoromethyl)phenyl]ethoxy}-2-(4-diazo-3-oxobutyl)-3-(4-fluorophenyl)pyrrolidine-1-carboxylate (intermediate step E) and 1.73 mL (12.4 mmol) Et3N in 30 mL methanol was added silver benzoate (47 mg, 0.21 mmol) and the solution was stirred at rt for 16 h. Upon removal of volatiles, the residue was purified by Horizon MPLC using a gradient eluting system of 16-34% ethyl acetate in hexane to afford 2.22 g of the title compound.

### Step G: 4-[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]butanoic acid

The title compound was prepared from tert-butyl (2S,3S,4R)-4-{(1R)-1-[3,5-bis(trifluoromethyl)phenyl]ethoxy}-3-(4-fluorophenyl)-2-(4-methoxy-4-oxobutyl)pyrrolidine-1-carboxylate (Step F) with the same method as in step D.

### Step G: (1S,2R,8aS)-2-{(1R)-1-[3,5-bis(trifluoromethyl)phenyl]ethoxy}-1-(4-fluorophenyl)hexahydroindolizin-5(1H)-one

A solution of 4-[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]butanoic acid (0.66 g, 1.09 mmol) in 30 mL of 4 N HCl in dioxane was stirred at rt for 10 hr. Upon removal of volatiles, the residue was dissolved in 30 mL CH2Cl2 and was added DMAP (26.9 mg, 0.22 mmol), DIEA (0.38 mL, 2.17 mmol) and EDC (0.42 g, 2.17 mmol). The mixture was stirred at rt for 16 hr. After removal of volatiles, the residue was purified by Horizon MPLC using a gradient eluting system of 50-100% ethyl acetate in hexane to afford 0.46 g (87%) of the title compound. MS: 490 (M+1).

### Example 2 and Example 3

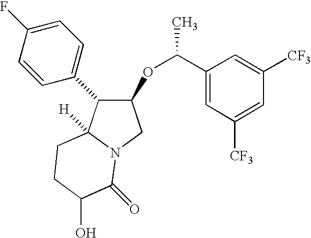

### (1S,2R,8aS)-2-{(1R)-1-[3,5-bis(trifluoromethyl)phenyl]ethoxy}-1-(4-fluorophenyl)-6 (R or S)-hydroxyhexahydroindolizin-5(1H)-one

To a solution of 1S,2R,8aS)-2-{(1R)-1-[3,5-bis(trifluoromethyl)phenyl]ethoxy}-1-(4-fluorophenyl)hexahydroindolizin-5(1H)-one (194 mg, 0.40 mmol) in 6 mL anhydrous THF under nitrogen atmosphere cooled to −78° C. via dry ice/acetone bath was added a solution of LHMDS (0.8 mL as 1 M solution in THF). The resulting mixture was stirred at −78° C. for ten minutes, and then allowed to warm to −20° C. over one hour. MoOPh (516 mg, 1.08 mmol) was then added as a solid to the solution and the resulting mixture was stirred in the dark by wrapping the round bottom flask with aluminum foil. After 10 min, the solution was warmed to room temperature for 100 min. The mixture was quenched with Na₂SO₃ solution and was partitioned with ethyl acetate and 2N HCl and the organic layer was then further washed with brine, dried over sodium sulfate, filtered through a fritted funnel, and concentrated in vacuum. The residue was purified by preparative TLC plate eluting with ethyl acetate/hexanes/2N NH3 in MeOH (10/10/1) to afford the two isomers as a mixture, which was separated by chiral OJ column on HPLC with EtOH/hexanes (1/9) to give the title compounds. Isomer 1 was named for the less polar isomer (35.9 mg), while isomer 2 was the more polar isomer (74.2 mg). MS: 506

### Example 4 and Example 5

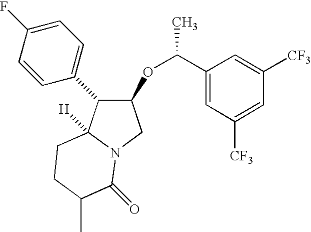

### (1S,2R,8aS)-2-{(1R)-1-[3,5-bis(trifluoromethyl)phenyl]ethoxy}-1-(4-fluorophenyl)-6 (R or S)-methylhexahydroindolizin-5(1H)-one

To a solution of 1S,2R,8aS)-2-{(1R)-1-[3,5-bis(trifluoromethyl)phenyl]ethoxy}-1-(4-fluorophenyl)hexahydroindolizin-5(1H)-one (99.6 mg, 0.20 mmol) in 3.5 mL anhydrous THF under nitrogen atmosphere cooled to −78° C. via dry ice/acetone bath was added a solution of LHMDS (0.33 mL as 1 M solution in THF). The resulting mixture was stirred at −78° C. for ten minutes, −40° C. for 20 minutes and then allowed to cooled to −78° C. To the solution was added iodomethane (0.051 mL, 0.81 mmol) and the mixture was stirred for 90 minutes. The mixture was quenched with NH4Cl solution (2 drops) and was warmed to rt. The solution was diluted with CH2Cl2 and was dried with Na₂SO₄, filtered through a fritted funnel, and concentrated in vacuo. The residue was purified by preparative TLC plate eluting with ethyl acetate/hexanes/2N NH₃ in MeOH (10/10/1) to afforded the title compounds as two isomers. Isomer I was named for the less polar isomer (41.2 mg), while isomer 2 was the more polar isomer (46.3 mg). MS: 504.

### Example 6 and Example 7

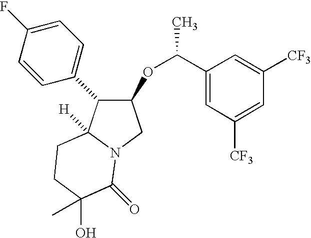

### (1S,2R,8aS)-2-{(1R)-1-[3,5-bis(trifluoromethyl)phenyl]ethoxy}-1-(4-fluorophenyl)-6 (R or S)-hydroxy-6-methylhexahydroindolizin-5(1H)-one

The title compounds were prepared from (1S,2R,8aS)-2-{(1R)-1-[3,5-bis(trifluoromethyl)phenyl]ethoxy}-1-(4-fluorophenyl)-6-methylhexahydroindolizin-5(1H)-one with the method described in Example 2. The two isomers were separated by chiral OD column on HPLC. Isomer 1 was named for the less polar isomer, while isomer 2 was the more polar isomer. MS: 520.

### Example 8

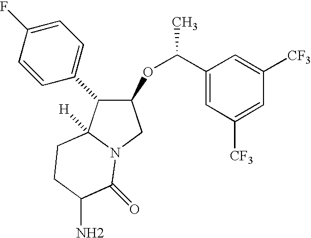

### (1S,2R,8aS)-6-amino-2-{(1R)-1-[3,5-bis(trifluoromethyl)phenyl]ethoxy}-1-(4-fluorophenyl)hexahydroindolizin-5(1H)-one

### Step A: methyl (1S,2R,8aS)-2-{(1R)-1-[3,5-bis(trifluoromethyl)phenyl]ethoxy}-1-(4-fluorophenyl)-5-oxooctahydroindolizine-6-carboxylate

To a solution of 1S,2R,8aS)-2-{(1R)-1-[3,5-bis(trifluoromethyl)phenyl]ethoxy}-1-(4-fluorophenyl)hexahydroindolizin-5(1H)-one (338 mg, 0.69 mmol) and LDA (0.69 mL, C=2M) in 3 mL THF under nitrogen atmosphere cooled to −78° C. via dry ice/acetone bath was added CO(OMe)₂ (0.070 mL, 0.83 mmol). The mixture was warmed to rt for 2 h and was quenched with NR₄Cl. The mixture was diluted with ether and was washed with brine, dried with Na₂SO₄, filtered through a fritted funnel, and concentrated in vacuo. The residue was purified by Horizon MPLC using a gradient eluting system of 50-100% ethyl acetate in hexane to afford 0.28 g (75%) of the title compound. MS: 548 (M+1).

### Step B: (1S,2R,8aS)-2-{(1R)-1-[3,5-bis(trifluoromethyl)phenyl]ethoxy}-1-(4-fluorophenyl)-5-oxooctahydroindolizine-6-carboxylic acid

The title compounds were prepared from methyl (1S,2R,8aS)-2-{(1R)-1-[3,5-bis(trifluoromethyl)phenyl]ethoxy}-1-(4-fluorophenyl)-5-oxooctahydroindolizine-6-carboxylate with the method described in Example 1, step D. MS: 534 (M+1).

### Step C: benzyl [(1S,2R,8aS)-2-{(1R)-1-[3,5-bis(trifluoromethyl)phenyl]ethoxy}-1-(4-fluorophenyl)-5-oxooctahydroindolizin-6-yl]carbamate

To a solution of (1S,2R,8aS)-2-{(1R)-1-[3,5-bis(trifluoromethyl)phenyl]ethoxy}-1-(4-fluorophenyl)-5-oxooctahydroindolizine-6-carboxylic acid (32 mg, 0.06 mmol) (intermediate step B) in 3 mL anhydrous toluene was added μL DPPA (0.052 mL, 0.24 mmol) and Et₃N (0.033 mL, 0.24 mmoL). The resulting solution was heated in 88° C. oil bath for 3.5 hr; then, cooled to 50° C. To the mixture was added 100 μL benzyl alcohol (0.062 mL) and the resulting solution was heated at 100° C. for 16 hr. Upon removal of volatiles, the residue was purified by reverse phase HPLC to give the title compound.

### Step D: (1S,2R,8aS)-6-amino-2-{(1R)-1-[3,5-bis(trifluoromethyl)phenyl]ethoxy}-1-(4-fluorophenyl)hexahydroindolizin-5(1H)-one

To a solution of benzyl [(1S,2R,8aS)-2-{(1R)-1-[3,5-bis(trifluoromethyl)phenyl]ethoxy}-1-(4-fluorophenyl)-5-oxooctahydroindolizin-6-yl]carbamate (4.3 mg) (Step C) in 5 mL ethanol was added 10 mg 10% palladium on carbon. The resulting suspension was shaken under 40 psi hydrogen atmosphere for 2 hours. The catalyst was filtered off and was dried to give the title compound. MS: 541.

### Example 9 and Example 10

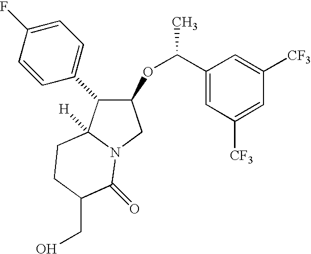

### (1S,2R,8aS)-2-{(1R)-1-[3,5-bis(trifluoromethyl)phenyl]ethoxy}-1-(4-fluorophenyl)-6 (R or S)-(hydroxymethyl)hexahydroindolizin-5(1H)-one

To a solution of methyl (1S,2R,8aS)-2-{(1R)-1-[3,5-bis(trifluoromethyl)phenyl]ethoxy}-1-(4-fluorophenyl)-5-oxooctahydroindolizine-6-carboxylate (20 mg, 0.037 mmol) (intermediate step A, example 8) in 3 mL anhydrous CH₂Cl₂ was added DIBAL-H (0.073 mL, 0.073 mmol) at −78° C. The mixture was slowly warmed to −20° C. over 1.5 hr and was quenched with HOAc. It was diluted with CH₂Cl₂ and was added Na₂SO₄.10 H₂O and stirred at rt for 0.5 h. The suspension was filtered through celite. Upon removal of volatiles, it was purified by reverse phase HPLC. The fast isomer was labeled as isomer D1. The slow isomer was labeled as isomer D2. MS: 520.

### Example 11 and Example 12

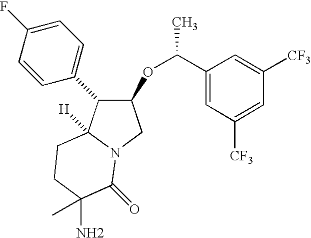

### (1S,2R,8aS)-6 (R or S)-amino-2-{(1R)-1-[3,5-bis(trifluoromethyl)phenyl]ethoxy}-1-(4-fluorophenyl)-6-methylhexahydroindolizin-5(1H)-one

### Step A: methyl (1S,2R,8aS)-2-{(1R)-1-[3,5-bis(trifluoromethyl)phenyl]ethoxy}-1-(4-fluorophenyl)-6-methyl-5-oxooctahydroindolizine-6-carboxylate

The title compound was prepared from methyl (1S,2R,8aS)-2-{(1R)-1-[3,5-bis(trifluoromethyl)phenyl]ethoxy}-1-(4-fluorophenyl)-5-oxooctahydroindolizine-6-carboxylate (intermediate, example 8, step A) with same procedure as example 4. MS: 562 (M+1).

### Step B: (1S,2R,8aS)-2-{(1R)-1-[3,5-bis(trifluoromethyl)phenyl]ethoxy}-1-(4-fluorophenyl)-6-methyl-5-oxooctahydroindolizine-6-carboxylic acid

A solution of methyl (1S,2R,8aS)-2-{(1R)-1-[3,5-bis(trifluoromethyl)phenyl]ethoxy}-1-(4-fluorophenyl)-6-methyl-5-oxooctahydroindolizine-6-carboxylate (30 mg, 0.053 mmol) and lithium hydroxide monohydrate (0.011 mg, 0.27 mmol) in 1 mL methanol was added 1 mL water and 1 mL THF. The mixture was heated at 60° C. for 1 hr. Upon removal of volatiles, residue was purified by reverse phase HPLC to give two isomers. The fast isomer was labeled as D1 and the slow isomer was labeled as D2. MS: 548 (M+1).

### Step C: (1S,2R,8aS)-6 (R or S)-amino-2-{(R)-1-[3,5-bis(trifluoromethyl)phenyl]ethoxy}-1-(4-fluorophenyl)-6-methylhexahydroindolizin-5(1H)-one

The title compounds were prepared from D1 and D2 isomers (step B) according to the procedures described in example 8 (steps C and D). MS: 555 (M+1).

### Example 13

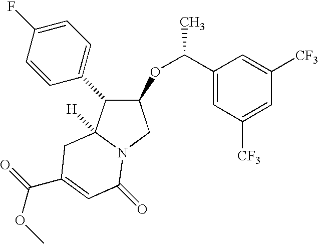

### Methyl (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizine-7-carboxylate

A solution of 4-tert-butyl 1-methyl 2-(dimethoxyphosphoryl)succinate (1.40 g, 4.73 mmol) in 15 mL of THF at 0° C. was added a LHMDS (5.09 mmol as a 1M solution in THF). After 1 hr, a solution of tert-butyl (2R,3S,4R)-4-{(1R)-1-[3,5-bis(trifluoromethyl)phenyl]ethoxy}-3-(4-fluorophenyl)-2-formylpyrrolidine-1-carboxylate (2.0 g, 3.63 mmol) in 15 mL of THF was added at 0° C. The resulting solution was allowed to warmed up to rt for 16 hr. The mixture was diluted with ether and was washed with brine. The organic phase was dried with Na₂SO₄, filtered and concentrated. The crude was purified by flash chromatography with EtOAc/hexanes (1:6 to 1:4). The product was dissolved in 20 mL of 4N HCl in dioxane and stirred at rt for 5 h. Volatiles were removed under vacuum. The crude was dissolved in 20 mL of CH₂Cl₂ and was added DMAP (78 mg, 0.64 mmol), N-(3-Dimethylaminopropyl)-N′-ethylcarbodiimide hydrochloride (1.22 g, 6.4 mmol) and diisopropyl ethylamine (1.39 mL, 8.0 mmol). After 12 h at rt, the mixture was diluted with ether and was washed with NaHCO₃ and brine. The organic phase was dried with Na₂SO₄, filtered and concentrated. The crude was purified by flash chromatography with EtOAc/hexanes (1:4 to 1:0) to afford the title compound. MS: 546 (M+1).

### Example 14

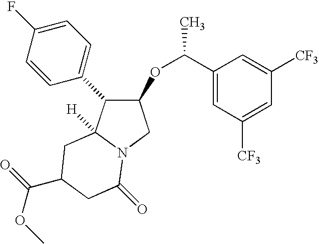

### Methyl (1S,2R,8aS)-2-{(1R)-1-[3,5-bis(trifluoromethyl)phenyl]ethoxy}-1-(4-fluorophenyl)-5-oxooctahydroindolizine-7-carboxylate

A solution of methyl (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizine-7-carboxylate (0.54 g, 0.99 mmol) in 30 mL of MeOH was added 150 mg of Pd/C (10%). The solution was shaken under 50 psi of H₂ for 7 h. The mixture was filtered through celite and was concentrated to afford the title compound. MS: 548 (M+1).

### Example 15

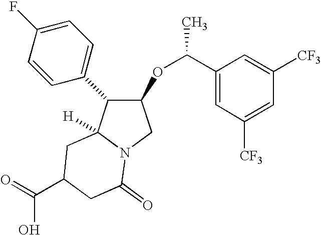

### (1S,2R,8aS)-2-{(1R)-1-[3,5-bis(trifluoromethyl)phenyl]ethoxy}-1-(4-fluorophenyl)-5-oxooctahydroindolizine-7-carboxylic acid

A solution of methyl (1S,2R,8aS)-2-{(1R)-1-[3,5-bis(trifluoromethyl)phenyl]ethoxy}-1-(4-fluorophenyl)-5-oxooctahydroindolizine-7-carboxylate (78 mg, 0.14 mmol) in 2 mL of MeOH was added a solution of LiOH.H₂O (23.9 mg, 0.57 mmol) in 0.8 mL of water. The resulting solution was heated at 40° C. for 2.5 hr. Upon removal of volatiles, the crude was purified by reverse phase HPLC to afford the title compound. MS: 534 (M+1).

### Example 16

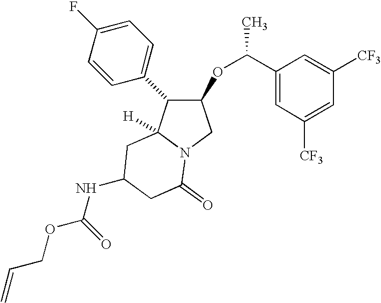

### Allyl [(1S,2R,8aS)-2-{(1R)-1-[3,5-bis(trifluoromethyl)phenyl]ethoxy}-1-(4-fluorophenyl)-5-oxooctahydroindolizin-7-yl]carbamate

A solution of (1S,2R,8aS)-2-{(1R)-1-[3,5-bis(trifluoromethyl)phenyl]ethoxy}-1-(4-fluorophenyl)-5-oxooctahydroindolizine-7-carboxylic acid (59.3 mg, 0.11 mmol), DPPA (0.048 mL, 0.22 mL) and Et₃N (0.031 mL, 0.22 mmol) in 3 mL of toluene was stirred at rt for 0.5 hr. To the mixture was aeed allyl alcohol (0.075 mL, 1.1 mmol) and was heated at 85° C. for 16 hr. Volatiles were removed and the crude was purified by preparative TLC with acetone/hexanes (1:3) to afford the title compound. MS: 589 (M+1).

### Example 17

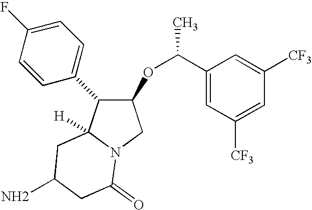

### (1S,2R,8aS)-7-amino-2-{(1R)-1-[3,5-bis(trifluoromethyl)phenyl]ethoxy}-1-(4-fluorophenyl)hexahydroindolizin-5(1H)-one

A solution of allyl [(1S,2R,8aS)-2-{(1R)-1-[3,5-bis(trifluoromethyl)phenyl]ethoxy}-1-(4-fluorophenyl)-5-oxooctahydroindolizin-7-yl]carbamate (30 mg, 0.051 mmol), Pd(PPh₃)₄ (12 mg, 0.1 mmol) and NaBH₄ (20 mg, 0.52 mmol) in 3 mL of THF was stirred at rt for 2 hr. To the mixture was added 5 mL of CH₃CN and 0.1 mL of 2 N HCl. After gas formation stopped, volatiles were removed and the crude was purified by reverse phase HPLC to afford the title compound. MS: 505 (M+1).

### Example 18

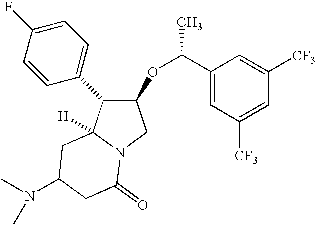

### 5 (1S,2R,8aS)-2-{(1R)-1-[3,5-bis(trifluoromethyl)phenyl]ethoxy}-7-(dimethylamino)-1-(4-fluorophenyl)hexahydroindolizin-5(1H)-one

A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(15 mg, 0.024 mmol), molecular sieves (4A, 30 mg), formaldehyde (0.022 mL, 0.288 mmol as 37% solution in water), Et₃N (0.027 mL, 0.19 mmol) and NaB(OAc)₃H (31 mg, 0.15 mmol) in 3 mL of THF was stirred at rt for 16 hr. The mixture was filtered through celite and the crude was purified by reverse phase HPLC to afford the title compound. MS: 533 (M+1).

### Example 19

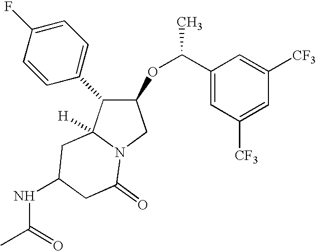

### N-[(1S,2R,8aS)-2-{(1R)-1-[3,5-bis(trifluoromethyl)phenyl]ethoxy}-1-(4-fluorophenyl)-5-oxooctahydroindolizin-7-yl]acetamide

A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one (15 mg, 0.024 mmol), pyridine (0.020 mL, 0.24 mmol) and acetic anhydride (0.012 mL, 0.12 mmol) in 3 mL of CH₂Cl₂ was stirred at rt for 16 hr. Upon removal of volatiles the crude was purified by reverse phase HPLC to afford the title compound. MS: 547 (M+1).

### Example 20

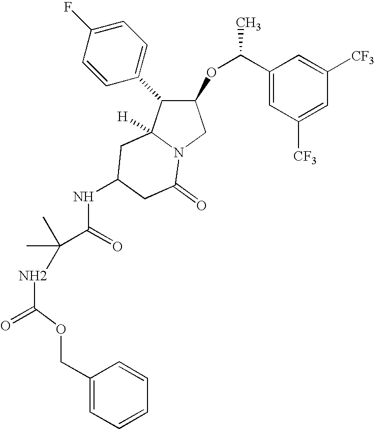

### Benzyl (2-{[(1S,2R,8aS)-2-{(1R)-1-[3,5-bis(trifluoromethyl)phenyl]ethoxy}-1-(4-fluorophenyl)-5-oxooctahydroindolizin-7-yl]amino}-1,1-dimethyl-2-oxoethyl)carbamate

A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(30 mg, 0.06 mmol), N-[(benzyloxy)carbonyl]-2-methylalanine (22 mg, 0.09 mmol), DCCI (19 mg, 0.09 mmol) and HOBt (13 mg, 0.09 mmol) in 10 mL of CH₂Cl₂ was stirred at rt for 16 hr. Upon removal of volatiles the crude was purified by Preparative TLC with MeOH/CH₂Cl₂ (5/95) to afford the title compound. MS: 724 (M+1).

### Example 21

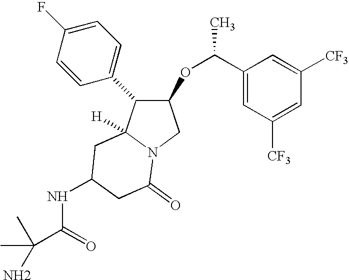

### N-[(1S,2R,8aS)-2-{(1R)-1-[3,5-bis(trifluoromethyl)phenyl]ethoxy}-1-(4-fluorophenyl)-5-oxooctahydroindolizin-7-yl]-2-methylalaninamide

The title compound was prepared from benzyl (2-{[(1S,2R,8aS)-2-{(1R)-1-[3,5-bis(trifluoromethyl)phenyl]ethoxy}-1-(4-fluorophenyl)-5-oxooctahydroindolizin-7-yl]amino}-1,1-dimethyl-2-oxoethyl)carbamate with the same procedure as Example 14. MS: 590 (M+1).

### Example 22

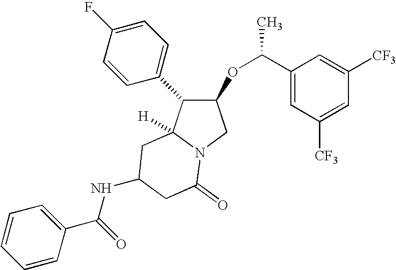

### N-[(1S,2R,8aS)-2-{(1R)-1-[3,5-bis(trifluoromethyl)phenyl]ethoxy}-1-(4-fluorophenyl)-5-oxooctahydroindolizin-7-yl]benzamide

The title compound was prepared from (1S,2R,8aS)-7-amino-2-{(1R)-1-[3,5-bis(trifluoromethyl)phenyl]ethoxy}-1-(4-fluorophenyl)hexahydroindolizin-5(1H)-one with the same procedure as Example 19. MS: 609 (M+1).

### Example 23

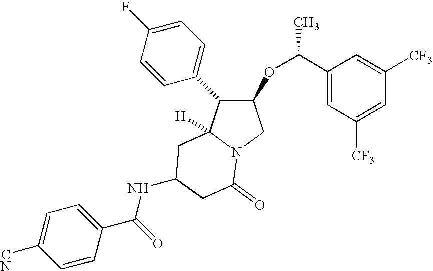

### N-[(1S,2R,8aS)-2-{(1R)-1-[3,5-bis(trifluoromethyl)phenyl]ethoxy}-1-(4-fluorophenyl)-5-oxooctahydroindolizin-7-yl]-4-cyanobenzamide

The title compound was prepared from (1S,2R,8aS)-7-amino-2-{(1R)-1-[3,5-bis(trifluoromethyl)phenyl]ethoxy}-1-(4-fluorophenyl)hexahydroindolizin-5(1H)-one with the same procedure as Example 19. MS: 634 (M+1).

### Example 24

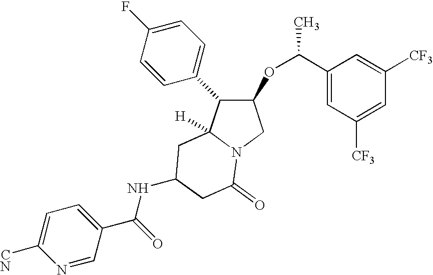

The title compound was prepared from (1S,2R,8aS)-7-amino-2-{(1R)-1-[3,5-bis(trifluoromethyl)phenyl]ethoxy}-1-(4-fluorophenyl)hexahydroindolizin-5(1H)-one with the same procedure as Example 20. MS: 635 (M+1).

### Example 25

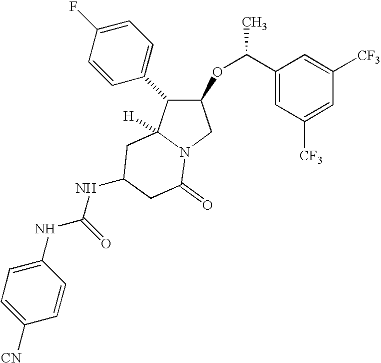

### N-[(1S,2R,8aS)-2-{(1R)-1-[3,5-bis(trifluoromethyl)phenyl]ethoxy}-1-(4-fluorophenyl)-5-oxooctahydroindolizin-7-yl]-N′-(4-isocyanophenyl)urea

### A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(10 mg, 0.02 mmol), 4-isocyanatobenzonitrile (7 mg, 0.04 mmol) and Et₃N (0.009 mL, 0.06 mmol) in 2 mL of CH₂Cl₂ was stirred at rt for 16 hr. Upon removal of volatiles the crude was purified by Preparative TLC with MeOH/CH₂Cl₂ (5/95) to afford the title compound. MS: 649 (M+1).

### Example 26

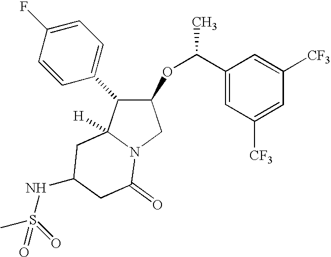

### N-[(1S,2R,8aS)-2-{(1R)-1-[3,5-bis(trifluoromethyl)phenyl]ethoxy}-1-(4-fluorophenyl)-5-oxooctahydroindolizin-7-yl]methanesulfonamide

A solution of (1S,2R,8aS)-7-amino-2-{(1R)-1-[3,5-bis(tri fluoromethyl)phenyl]ethoxy}-1-(4-fluorophenyl)hexahydroindolizin-5(1H)-one

(11.5 mg, 0.023 mmol), methanesulfonyl chloride (0.014 mL, 0.18 mmol) and Et₃N (0.032 mL, 0.23 mmol) in 2 mL of THF was stirred at rt for 2 hr. Upon removal of volatiles, the crude was purified by reverse phase HPLC to afford the title compound. MS: 583 (M+1).

### Example 27

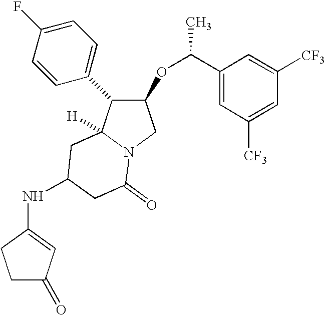

### (1S,2R,8aS)-2-{(1R)-1-[3,5-bis(trifluoromethyl)phenyl]ethoxy}-1-(4-fluorophenyl)-7-[(3-oxocyclopent-1-en-1-yl)amino]hexahydroindolizin-5(1H)-one

### A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(26 mg, 0.051 mmol), cyclopentane-1,3-dione (10 mg, 0.1 mmol) and TsOH.H₂O (2 mg, 0.01 mmol) in 3 mL of toluene was heated at 130° C. for 3 hr. Upon removal of volatiles, the crude was purified by reverse phase HPLC to afford the title compound. MS: 585 (M+1).

### Example 28

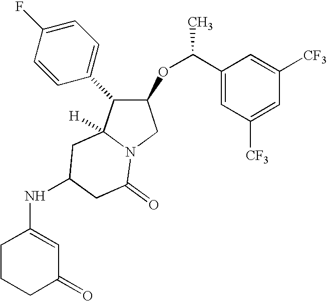

### (1S,2R,8aS)-2-{(1R)-1-[3,5-bis(trifluoromethyl)phenyl]ethoxy}-1-(4-fluorophenyl)-7-[(3-oxocyclohex-1-en-1-yl)amino]hexahydroindolizin-5(1H)-one

The title compound was prepared from (1S,2R,8aS)-7-amino-2-{(1R)-1-[3,5-bis(trifluoromethyl)phenyl]ethoxy}-1-(4-fluorophenyl)hexahydroindolizin-5(1H)-one with the same procedure as Example 27. MS: 599 (M+1).

### Example 29

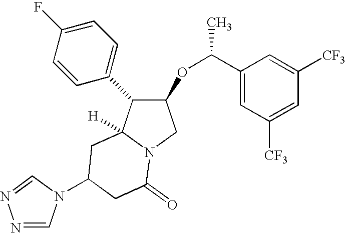

### (1S,2R,8aS)-2-{(1R)-1-[3,5-bis(trifluoromethyl)phenyl]ethoxy}-1-(4-fluorophenyl)-7-(4H-1,2,4-triazol-4-yl)hexahydroindolizin-5(1H)-one

A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(23 mg, 0.045 mmol), N′-[(1E)-(dimethylamino)methylene]-N,N-dimethylhydrazonoformamide (25.8 mg, 0.18 mmol) and TsOH.H2O (1.7 mg, 0.01 mmol) in 3.5 mL of toluene was heated at 115° C. for 20 hr. Upon removal of volatiles, the crude was purified by reverse phase HPLC to afford the title compound. MS: 557 (M+1).

### Example 30

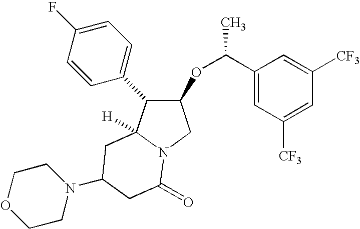

### (1S,2R,8aS)-2-{(1R)-1-[3,5-bis(trifluoromethyl)phenyl]ethoxy}-1-(4-fluorophenyl)-7-morpholin-4-ylhexahydroindolizin-5(1H)-one

### A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(23 mg, 0.045 mmol), Et3N (1 mL) and 1-bromo-2-(2-bromoethoxy)ethane (33 mg, 0.14 mmol) in 2 mL of DMF was heated at 100° C. for 20 hr. Upon removal of volatiles, the crude was purified by reverse phase HPLC to afford the title compound. MS: 575 (M+1).

### Example 31 and Example 32

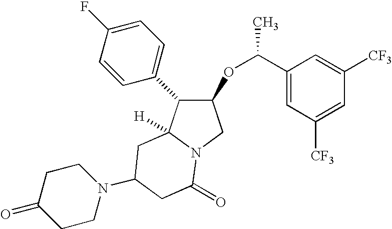

### (1S,2R,8aS)-2-{(1R)-1-[3,5-bis(trifluoromethyl)phenyl]ethoxy}-1-(4-fluorophenyl)-7-(4-oxopiperidin-1-yl)hexahydroindolizin-5(1H)-one

### A solution of (1S,2R,8aS)-7-amino-2-{(1R)-1-[3,5-bis(trifluoromethyl)phenyl]ethoxy}-1-(4-fluorophenyl)hexahydroindolizin-5(11)-one

(38 mg, 0.075 mmol) and K₂CO₃ (21 mg, 0.15 mmol) in 2 mL of ethanol was heated to reflux, then 1-ethyl-1-methyl-4-oxopiperidinium iodide (30 mg, 0.11 mmol) in 1.5 mL of water was added. Heating was continued for 1 hr and the mixture was poured into CH₂Cl₂. It was washed with water, dried over Na₂SO₄, filtered and concentrated. The crude was purified by Prep TLC with MeOH/CH₂Cl₂=5:95 to afford the title compounds (5.3 mg of fast isomer and 13.6 mg of slow isomer). MS: 587 (M+1).

### Example 33 and Example 34

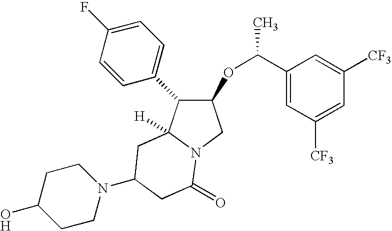

### (1S,2R,8aS)-2-{(1R)-1-[3,5-bis(trifluoromethyl)phenyl]ethoxy}-1-(4-fluorophenyl)-7-(4-hydroxypiperidin-1-yl)hexahydroindolizin-5(1H)-one

A solution of (1S,2R,8aS)-2-{(1R)-1-[3,5-bis(trifluoromethyl)phenyl]ethoxy}-1-(4-fluorophenyl)-7-(4-oxopiperidin-1-yl)hexahydroindolizin-5(1H)-one (8.0 mg, 0.014 mmol, slow isomer, Example 34) and NaBH4 (5.2 mg, 0.14 mmol) in 2 mL of MeOH was stirred at rt for 45 minutes and was quenched with 2 N HCl. The crude was worked up with CH₂Cl₂ and NaHCO₃ to afford the title compound as isomer A.

Isomer B was prepared from the fast isomer of (1S,2R,8aS)-2-{(1R)-1-[3,5-bis(trifluoromethyl)phenyl]ethoxy}-1-(4-fluorophenyl)-7-(4-oxopiperidin-1-yl)hexahydroindolizin-5(1H)-one with the same procedure. MS: 589 (M+1).

### Example 35 and Example 36

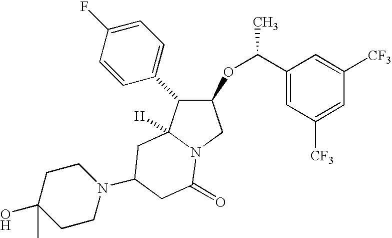

### (1S,2R,8aS)-2-{(1R)-1-[3,5-bis(trifluoromethyl)phenyl]ethoxy}-1-(4-fluorophenyl)-7-(4-hydroxy-4-methylpiperidin-1-yl)hexahydroindolizin-5(1H)-one

A solution of (1S,2R,8aS)-2-{(1R)-1-[3,5-bis(trifluoromethyl)phenyl]ethoxy}-1-(4-fluorophenyl)-7-(4-oxopiperidin-1-yl)hexahydroindolizin-5(1H)-one (28.1 mg, 0.048 mmol, slow isomer, Example 34) in 3 mL of THF was added MeMgBr (0.068 mL, 0.096 mmol) at 0° C. After 1 hr, it was diluted with CH₂Cl₂ and was quenched with Na₂SO₄.10H₂O and was filtered through celite. The crude was purified by prep TLC with MeOH/CH₂CL₂ to afford the title compound as isomer A.

Isomer B was prepared from the fast isomer of (1S,2R,8aS)-2-{(1R)-1-[3,5-bis(trifluoromethyl)phenyl]ethoxy}-1-(4-fluorophenyl)-7-(4-oxopiperidin-1-yl)hexahydroindolizin-5(1H)-one with the same procedure. MS: 603 (M+1).

### Example 37 (Isomer A) and Example 38 (Isomer B)

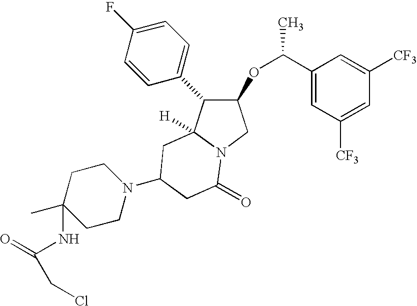

### N-{1-[(1S,2R,8aS)-2-{(1R)-1-[3,5-bis(trifluoromethyl)phenyl]ethoxy}-1-(4-fluorophenyl)-5-oxooctahydroindolizin-7-yl]-4-methylpiperidin-4-yl}-2-chloroacetamide

### A solution of (1S,2R,8aS)-2-{(1R)-1-[3,5-bis(trifluoromethyl)phenyl]ethoxy}-1-(4-fluorophenyl)-7-(4-hydroxy-4-methylpiperidin-1-yl)hexahydroindolizin-5(1H)-one

(28.1 mg, 0.048 mmol, isomer A, Example 37) in 2 mL of chloroacetonitrile was added 0.2 mL of H₂SO₄ (98%) at 0° C. After 1 hr, it was warmed to rt for 2 h. The reaction was quenched with K₂CO₃ and water and was extracted with CH₂Cl₂. Organic phase was dried with Na₂SO₄, filtered and concentrated. The crude was purified by prep TLC with MeOH/CH₂Cl₂ to afford the title compound as isomer A.

Isomer B was prepared from the isomer B of (1S,2R,8aS)-2-{(1R)-1-[3,5-bis(trifluoromethyl)phenyl]ethoxy}-1-(4-fluorophenyl)-7-(4-hydroxy-4-methylpiperidin-1-yl)hexahydroindolizin-5(1H)-one with the same procedure. MS: 679 (M+1).

### Example 39

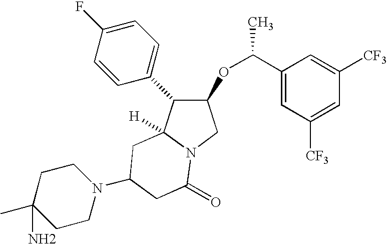

### (1S,2R,8aS)-7-(4-amino-4-methylpiperidin-1-yl)-2-{(1R)-1-[3,5-bis(trifluoromethyl)phenyl]ethoxy}-1-(4-fluorophenyl)hexahydroindolizin-5(1H)-one

A solution of N-{1-[(1S,2R,8aS)-2-{(1R)-1-[3,5-bis(trifluoromethyl)phenyl]ethoxy}-1-(4-fluorophenyl)-5-oxooctahydroindolizin-7-yl]-4-methylpiperidin-4-yl}-2-chloroacetamide (6 mg, 0.0089 mmol, isomer A, Example 38) and thiourea (6.7 mg, 0.088 mmol) in 2 mL of ethanol was added 0.4 mL of HOAc and was heated at 106° C. for 3 hr. Upon removal of volatiles, the crude was purified by reverse phase HPLC to afford the title compound. MS: 602 (M+1).

### Example 40

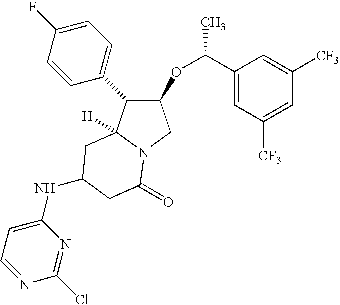

### (1S,2R,8aS)-2-{(1R)-1-[3,5-bis(trifluoromethyl)phenyl]ethoxy}-7-[(2-chloropyrimidin-4-yl)amino]-1-(4-fluorophenyl)hexahydroindolizin-5(1H)-one

### A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(32 mg, 0.064 mmol), 2,4-dichloropyrimidine (15 mg, 0.1 mmol) and Et₃N (0.028 mL, 0.19 mmol) in 2 mL of MeOH was heated at 110° C. in a sealed tube for 16 hr. Upon removal of volatiles, the crude was purified by reverse phase HPLC to afford the title compound. MS: 617 (M+1).

### Example 41

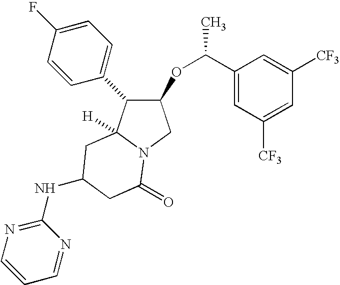

### (1S,2R,8aS)-2-{(1R)-1-[3,5-bis(trifluoromethyl)phenyl]ethoxy}-1-(4-fluorophenyl)-7-(pyrimidin-2-ylamino)hexahydroindolizin-5(1 h)-one

### A solution of (1S,2R,8aS)-7-amino-2-{(1R)-1-[3,5-bis(trifluoromethyl)phenyl]ethoxy}-1-(4-fluorophenyl)hexahydroindolizin-5(1H)-one

(15 mg, 0.03 mmol), 2-bromopyrimidine (10 mg, 0.06 mmol) and K₂CO₃ (21 mg, 0.15 mmol) in 2 mL of DMF was heated at 110° C. for 16 hr. Upon removal of volatiles, the crude was purified by reverse phase HPLC to afford the title compound. MS: 583 (M+1).

### (1S,2R,8aS)-2-{(1R)-1-[3,5-bis(trifluoromethyl)phenyl]ethoxy}-1-(4-fluorophenyl)-7-(pyrimidin-2-ylamino)hexahydroindolizin-5(1H)-one

### Example 42

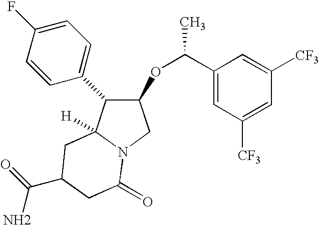

### (1S,2R,8aS)-2-{(1R)-1-[3,5-bis(trifluoromethyl)phenyl]ethoxy}-1-(4-fluorophenyl)-5-oxooctahydroindolizine-7-carboxamide

### A solution of (1S,2R,8aS)-2-{(1R)-1-[3,5-bis(trifluoromethyl)phenyl]ethoxy}-1-(4-fluorophenyl)-5-oxooctahydroindolizine-7-carboxylic acid

(100 mg, 0.19 mmol) in 20 mL of THF was added Et₃N (0.028 mL, 0.2 mmol) and ethyl chloroformate (0.02 mL, 0.2 mmol) at −10° C. and was stirred for 1 h. Ammonia gas was passed through the solution for 1 h and the mixture was allowed to warm to rt for 16 hr. Upon removal of volatiles, the crude was purified by prep TLC to with MeOH/CH₂Cl₂=10:90 to afford the title compound. MS: 533 (M+1).

### Example 43

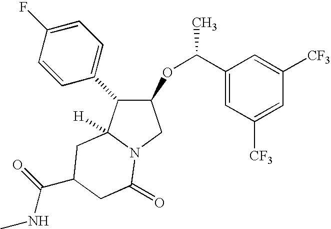

### (1S,2R,8aS)-2-{(1R)-1-[3,5-bis(trifluoromethyl)phenyl]ethoxy}-1-(4-fluorophenyl)-N-methyl-5-oxooctahydroindolizine-7-carboxamide

The title compound was prepared from (1S,2R,8aS)-2-{(R)-1-[3,5-bis(trifluoromethyl)phenyl]ethoxy}-1-(4-fluorophenyl)-5-oxooctahydroindolizine-7-carboxylic acid with the same procedure as Example 20. MS: 547 (M+1).

### Example 44

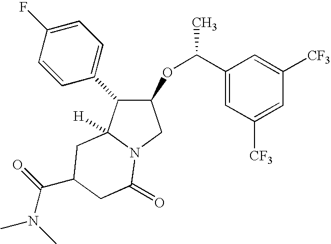

### (1S,2R,8aS)-2-{(1R)-1-[3,5-bis(trifluoromethyl)phenyl]ethoxy}-1-(4-fluorophenyl)-N,N-dimethyl-5-oxooctahydroindolizine-7-carboxamide

The title compound was prepared from (1S,2R,8aS)-2-{(1R)-1-[3,5-bis(trifluoromethyl)phenyl]ethoxy}-1-(4-fluorophenyl)-5-oxooctahydroindolizine-7-carboxylic acid with the same procedure as Example 20. MS: 561 (M+1).

### Example 45 and Example 46

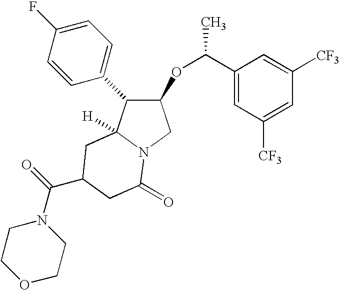

### (1S,2R,8aS)-2-{(1R)-1-[3,5-bis(trifluoromethyl)phenyl]ethoxy}-1-(4-fluorophenyl)-7-(morpholin-4-ylcarbonyl)hexahydroindolizin-5(1H)-one

The title compounds was prepared from (1S,2R,8aS)-2-{(1R)-1-[3,5-bis(trifluoromethyl)phenyl]ethoxy}-1-(4-fluorophenyl)-5-oxooctahydroindolizine-7-carboxylic acid with the same procedure as Example 20. The fast isomer on TLC plate was label as isomer A and the slow isomer as isomer B. MS: 603 (M+1).

### Example 47

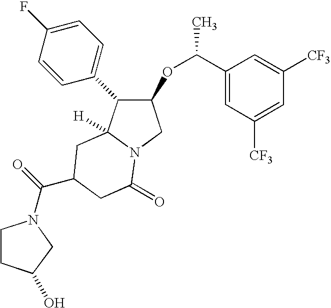

### (1s,2R,8aS)-2-{(1R)-1-[3,5-bis(trifluoromethyl)phenyl]ethoxy}-1-(4-fluorophenyl)-7-{[(3R)-3-hydroxypyrrolidin-1-yl]carbonyl}hexahydroindolizin-5(1H)-one

MS: 603 (M+1).

### Example 48

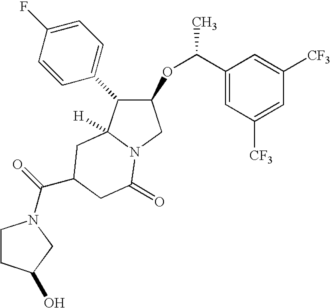

### (1S,2R,8aS)-2-{(1R)-1-[3,5-bis(trifluoromethyl)phenyl]ethoxy}-1-(4-fluorophenyl)-7-{[(3S)-3-hydroxypyrrolidin-1-yl]carbonyl}hexahydroindolizin-5(1H)-one

MS: 603 (M+1).

### Example 49

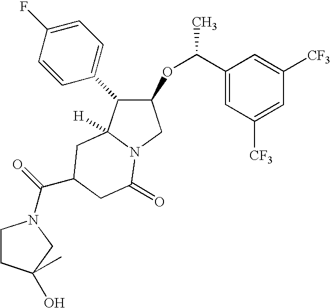

### (1S,2R,8aS)-2-{(1R)-1-[3,5-bis(trifluoromethyl)phenyl]ethoxy}-1-(4-fluorophenyl)-7-[(3-hydroxy-3-methylpyrrolidin-1-yl)carbonyl]hexahydroindolizin-5(1H)-one

MS: 617 (M+1).

### Example 50

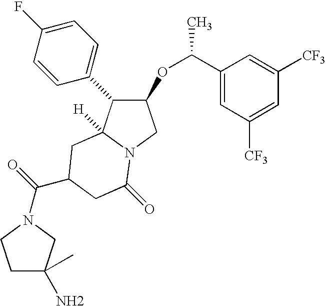

### (1S,2R,8aS)-7-[(3-amino-3-methylpyrrolidin-1-yl)carbonyl]-2-{(1R)-1-[3,5-bis(trifluoromethyl)phenyl]ethoxy}-1-(4-fluorophenyl)hexahydroindolizin-5(1H)-one

MS: 616 (M+1).

### Example 51

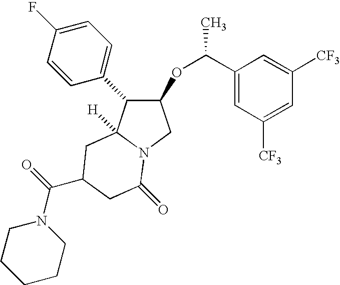

### (1S,2R,8aS)-2-{(1R)-1-[3,5-bis(trifluoromethyl)phenyl]ethoxy}-1-(4-fluorophenyl)-7-(piperidin-1-ylcarbonyl)hexahydroindolizin-5(1H)-one

MS: 601 (M+1).

### Example 52

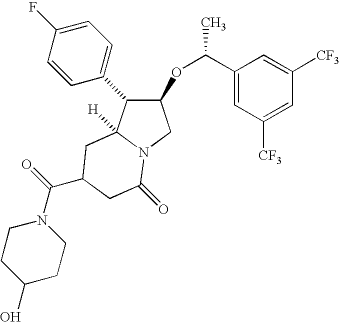

### (1S,2R,8aS)-2-{(1R)-1-[3,5-bis(trifluoromethyl)phenyl]ethoxy}-1-(4-fluorophenyl)-7-[(4-hydroxypiperidin-1-yl)carbonyl]hexahydroindolizin-5(1H)-one

MS: 617 (M+1).

### Example 53 and Example 54

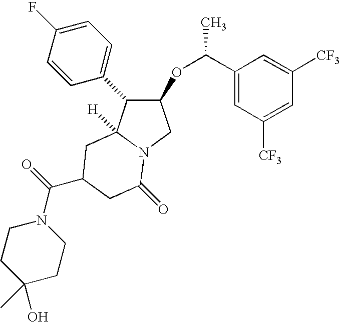

### (1S,2R,8aS)-2-{(1R)-1-[3,5-bis(trifluoromethyl)phenyl]ethoxy}-1-(4-fluorophenyl)-7-[(4-hydroxy-4-methylpiperidin-1-yl)carbonyl]hexahydroindolizin-5(1H)-one

MS: 631 (M+1).

### Example 55

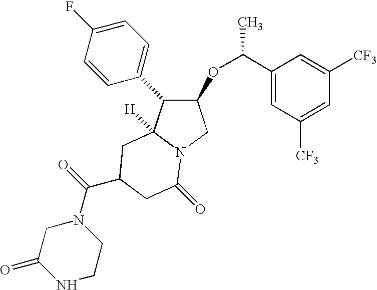

### (1S,2R,8aS)-2-{(1R)-1-[3,5-bis(trifluoromethyl)phenyl]ethoxy}-1-(4-fluorophenyl)-7-[(3-oxopiperazin-1-yl)carbonyl]hexahydroindolizin-5(1H)-one

MS: 616 (M+1).

### Example 56

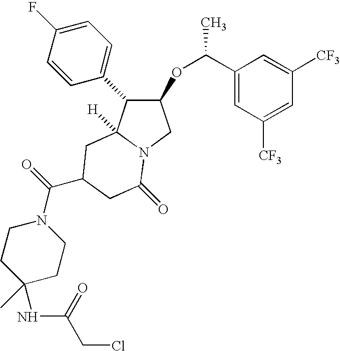

### N-(1-{[(1S,2R,8aS)-2-{(R)-1-[3,5-bis(trifluoromethyl)phenyl]ethoxy}-1-(4-fluorophenyl)-5-oxooctahydroindolizin-7-yl]carbonyl}-4-methylpiperidin-4-yl)-2-chloroacetamide

MS: 707 (M+1).

### Example 57

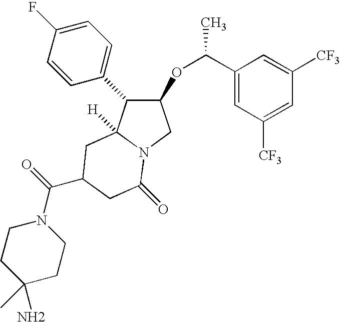

### (1S,2R,8aS)-7-[(4-amino-4-methylpiperidin-1-yl)carbonyl]-2-{(1R)-1-[3,5-bis(trifluoromethyl)phenyl]ethoxy}-1-(4-fluorophenyl)hexahydroindolizin-5(1H)-one

MS: 630 (M+1).

### Example 58 and Example 59

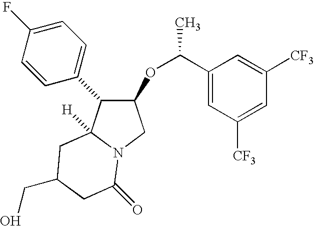

### (1S,2R,8aS)-2-{(1R)-1-[3,5-bis(trifluoromethyl)phenyl]ethoxy}-1-(4-fluorophenyl)-7-(hydroxymethyl)hexahydroindolizin-5(1H)-one

A solution of methyl (1S,2R,8aS)-2-{(1R)-1-[3,5-bis(trifluoromethyl)phenyl]ethoxy}-1-(4-fluorophenyl)-5-oxooctahydroindolizine-7-carboxylate (0.076 g, 0.14 mmol) in 3 mL of THF was added LAH (0.27 mL, 0.27 mmol) at −78° C. After 30 minutes, it was quenched with HOAc. The mixture was poured into EtOAc and was washed with NH4Cl and NaHCO₃, dried with Na₂SO₄, filtered and concentrated to provide its aldehyde form. The aldehyde was dissolved in 3 mL of MeOH and was added NaBH₄ (42.5 mg, 1.12 mmol) at 0° C. After 0.5 hr, it was quenched with 2N HCl. The mixture was poured into CH₂Cl₂ and was washed with brine and NaHCO₃, dried with Na₂SO₄, filtered and concentrated. The crude was purified by HPLC with chiral OD column to provide the title compounds. MS: 520 (M+1).

### Example 60

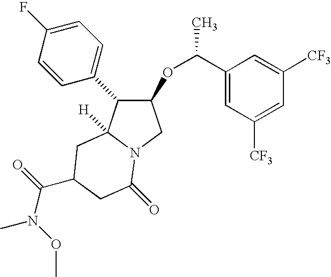

### (1S,2R,8aS)-2-{(1R)-1-[3,5-bis(trifluoromethyl)phenyl]ethoxy}-1-(4-fluorophenyl)-N-methoxy-N-methyl-5-oxooctahydroindolizine-7-carboxamide

MS: 577 (M+1).

### Example 61

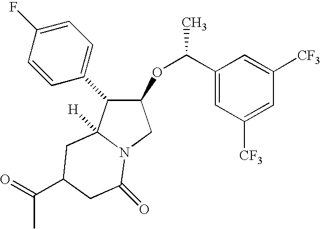

### (1S,2R,8aS)-7-acetyl-2-{(1R)-1-[3,5-bis(trifluoromethyl)phenyl]ethoxy}-1-(4-fluorophenyl)hexahydroindolizin-5(1H)-one

### A solution of (1S,2R,8aS)-2-{(1R)-1-[3,5-bis(trifluoromethyl)phenyl]ethoxy}-1-(4-fluorophenyl)-N-methoxy-N-methyl-5-oxooctahydroindolizine-7-carboxamide

(0.102 g, 0.18 mmol) in 5 mL of THF was added MeMgBr (0.50 mL, 0.72 mmol) at −78° C. After 30 minutes, it was warmed to rt for 1 hr and was quenched with Na₂SO₄. 10H₂O. The mixture was diluted with EtOAc, filtered and concentrated. The crude was purified by prep TLC with MeOH/CH₂Cl₂ to provide the title compound. MS: 532 (M+1).

### Example 62 and Example 63

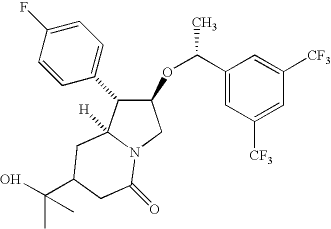

### (1S,2R,8aS)-2-{(1R)-1-[3,5-bis(trifluoromethyl)phenyl]ethoxy}-1-(4-fluorophenyl)-7-(1-hydroxy-1-methylethyl)hexahydroindolizin-5(1H)-one

The title compound was prepared from (1S,2R,8aS)-7-acetyl-2-{(1R)-1-[3,5-bis(trifluoromethyl)phenyl]ethoxy}-1-(4-fluorophenyl)hexahydroindolizin-5(1H)-one with the same procedure as Example 35. MS: 548 (M+1).

### Example 64 and 65

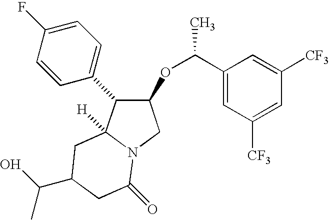

### (1S,2R,8aS)-2-{(1R)-1-[3,5-bis(trifluoromethyl)phenyl]ethoxy}-1-(4-fluorophenyl)-7-(1-hydroxyethyl)hexahydroindolizin-5(1H)-one

The title compound was prepared from (1S,2R,8aS)-7-acetyl-2-{(1R)-1-[3,5-bis(trifluoromethyl)phenyl]ethoxy}-1-(4-fluorophenyl)hexahydroindolizin-5(1H)-one with the same procedure as Example 33. MS: 534 (M+1).

### Example 66

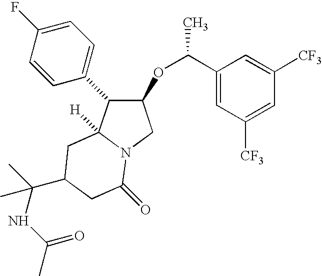

### N-{1-[(1S,2R,8aS)-2-{(1R)-1-[3,5-bis(trifluoromethyl)phenyl]ethoxy}-1-(4-fluorophenyl)-5-oxooctahydroindolizin-7-yl]-1-methylethyl}acetamide

The title compound was prepared from (1S,2R,8aS)-2-{(1R)-1-[3,5-bis(trifluoromethyl)phenyl]ethoxy}-1-(4-fluorophenyl)-7-(1-hydroxy-1-methylethyl)hexahydroindolizin-5(1H)-one and acetonitrile with the same procedure as Example 37. MS: 589 (M+1).

### Example 67

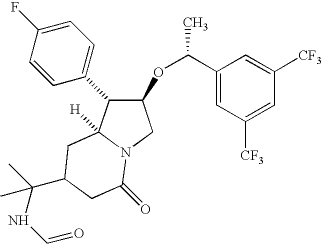

### N-{1-[(1S,2R,8aS)-2-{(1R)-1-[3,5-bis(trifluoromethyl)phenyl]ethoxy}-1-(4-fluorophenyl)-5-oxooctahydroindolizin-7-yl]-1-methylethyl}formamide

The title compound was prepared from (1S,2R,8aS)-2-{(1R)-1-[3,5-bis(trifluoromethyl)phenyl]ethoxy}-1-(4-fluorophenyl)-7-(1-hydroxy-1-methylethyl)hexahydroindolizin-5(1H)-one and NaCN with the same procedure as Example 37. MS: 575 (M+1).

### Example 68 and Example 69

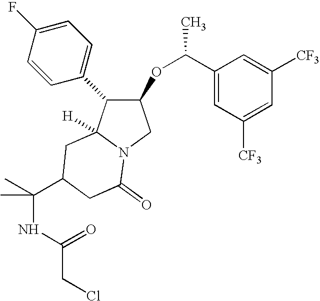

### N-{1-[(1S,2R,8aS)-2-{(1R)-1-[3,5-bis(trifluoromethyl)phenyl]ethoxy}-1-(4-fluorophenyl)-5-oxooctahydroindolizin-7-yl]-1-methylethyl}-2-chloroacetamide

The title compound was prepared from (1S,2R,8aS)-2-{(1R)-1-[3,5-bis(trifluoromethyl)phenyl]ethoxy}-1-(4-fluorophenyl)-7-(1-hydroxy-1-methylethyl)hexahydroindolizin-5(1H)-one and chloroacetonitrile with the same procedure as

### Example 37

MS: 623 (M+1).

### Example 70 and Example 71

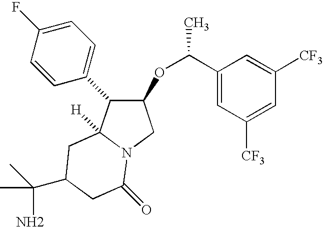

(1S,2R,8aS)-7-(1-amino-1-methylethyl)-2-{(1R)-1-[3,5-bis(trifluoromethyl)phenyl]ethoxy}-1-(4-fluorophenyl)hexahydroindolizin-5(1H)-one

The title compound was prepared from N-{1-[(1S,2R,8aS)-2-{(1R)-1-[3,5-bis(trifluoromethyl)phenyl]ethoxy}-1-(4-fluorophenyl)-5-oxooctahydroindolizin-7-yl]-1-methylethyl}-2-chloroacetamide with the same procedure as Example 39. MS: 547 (M+1).

### Example 72 and Example 73

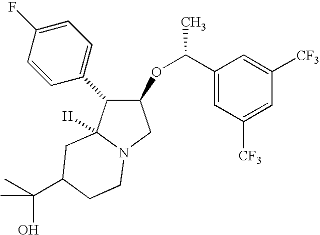

### 2-[(1S,2R,8aS)-2-{(1R)-1-[3,5-bis(trifluoromethyl)phenyl]ethoxy}-1-(4-fluorophenyl)octahydroindolizin-7-yl]propan-2-ol

A solution of (1S,2R,8aS)-2-{(1R)-1-[3,5-bis(trifluoromethyl)phenyl]ethoxy}-1-(4-fluorophenyl)-7-(1-hydroxy-1-methylethyl)hexahydroindolizin-5(1H)-one (18 mg, 0.036 mmol) in 3 mL of THF was added BH₃.5Me₂ (0.18 mL, 0.36 mmol) and the solution was heated at reflux for 2 h. Upon removeal of volatiles, the crude was dissolved in 2 mL of ethanol and was heated at 96° C. for 2.5 hr. Upon removal of volatiles, the residue was purified by prep TLC with MeOH/CH₂Cl₂ to afford the title compounds. MS: 534 (M+1).

### Example 74

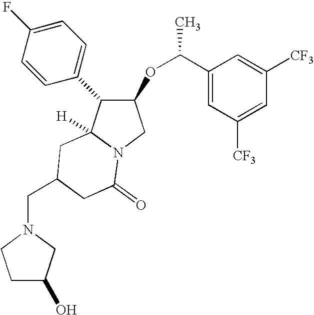

### (1S,2R,8aS)-2-{(1R)-1-[3,5-bis(trifluoromethyl)phenyl]ethoxy}-1-(4-fluorophenyl)-7-{[(3S)-3-hydroxypyrrolidin-1-yl]methyl}hexahydroindolizin-5(1H)-one

A solution of (1S,2R,8aS)-2-{(1R)-1-[3,5-bis(trifluoromethyl)phenyl]ethoxy}-1-(4-fluorophenyl)-7-(hydroxymethyl)hexahydroindolizin-5(1H)-one

(22 mg, 0.04 mmol) in 5 mL of CH₂Cl₂ was added Et₃N (0.014 mL, 0.06 mmol) and methanesulfonyl chloride (0.006 mL, 0.048 mmol) at rt. After 16 hr, volatiles were removed and was redissolved in 2 mL of DMF and (3S)-pyrrolidin-3-ol (0.011 mL, 0.08 mmol) was added. The solution was heated at 80° C. for 12 hr. Upon removal of volatiles, the residue was pprified by prep TLC with NH3-MeOH(2M)/CH₂Cl₂=4:96 to afford the title compounds. MS: 589 (M+1).

### Example 75

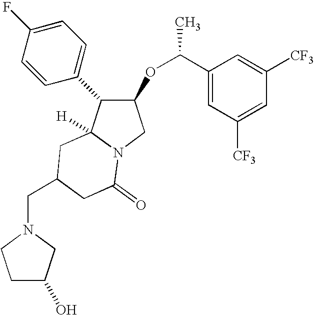

### (1S,2R,8aS)-2-{(1R)-1-[3,5-bis(trifluoromethyl)phenyl]ethoxy}-1-(4-fluorophenyl)-7-{[(3R)-3-hydroxypyrrolidin-1-yl]methyl}hexahydroindolizin-5(1H)-one

MS: 589 (M+1).

### Example 76

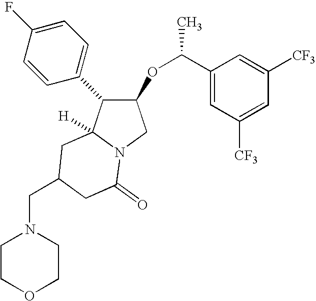

### (1S,2R,8aS)-2-{(1R)-1-[3,5-bis(trifluoromethyl)phenyl]ethoxy}-1-(4-fluorophenyl)-7-(morpholin-4-ylmethyl)hexahydroindolizin-5(1H)-one

MS: 589 (M+1).

### Example 77 and Example 78

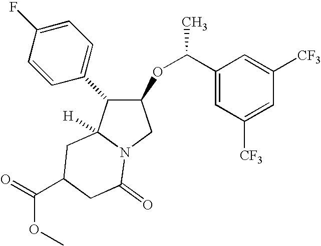

### Methyl (1S,2R,8aS)-2-{(1R)-1-[3,5-bis(trifluoromethyl)phenyl]ethoxy}-1-(4-fluorophenyl)octahydroindolizine-7-carboxylate

The title compound was prepared from methyl (1S,2R,8aS)-2-{(1R)-1-[3,5-bis(trifluoromethyl)phenyl]ethoxy}-1-(4-fluorophenyl)-5-oxooctahydroindolizine-7-carboxylate with the same procedure as Example 73. MS: 534 (M+1).

### Example 79 and Example 80

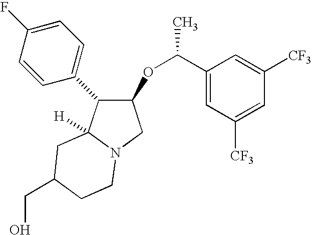

### [(1S,2R,8aS)-2-{(1R)-1-[3,5-bis(trifluoromethyl)phenyl]ethoxy}-1-(4-fluorophenyl)octahydroindolizin-7-yl]methanol

The title compounds were isolated as side products from Example 79 and 80. MS: 506 (M+1).

### Example 81

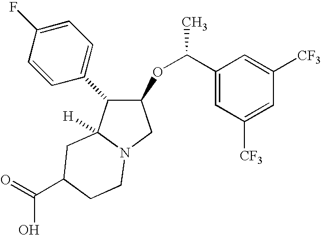

### (1S,2R,8aS)-2-{(1R)-1-[3,5-bis(trifluoromethyl)phenyl]ethoxy}-1-(4-fluorophenyl)octahydroindolizine-7-carboxylic acid

The title compound was prepared from methyl (1S,2R,8aS)-2-{(1R)-1-[3,5-bis(trifluoromethyl)phenyl]ethoxy}-1-(4-fluorophenyl)octahydroindolizine-7-carboxylate with the same procedure as Example 15. MS: 520 (M+1).

### Example 82

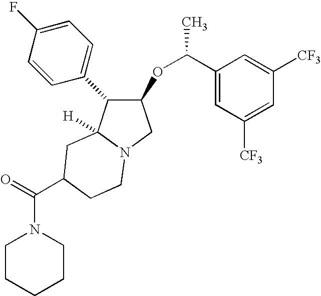

### (1S,2R,8aS)-2-{(1R)-1-[3,5-bis(trifluoromethyl)phenyl]ethoxy}-1-(4-fluorophenyl)-7-(piperidin-1-ylcarbonyl)octahydroindolizine

The title compound was prepared from (1S,2R,8aS)-2-{(1R)-1-[3,5-bis(trifluoromethyl)phenyl]ethoxy}-1-(4-fluorophenyl)octahydroindolizine-7-carboxylic acid with the same procedure as Example 20. MS: 587 (M+1).

### Example 83

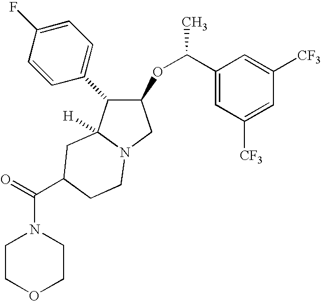

### (1S,2R,8aS)-2-{(1R)-1-[3,5-bis(trifluoromethyl)phenyl]ethoxy}-1-(4-fluorophenyl)-7-(morpholin-4-ylcarbonyl)octahydroindolizine

The title compound was prepared from (1S,2R,8aS)-2-{(1R)-1-[3,5-bis(trifluoromethyl)phenyl]ethoxy}-1-(4-fluorophenyl)octahydroindolizine-7-carboxylic acid with the same procedure as Example 20. MS: 589 (M+1).

### Example 84

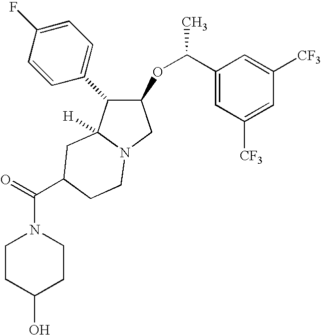

### 1-{[(1S,1R,8aS)-2-{(1R)-1-[3,5-bis(trifluoromethyl)phenyl]ethoxy}-1-(4-fluorophenyl)octahydroindolizin-7-yl]carbonyl}piperidin-4-ol

The title compound was prepared from (1S,2R,8aS)-2-{(1R)-1-[3,5-bis(trifluoromethyl)phenyl]ethoxy}-1-(4-fluorophenyl)octahydroindolizine-7-carboxylic acid with the same procedure as Example 20. MS: 603 (M+1).

### Example 85 and Example 86

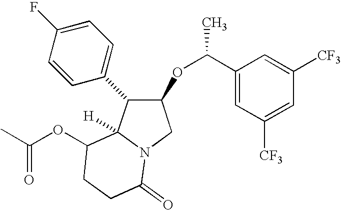

### (1S,2R,8aR)-2-{(1R)-1-[3,5-bis(trifluoromethyl)phenyl]ethoxy}-1-(4-fluorophenyl)-5-oxooctahydroindolizin-8-yl acetate

### Step A: tert-butyl (2R,3S,4R)-4-{(1R)-1-[3,5-bis(trifluoromethyl)phenyl]ethoxy}-3-(4-fluorophenyl)-2-(1-hydroxypent-4-en-1-yl)pyrrolidine-1-carboxylate

The title compound was prepared from tert-butyl (2R,3S,4R)-4-{(1R)-1-[3,5-bis(trifluoromethyl)phenyl]ethoxy}-3-(4-fluorophenyl)-2-formylpyrrolidine-1-carboxylate with the same procedure as Example 35.

### Step B: tert-butyl (2R,3S,4R)-2-[1-(acetyloxy)pent-4-en-1-yl]-4-{(1R)-1-[3,5-bis(trifluoromethyl)phenyl]ethoxy}-3-(4-fluorophenyl)pyrrolidine-1-carboxylate

The title compound was prepared from tert-butyl (2R,3S,4R)-4-{(1R)-1-[3,5-bis(trifluoromethyl)phenyl]ethoxy}-3-(4-fluorophenyl)-2-(1-hydroxypent-4-en-1-yl)pyrrolidine-1-carboxylate with the same procedure as Example 19.

### Step C: 4-(acetyloxy)-4-[(2R,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]butanoic acid

A solution of tert-butyl (2R,3S,4R)-2-[1-(acetyloxy)pent-4-en-1-yl]-4-{(1R)-1-[3,5-bis(trifluoromethyl)phenyl]ethoxy}-3-(4-fluorophenyl)pyrrolidine-1-carboxylate (301 mg, 0.36 mmol) in 20 mL of CH₂Cl₂ was passed O₃ at −78° C. until it turned blue. Excess of ozone was removed by blow N₂ through the solution. To the solution was added PPh₃ (609 mg, 2.32 mmol) and it was warmed to rt for 1.5 hr. Upon removal of volatiles, the crude aldehyde was purified by flash chromatography with EtOAc/hexanes=1:4. The aldehyde was dissolved in 4 mL of tBuOH and was added 1 mL of 2-methylbut-2-ene and a solution of NaClO₂ (357 mg, 3.96 mmol) and NaH₂PO₄ (412 mg, 2.99 mmol) in 1.6 mL of water. After 2 hr, the mixture was poured into CH₂Cl₂ and was washed with water. The organic phase was dried with Na₂SO₄, filtered and concentrated to afford the title compound.

### Step D: (1S,2R,8aR)-2-{(1R)-1-[3,5-bis(trifluoromethyl)phenyl]ethoxy}-1-(4-fluorophenyl)-5-oxooctahydroindolizin-8-yl acetate

The title compound was prepared from 4-(acetyloxy)-4-[(2R,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]butanoic acid with the same procedure as Step G in Example 1. The fast isomer on TLC plate with NH3-MeOH/EtOAc/Hexanes=1:10:10 was labeled isomer A and the slow isomer as isomer B. MS: 548 (M+1).

### Example 87 and Example 88

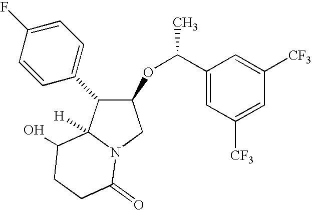

### (1S,2R,8aR)-2-{(1R)-1-[3,5-bis(trifluoromethyl)phenyl]ethoxy}-1-(4-fluorophenyl)-8-hydroxyhexahydroindolizin-5(1H)-one

A solution of the isomer A of 1S,2R,8aR)-2-{(1R)-1-[3,5-bis(trifluoromethyl)phenyl]ethoxy}-1-(4-fluorophenyl)-5-oxooctahydroindolizin-8-yl acetate (42 mg, 0.077) in 3 mL of methanol was added K₂CO₃ (21 mg, 0.15 mmol) and the solution was stirred at rt for 24 hr. Upon removal of volatiles, it was purified by prep TLC with MeOH/CH₂Cl₂=5:95 to afford the isomer A of the title compound. Similarly, the isomer B was prepared from isomer B of (1S,2R,8aR)-2-{(1R)-1-[3,5-bis(trifluoromethyl)phenyl]ethoxy}-1-(4-fluorophenyl)-5-oxooctahydroindolizin-8-yl acetate. MS: 506 (M+1).

### Example 89 and 90

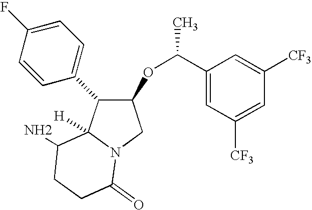

### (1S,2R,8aR)-8-amino-2-{(1R)-1-[3,5-bis(trifluoromethyl)phenyl]ethoxy}-1-(4-fluorophenyl)hexahydroindolizin-5(1H)-one

A solution of isomer A of (1S,2R,8aR)-2-{(1R)-1-[3,5-bis(trifluoromethyl)phenyl]ethoxy}-1-(4-fluorophenyl)-8-hydroxyhexahydroindolizin-5(1H)-one (58.2 mg, 0.11 mmol) in 3 mL of CH₂Cl₂ was added Et₃N (0.096 mL, 0.66 mmol) and methansulfonyl chloride (0.036 mL, 0.46 mmol) at 0° C. After 1 hr, it was diluted with ether and was washed with NaHCO₃. The organic phase was dried with Na₂SO₄, filtered and concentrated. The residue was redissolved in 3 mL of DMF and NaN3 (45 mg, 0.69 mmol) was added. The solution was heated at 66° C. for Ih hr. Upon removal of volatiles, the residue was dissolved in CH₂Cl₂ and was washed once with water. The organic phase was dried with Na₂SO₄, filtered and concentrated. The azide product was dissolved in 5 mL of MeOH and was added Pd—C (38 mg, 10%). The mixture was shaken under 35 psi of H₂ for 1.5 and was filtered. Upon removal of volatiles, the crude was purified by reverse phase HPLC to afford the title compound purified by prep TLC with NH₃-MeOH(2N)/CH₂Cl₂=4:96 to afford the isomer A of the title compounds.

The isomer B of the title compound was prepared from isomer B of (1S,2R,8aR)-2-{(1R)-1-[3,5-bis(trifluoromethyl)phenyl]ethoxy}-3-1-(4-fluorophenyl)-8-hydroxyhexahydroindolizin-5(1H)-one with the same procedure. MS: 506 (M+1).

### Example 91

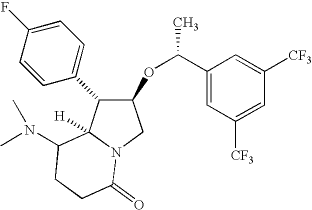

### (1S,2R,8aR)-2-{(1R)-1-[3,5-bis(trifluoromethyl)phenyl]ethoxy}-8-(dimethylamino)-1-(4-fluorophenyl)hexahydroindolizin-5(1H)-one

The title compound was prepared from the isomer B (1S,2R,8aR)-8-amino-2-{(1R)-1-[3,5-bis(trifluoromethyl)phenyl]ethoxy}-1-(4-fluorophenyl)hexahydroindolizin-5(1H)-one with the same procedure as Example 18. MS: 534 (M+1).

### Example 92

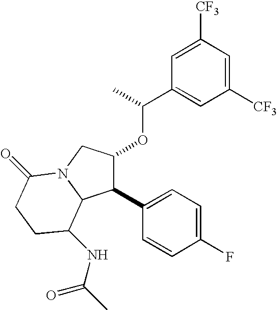

### N-[(1S,2R,8aR)-2-{(1R)-1-[3,5-bis(trifluoromethyl)phenyl]ethoxy}-1-(4-fluorophenyl)-5-oxooctahydroindolizin-8-yl]acetamide

The title compound was prepared from the isomer B (1S,2R,8aR)-8-amino-2-{(1R)-1-[3,5-bis(trifluoromethyl)phenyl]ethoxy}-1-(4-fluorophenyl)hexahydroindolizin-5(1H)-one with the same procedure as Example 19. MS: 547 (M+1).

### Example 93

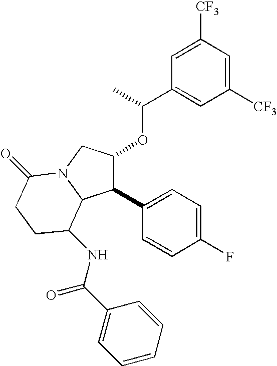

### N-[(1S,2R,8aR)-2-{(1R)-1-[3,5-bis(trifluoromethyl)phenyl]ethoxy}-1-(4-fluorophenyl)-5-oxooctahydroindolizin-8-yl]benzamide

The title compound was prepared from the isomer B (1S,2R,8aR)-8-amino-2-{(1R)-1-[3,5-bis(trifluoromethyl)phenyl]ethoxy}-1-(4-fluorophenyl)hexahydroindolizin-5(1H)-one with the same procedure as Example 19. MS: 609 (M+1).

### Example 94

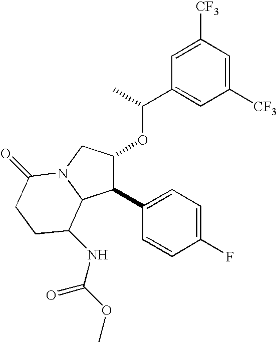

### Methyl [(1S,2R,8aR)-2-{(1R)-1-[3,5-bis(trifluoromethyl)phenyl]ethoxy}-1-(4-fluorophenyl)-5-oxooctahydroindolizin-8-yl]carbamate

A solution of the isomer B (1S,2R,8aR)-8-amino-2-{(1R)-1-[3,5-bis(trifluoromethyl)phenyl]ethoxy}-1-(4-fluorophenyl)hexahydroindolizin-5(1H)-one (10 mg, 0.020) in 3 mL of CH₂Cl₂ was added diisopropyl ethylamine (0.069 mL, 0.40 mmol) and methyl chloroformate (0.017 mL, 0.2 mmol) at rt and stirred for 2 hr. Upon removal of volatiles, it was purified by reverse phase HPLC to afford the title compound. MS: 563 (M+1).

### Example 95

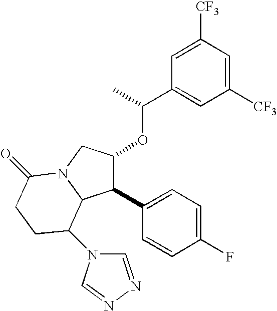

### (1S,2R,8aR)-2-{(1R)-1-[3,5-bis(trifluoromethyl)phenyl]ethoxy}-1-(4-fluorophenyl)-8-(4H-1,2,4-triazol-4-yl)hexahydroindolizin-5(1H)-one

The title compound was prepared from the isomer B (1S,2R,8aR)-8-amino-2-{(1R)-1-[3,5-bis(trifluoromethyl)phenyl]ethoxy}-1-(4-fluorophenyl)hexahydroindolizin-5(1H)-one with the same procedure as Example 29. MS: 557 (M+1).

### Example 96

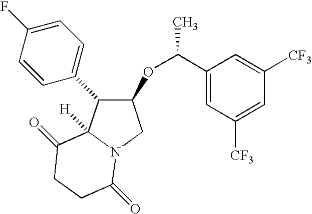

### (1S,2R,8aR)-2-{(1R)-1-[3,5-bis(trifluoromethyl)phenyl]ethoxy}-1-(4-fluorophenyl)hexahydroindolizine-5,8-dione

The title compound was prepared from the isomer B of (1S,2R,8aR)-2-{(1R)-1-[3,5-bis(trifluoromethyl)phenyl]ethoxy}-1-(4-fluorophenyl)-8-hydroxyhexahydroindolizin-5(1H)-one with the standard Swem oxidation condition (A. J. Mancuso and D. Swem, Synthesis, 1981, 165). MS: 504 (M+1).

### Example 97 and 98

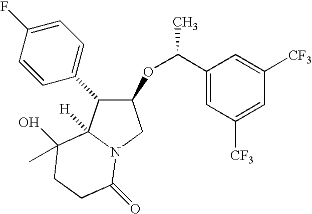

### (1S,2R,8aR)-2-{(1R)-1-[3,5-bis(trifluoromethyl)phenyl]ethoxy}-1-(4-fluorophenyl)-8-hydroxy-8-methylhexahydroindolizin-5(1H)-one

The title compounds were prepared from (1S,2R,8aR)-2-{(1R)-1-[3,5-bis(trifluoromethyl)phenyl]ethoxy}-1-(4-fluorophenyl)hexahydroindolizine-5,8-dione with the procedure as Example 35. MS: 520 (M+1).

### Example 99

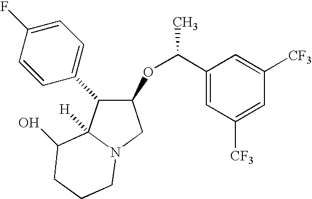

### (1S,2R,8aR)-2-{(1R)-1-[3,5-bis(trifluoromethyl)phenyl]ethoxy}-1-(4-fluorophenyl)-8-hydroxyhexahydroindolizin-5(1H)-one

The title compounds were prepared from the isomer B of (1S,2R,8aR)-2-{(1R)-1-[3,5-bis(trifluoromethyl)phenyl]ethoxy}-1-(4-fluorophenyl)-8-hydroxyhexahydroindolizin-5(1H)-one with the procedure as Example 73. MS: 520 (M+1).

### Example 100 and 101

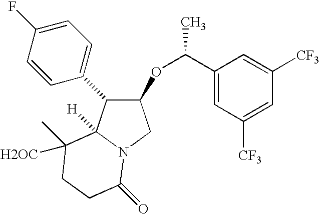

### (1S,2R,8aR)-2-{(1R)-1-[3,5-bis(trifluoromethyl)phenyl]ethoxy}-1-(4-fluorophenyl)-8-methyl-5-oxooctahydroindolizine-8-carboxylic acid

### Step A: (3S,4R)-4-{(1R)-1-[3,5-bis(trifluoromethyl)phenyl]ethoxy}-1-(tert-butoxycarbonyl)-3-(4-fluorophenyl)-D-proline

The title compound was prepared from tert-butyl 4-{1-[3,5-bis(trifluoromethyl)phenyl]ethoxy}-2-(hydroxymethyl)-3-phenylpyrrolidine-1-carboxylate with the swern oxidation followed by same procedure as step C in Example 85.

### Step B: tert-butyl (2S,3S,4R)-4-{(1R)-1-[3,5-bis(trifluoromethyl)phenyl]ethoxy}-3-(4-fluorophenyl)-2-(2-methoxy-2-oxoethyl)pyrrolidine-1-carboxylate

A solution of (3S,4R)-4-{(1R)-1-[3,5-bis(trifluoromethyl)phenyl]ethoxy}-1-(tert-butoxycarbonyl)-3-(4-fluorophenyl)-D-proline (1.93 g, 3.42 mmol) in 20 mL of THF was added Et₃N (0.81 mL, 5.81 mmol) and isobutyl chloroformate at 0° C. After 1 hr, fresh distilled CH2N2 in ether (generated from 5 g of N,4-dimethyl-N-nitrosobenzenesulfonamide) was added and stirred at 0° C. for 1 hr and at rt for 1 hr. It was quenched with HOAc and poured into ether. The organic phase was washed with NaHCO₃ and brine, dried with Na₂SO₄, filtered and concentrated. The crude was purified by flash chromatography with EtOAc/hexanes (10% to 100%) to afford the diazoketone intermediate, which was dissolved in 15 mL of methanol and was added silver benzoate (28 mg, 0.12 mmol) in 1 mL of Et₃N. After 18 hr, volatiles were removed and crude was purified by flash chromatography with EtOAc/hexanes (0% to 100%) to afford the title compound.

### Step C: tert-butyl (2R,3S,4R)-4-{(1R)-1-[3,5-bis(trifluoromethyl)phenyl]ethoxy}-3-(4-fluorophenyl)-2-(2-methoxy-1-methyl-2-oxoethyl)pyrrolidine-1-carboxylate

A solution of tert-butyl (2S,3S,4R)-4-{(1R)-1-[3,5-bis(trifluoromethyl)phenyl]ethoxy}-3-(4-fluorophenyl)-2-(2-methoxy-2-oxoethyl)pyrrolidine-1-carboxylate (0.24 g, 0.41 mmol) in 6 mL of THF was added LHMDS (3.29 mL, 3.29 mmol) at −78° C. and was added MeI after 20 minutes. It was allowed to warmed to −40° C. for 2 hr and was quenched with HOAc. It was poured into CH₂Cl₂ and was washed with NaHCO₃ and brine, dried with Na₂SO₄, filtered and concentrated. The crude was purified by prep TLC with acetone/hexanes=1:6 to afford the title compound.

### Step D: tert-butyl (2R,3S,4R)-4-{(1R)-1-[3,5-bis(trifluoromethyl)phenyl]ethoxy}-3-(4-fluorophenyl)-2-[1-(methoxycarbonyl)-1-methylbut-3-en-1-yl]pyrrolidine-1-carboxylate

The title compounds were prepared from tert-butyl (2R,3S,4R)-4-{(1R)-1-[3,5-bis(trifluoromethyl)phenyl]ethoxy}-3-(4-fluorophenyl)-2-(2-methoxy-1-methyl-2-oxoethyl)pyrrolidine-1-carboxylate and allyl iodide with the procedure as step C

### Step E: tert-butyl (2R,3S,4R)-4-{(1R)-1-[3,5-bis(trifluoromethyl)phenyl]ethoxy}-3-(4-fluorophenyl)-2-[4-hydroxy-1-(methoxycarbonyl)-1-methylbutyl]pyrrolidine-1-carboxylate

A solution of 2,3-dimethylbut-2-ene (0.047 mLg, 0.40 mmol) in 2 mL of THF was added BH₃.5Me₂ (0.20 mL, 0.40 mmol) at 0° C. After 40 minutes, a solution of tert-butyl (2R,3S,4R)-4-{(1R)-1-[3,5-bis(trifluoromethyl)phenyl]ethoxy}-3-(4-fluorophenyl)-2-[1-(methoxycarbonyl)-1-methylbut-3-en-1-yl]pyrrolidine-1-carboxylate and (0.027 mg, 0.050 mmol) in 3 mL of THF was added and was stirred at rt for 16 hr. It was cooled to 0° C. and was added a mixture of H₂O₂ and 2 N NaOH (3 mL, 1:1). The mixture was heated at 50° C. for 1 hr and was poured into EtOAc. The organic phase was washed with brine, dried with Na₂SO₄, filtered and concentrated. The crude was purified by prep TLC with acetone/hexanes=1:3 to afford the title compound.

### Step F: 4-[(2R,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]-5-methoxy-4-methyl-5-oxopentanoic acid

The title compound was prepared from tert-butyl (2R,3S,4R)-4-{(1R)-1-[3,5-bis(trifluoromethyl)phenyl]ethoxy}-3-(4-fluorophenyl)-2-[4-hydroxy-1-(methoxycarbonyl)-1-methylbutyl]pyrrolidine-1-carboxylate with the swem oxidation followed by same procedure as step C in Example 85.

### Step G: methyl (1S,2R,8aR)-2-{(1R)-1-[3,5-bis(trifluoromethyl)phenyl]ethoxy}-1-(4-fluorophenyl)-8-methyl-5-oxooctahydroindolizine-8-carboxylate

The title compound was prepared from 4-[(2R,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]-5-methoxy-4-methyl-5-oxopentanoic acid with the same procedure as step G in Example 1. MS: 562 (M+1).

### Step H: (1S,2R,8aR)-2-{(1R)-1-[3,5-bis(trifluoromethyl)phenyl]ethoxy}-1-(4-fluorophenyl)-8-methyl-5-oxooctahydroindolizine-8-carboxylic acid

The title compound was prepared from methyl (1,2R,8aR)-2-{(1R)-1-[3,5-bis(trifluoromethyl)phenyl]ethoxy}-1-(4-fluorophenyl)-8-methyl-5-oxooctahydroindolizine-8-carboxylate with the same procedure as Example 87. The fast isomer on reverse phase HPLC was labeled isomer A and the slow isomer as isomer B. MS: 548 (M+1).

### Example 102 and 103

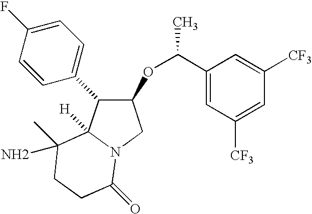

### (1S,2R,8aR)-8-amino-2-{(1R)-1-[3,5-bis(trifluoromethyl)phenyl]ethoxy}-1-(4-fluorophenyl)-8-methylhexahydroindolizin-5(1H)-one

The title compounds were prepared from 1S,2R,8aR)-2-{(1R)-1-[3,5-bis(trifluoromethyl)phenyl]ethoxy}-1-(4-fluorophenyl)-8-methyl-5-oxooctahydroindolizine-8-carboxylic acid with the same procedure as Step C and D in Example 8. MS: 519 (M+1).

### Example 104

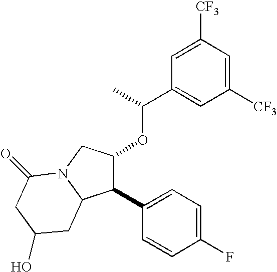

### (1S,2R,8aS)-2-{(1R)-1-[3,5-bis(trifluoromethyl)phenyl]ethoxy}-1-(4-fluorophenyl)-7-hydroxyhexahydroindolizin-5(1H)-one

### Step A: tert-butyl (2S,3S,4R)-4-{(1R)-1-[3,5-bis(trifluoromethyl)phenyl]ethoxy}-3-(4-fluorophenyl)-2-(2-oxoethyl)pyrrolidine-1-carboxylate

A solution of tert-butyl (2S,3S,4R)-4-{(1R)-1-[3,5-bis(trifluoromethyl)phenyl]ethoxy}-3-(4-fluorophenyl)-2-(2-methoxy-2-oxoethyl)pyrrolidine-1-carboxylate (Step B of Example 100) (1.95 g, 3.28 mmol) in 30 mL of CH₂Cl₂ was added DIBAL-H (5.25 mL, 5.25 mmol) at −78° C. After 1.5 hr, it was quenched with MeOH and warmed up to rt. The mixture was poured into ether and was added Na₂SO₄.10H₂O and stirred for 0.5 hr. It was filtered through celite and concentrated to afford the title compound.

### Step B: tert-butyl (2S,3S,4R)-4-{(1R)-1-[3,5-bis(trifluoromethyl)phenyl]ethoxy}-2-(4-tert-butoxy-2-hydroxy-4-oxobutyl)-3-(4-fluorophenyl)pyrrolidine-1-carboxylate

A solution of tert-butyl acetate (0.37 mL, 2.72 mmol) in 10 mL of THF was added LHMDS (2.41 mL, 2.41 mmol) at −78° C. After 15 minutes, a solution of tert-butyl (2S,3S,4R)-4-{(1R)-1-[3,5-bis(trifluoromethyl)phenyl]ethoxy}-3-(4-fluorophenyl)-2-(2-oxoethyl)pyrrolidine-1-carboxylate (0.85 g, 1.51 mmol) in 5 mL of THF was added at −78° C. After 1 hr, it was warmed to −40° C. for 1 hr. It was quenched with brine and warmed up to rt. The mixture was poured into ether and was washed with NaHCO₃, dried with Na₂SO₄, filtered and concentrated. The crude was purified by flash chromatography with EtOAc/hexanes=1:6 to 1:4 to provide the title compounds. MS: 524 (M−155).

### Step C: (1S,2R,8aS)-2-{(1R)-1-[3,5-bis(trifluoromethyl)phenyl]ethoxy}-1-(4-fluorophenyl)-7-hydroxyhexahydroindolizin-5(1H)-one

The title compound was prepared tert-butyl (2S,3S,4R)-4-{(1R)-1-[3,5-bis(trifluoromethyl)phenyl]ethoxy}-2-(4-tert-butoxy-2-hydroxy-4-oxobutyl)-3-(4-fluorophenyl)pyrrolidine-1-carboxylate with the same procedure as step G of Example 1. MS: 506 (M+1).

### Example 105

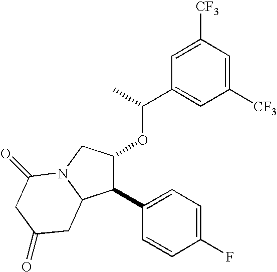

### (1S,2R,8aS)-2-{(1R)-1-[3,5-bis(trifluoromethyl)phenyl]ethoxy}-1-(4-fluorophenyl)tetrahydroindolizine-5,7(1H,6H)-dione

A solution of (1S,2R,8aS)-2-{(1R)-1-[3,5-bis(trifluoromethyl)phenyl]ethoxy}-1-(4-fluorophenyl)-7-hydroxyhexahydroindolizin-5(1H)-one (0.089 g; 0.18 mmol) in 5 mL of CH₂Cl₂ was added PCC-Alumina (0.38 g, 0.35 mmol) at rt. After 18 hr, it was filtered through celite. The crude was purified by prep TLC with EtOAc/hexanes=2:1 to afford the title compound. MS: 504 (M+1).

### Example 106

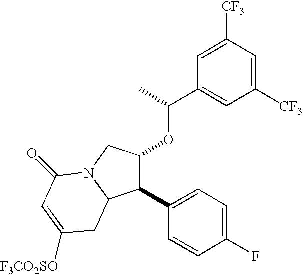

### (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl trifluoromethanesulfonate

A solution of (1S,2R,8aS)-2-{(1R)-1-[3,5-bis(trifluoromethyl)phenyl]ethoxy}-1-(4-fluorophenyl)tetrahydroindolizine-5,7(1H,6H)-dione (0.31 g, 0.62 mmol) in 6 mL of THF was added KHMDS (1.85 mL, 0.925 mmol) at −78° C. After 0.5 hr, it was added a solution of N-(5-chloropyridin-2-yl)-1,1,1-trifluoro-N-[(trifluoromethyl)sulfonyl]inethanesulfonamide (0.303 g, 0.77 mmol) in 3 mL of THF at −78° C. After 1.5 hr, It was poured into ether and was washed with NaHCO₃ and brine, dried with Na₂SO₄, filtered and concentrated. The crude was purified by flash chromatography with EtOAc/hexanes=1:4 to afford the title compound. MS: 636 (M+1).

### Example 107

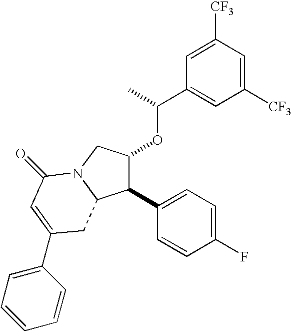

### (1S,2R,8aS)-2-{(1R)-1-[3,5-bis(trifluoromethyl)phenyl]ethoxy}-1-(4-fluorophenyl)-7-phenyl-2,3,8,8a-tetrahydroindolizin-5(1H)-one

A solution of (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl trifluoromethanesulfonate (0.023 g, 0.039 mmol), PhB(OH)₂ (0.096 g, 0.078 mmol), Na₂CO₃ (0.0083, 0.078 mmol), Pd(PPh₃)₄ (0.0091 g, 0.0078 mmol) in 1.5 mL of toluene was added 0.25 mL of water and 0.25 mL of ethanol. The solution was heated at 120° C. for 16 hr and was poured into ether. It was washed with NaHCO₃, dried with Na₂SO₄, filtered and concentrated. The crude was purified by prep TLC with EtOAc/hexanes=1:1 to afford the title compound. MS: 564 (M+1).

### Example 108

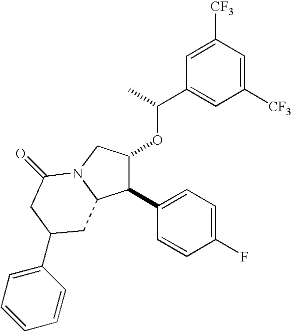

### (1S,2R,8aS)-2-{(1R)-1-[3,5-bis(trifluoromethyl)phenyl]ethoxy}-1-(4-fluorophenyl)-7-phenylhexahydroindolizin-5(1H)-one

The title compound was prepared from (1S,2R,8aS)-2-{(1R)-1-[3,5-bis(trifluoromethyl)phenyl]ethoxy}-1-(4-fluorophenyl)-7-phenyl-2,3,8,8a-tetrahydroindolizin-5(1H)-one with the same procedure as Step C of Example 1. MS: 566 (M+1).

### Example 109

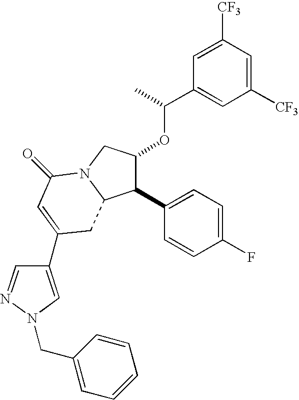

### (1S,2R,8aS)-7-(1-benzyl-1H-pyrazol-4-yl)-2-{(1R)-1-[3,5-bis(trifluoromethyl)phenyl]ethoxy}-1-(4-fluorophenyl)-2,3,8,8a-tetrahydroindolizin-5(1H)-one. MS: 644 (M+1).

### Example 110

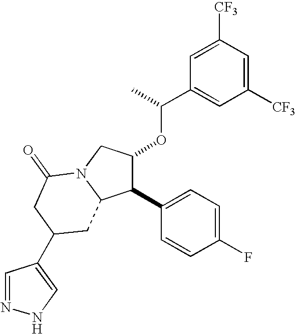

### (1S,2R,8aS)-2-{(1R)-1-[3,5-bis(trifluoromethyl)phenyl]ethoxy}-1-(4-fluorophenyl)-7-(1H-pyrazol-4-yl)hexahydroindolizin-5(1H)-one

The title compound was prepared from (1S,2R,8aS)-7-(1-benzyl-1H-pyrazol-4-yl)-2-{(1R)-1-[3,5-bis(trifluoromethyl)phenyl]ethoxy}-1-(4-fluorophenyl)-2,3,8,8a-tetrahydroindolizin-5(1H)-one with the same procedure as Step C of Example 1. MS: 556 (M+1).

### Example 111

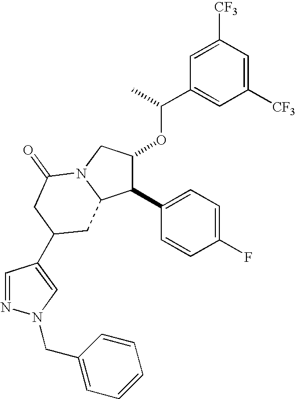

### (1S,2R,8aS)-7-(1-benzyl-1H-pyrazol-4-yl)-2-{(1R)-1-[3,5-bis(trifluoromethyl)phenyl]ethoxy}-1-(4-fluorophenyl)hexahydroindolizin-5(1H)-one

The title compound was isolated as side product in example. MS: 646 (M+1).

### Example 112

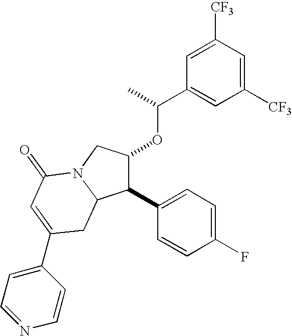

### (1S,2R,8aS)-2-{(1R)-1-[3,5-bis(trifluoromethyl)phenyl]ethoxy}-1-(4-fluorophenyl)-7-pyridin-4-yl-2,3,8,8a-tetrahydroindolizin-5(1H)-one

MS: 565 (M+1).

### Example 113 and Example 114

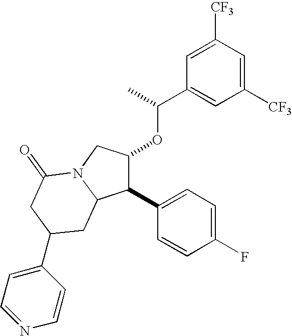

### (1S,2R,8aS)-2-{(1R)-1-[3,5-bis(trifluoromethyl)phenyl]ethoxy}-1-(4-fluorophenyl)-7-pyridin-4-ylhexahydroindolizin-5(1H)-one

MS: 567 (M+1).

### Example 115

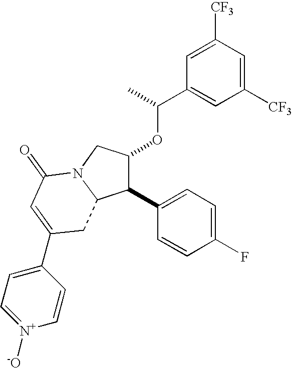

### (1S,2R,8aS)-2-{(1R)-1-[3,5-bis(trifluoromethyl)phenyl]ethoxy}-1-(4-fluorophenyl)-7-(1-oxidopyridin-4-yl)-2,3,8,8a-tetrahydroindolizin-5(1H)-one

A solution of (1S,2R,8aS)-2-{(1R)-1-[3,5-bis(trifluoromethyl)phenyl]ethoxy}-1-(4-fluorophenyl)-7-pyridin-4-yl-2,3,8,8a-tetrahydroindolizin-5(1H)-one (0.010 g, 0.016 mmol) in 2 mL of CH₂Cl₂ was added 0.25 MCPBA (16 mg, 0.071 mmol). After 1 hr, It was poured into CH₂Cl₂. It was washed with 2 N NaOH, dried with Na₂SO₄, filtered and concentrated to afford the title compound. MS: 581 (M+1).

### Example 116

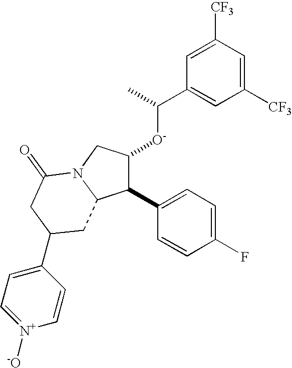

### (1S,2R,8aS)-2-{(1R)-1-[3,5-bis(trifluoromethyl)phenyl]ethoxy}-1-(4-fluorophenyl)-7-(1-oxidopyridin-4-yl)hexahydroindolizin-5(1H)-one

MS: 583 (M+1).

### Example 117

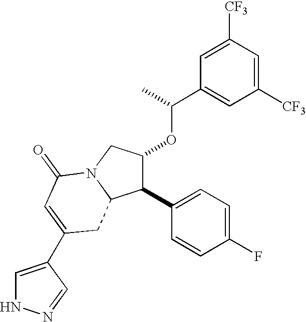

### (1S,2R,8aS)-2-{(1R)-1-[3,5-bis(trifluoromethyl)phenyl]ethoxy}-1-(4-fluorophenyl)-7-(1H-pyrazol-4-yl)-2,3,8,8a-tetrahydroindolizin-5(1H)-one

MS: 554 (M+1).

### Example 118

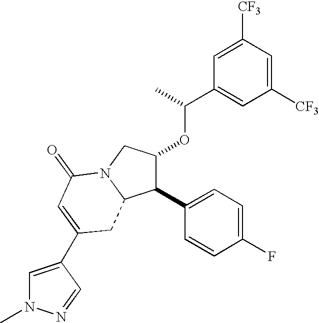

### (1S,2R,8aS)-2-{(1R)-1-[3,5-bis(trifluoromethyl)phenyl]ethoxy}-1-(4-fluorophenyl)-7-(1-methyl-1H-pyrazol-4-yl)-2,3,8,8a-tetrahydroindolizin-5(1H)-one

MS: 568 (M+1).

### Example 119

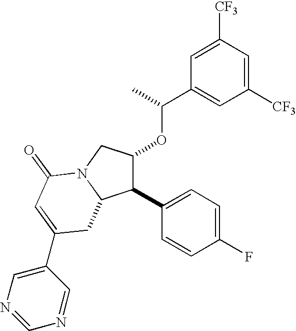

### (1S,2R,8aS)-2-{(R)-1-[3,5-bis(trifluoromethyl)phenyl]ethoxy}-1-(4-fluorophenyl)-7-pyrimidin-5-yl-2,3,8,8a-tetrahydroindolizin-5(1H)-one

MS: 566 (N+1).

### Example 120

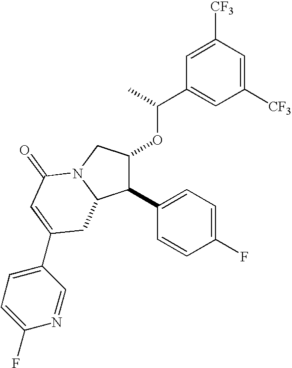

### (1S,2R,8aS)-2-{(1R)-1-[3,5-bis(trifluoromethyl)phenyl]ethoxy}-1-(4-fluorophenyl)-7-(6-fluoropyridin-3-yl)-2,3,8,8a-tetrahydroindolizin-5(1H)-one

MS: 583 (M+1).

### Example 121

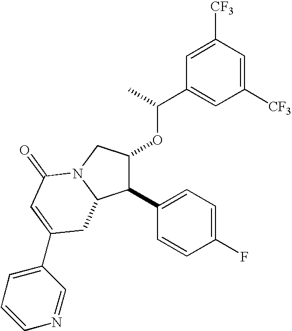

### (1S,2R,8aS)-2-{(1R)-1-[3,5-bis(trifluoromethyl)phenyl]ethoxy}-1-(4-fluorophenyl)-7-pyridin-3-yl-2,3,8,8a-tetrahydroindolizin-5(1H)-one

MS: 565 (M+1).

### Example 122

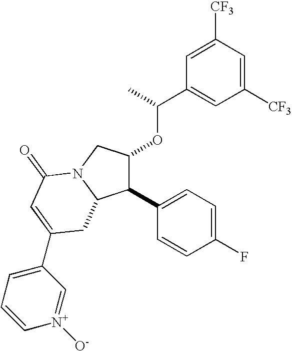

### (1S,2R,8aS)-2-{(1R)-1-[3,5-bis(trifluoromethyl)phenyl]ethoxy}-1-(4-fluorophenyl)-7-(1-oxidopyridin-3-yl)-2,3,8,8a-tetrahydroindolizin-5(1H)-one

MS: 581 (M+1).

### Example 123

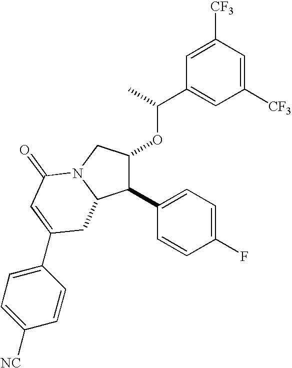

### 4-[(1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl]benzonitrile

MS: 589 (M+1).

### Example 124

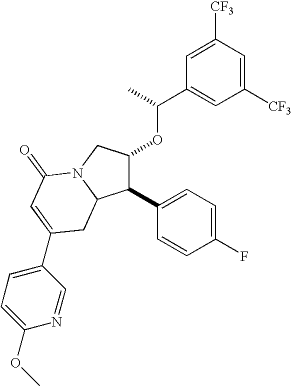

### (1S,2R,8aS)-2-{(1R)-1-[3,5-bis(trifluoromethyl)phenyl]ethoxy}-1-(4-fluorophenyl)-7-(6-methoxypyridin-3-yl)-2,3,8,8a-tetrahydroindolizin-5(1H)-one

MS: 595 (M+1).

### Example 125

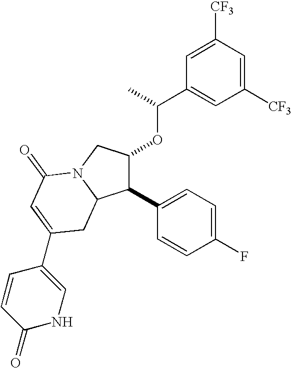

A solution of (1S,2R,8aS)-2-{(1R)-1-[3,5-bis(trifluoromethyl)phenyl]ethoxy}-1-(4-fluorophenyl)-7-(6-methoxypyridin-3-yl)-2,3,8,8a-tetrahydroindolizin-5(1H)-one (0.012 g, 0.020 mmol) in 3 mL of chloroform was added TMSI (0.029 mL, 0.2 mmol) and the solution was heated at reflux for 4.5 hr. Then 1 mL of methanol was added and the resulting solution was heated at reflux for 1 hr. Upon removal of volatiles, the crude was purified by reverse phase HPLC to afford the title compound. MS: 581 (M+1).

### Example 126

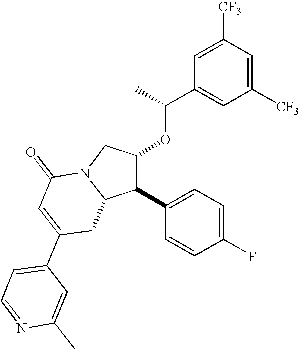

### (1S,2R,8aS)-2-{(1R)-1-[3,5-bis(trifluoromethyl)phenyl]ethoxy}-1-(4-fluorophenyl)-7-(2-methylpyridin-4-yl)-2,3,8,8a-tetrahydroindolizin-5(1H)-one

MS: 579 (M+1).

### Example 127

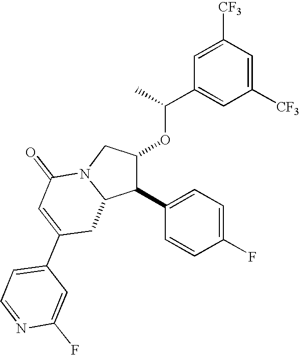

### (1S,2R,8aS)-2-{(1R)-1-[3,5-bis(trifluoromethyl)phenyl]ethoxy}-1-(4-fluorophenyl)-7-(2-fluoropyridin-4-yl)-2,3,8,8a-tetrahydroindolizin-5(1H)-one

MS: 583 (M+1).

### Example 128 and 129

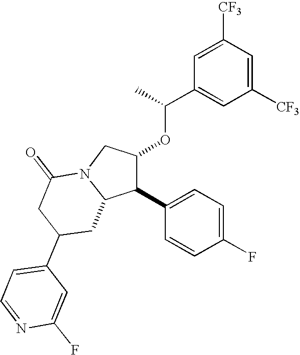

### (1S,2R,8aS)-2-{(1R)-1-[3,5-bis(trifluoromethyl)phenyl]ethoxy}-1-(4-fluorophenyl)-7-(2-fluoropyridin-4-yl)hexahydroindolizin-5(1H)-one

MS: 585 (M+1).

### Example 130

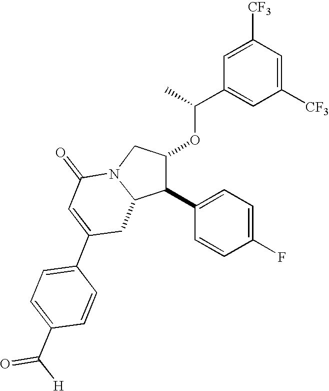

### 4-[(1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl]benzaldehyde

MS: 592 (M+1).

### Example 131

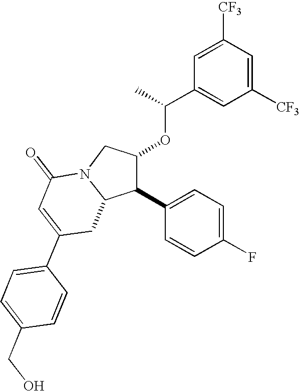

The title compound was prepared from (4-[(1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl]benzaldehyde with the same procedure as Example 33.

MS: 594 (M+1).

### Example 132

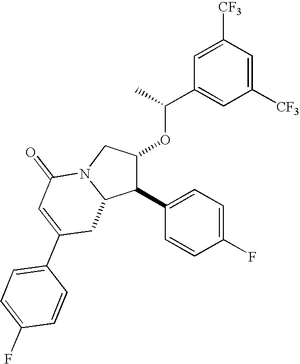

### (1S,2R,8aS)-2-{(1R)-1-[3,5-bis(trifluoromethyl)phenyl]ethoxy}-1,7-bis(4-fluorophenyl)-2,3,8,8a-tetrahydroindolizin-5(1H)-one

MS: 582 (N+1).

### Example 133

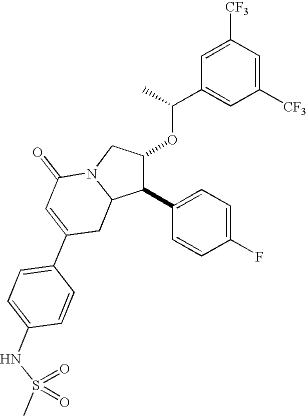

### N-{4-[(1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl]phenyl}methanesulfonamide

MS: 657 (M+1).

### Example 134

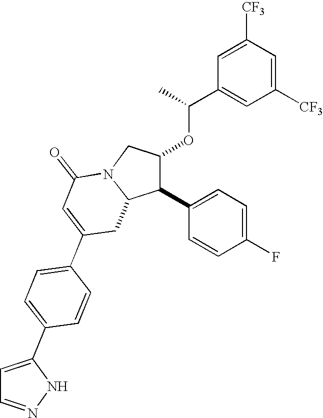

### (1S,2R,8aS)-2-{(1R)-1-[3,5-bis(trifluoromethyl)phenyl]ethoxy}-1-(4-fluorophenyl)-7-[4-(1H-pyrazol-5-yl)phenyl]-2,3,8,8a-tetrahydroindolizin-5(1H)-one

MS: 630 (M+1).

### Example 135

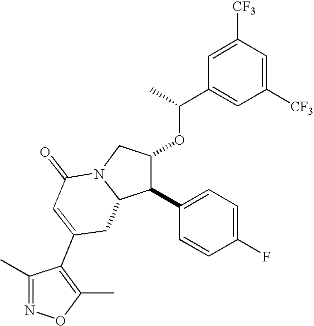

### (1S,2R,8aS)-2-{(1R)-1-[3,5-bis(trifluoromethyl)phenyl]ethoxy}-7-(3,5-dimethylisoxazol-4-yl)-1-(4-fluorophenyl)-2,3,8,8a-tetrahydroindolizin-5(1H)-one

MS: 583 (M+1).

### Example 136

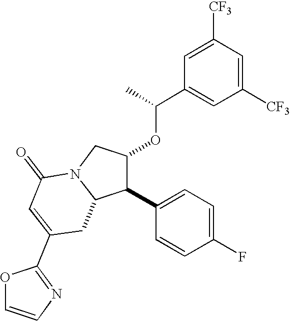

### (1S,2R,8aS)-2-{(1R)-1-[3,5-bis(trifluoromethyl)phenyl]ethoxy}-1-(4-fluorophenyl)-7-(1,3-oxazol-2-yl)-2,3,8,8a-tetrahydroindolizin-5(1H)-one

MS: 555 (M+1).

### Example 137

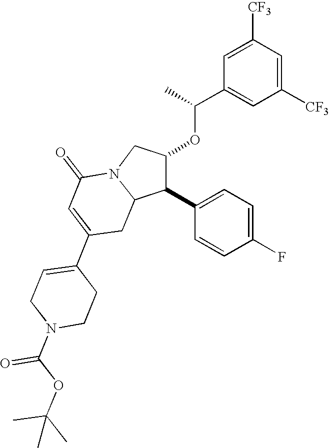

### Tert-butyl 4-[(1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl]-3,6-dihydropyridine-1(2H)-carboxylate

MS: 669 (M+1).

### Example 138

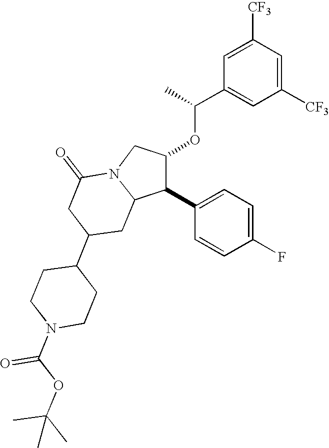

### Tert-butyl 4-[(1S,2R,8aS)-2-{(1R)-1-[3,5-bis(trifluoromethyl)phenyl]ethoxy}-1-(4-fluorophenyl)-5-oxooctahydroindolizin-7-yl]piperidine-1-carboxylate

MS: 617 (M-55).

### Example 139

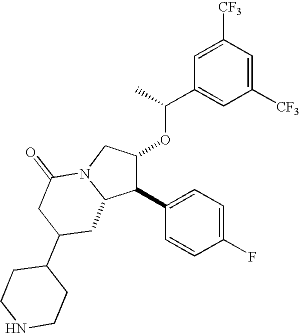

### (1S,2R,8aS)-2-{(1R)-1-[3,5-bis(trifluoromethyl)phenyl]ethoxy}-1-(4-fluorophenyl)-7-piperidin-4-ylhexahydroindolizin-5(1H)-one

The title compound was prepared from (tert-butyl 4-[(1S,2R,8aS)-2-{(1R)-1-[3,5-bis(trifluoromethyl)phenyl]ethoxy}-1-(4-fluorophenyl)-5-oxooctahydroindolizin-7-yl]piperidine-1-carboxylate with the same procedure as Step G of Example 1. MS: 573 (M+1).

### Example 140

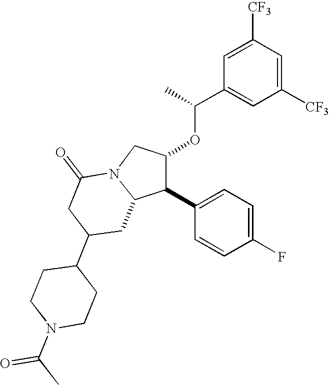

### (1S,2R,8aS)-7-(1-acetylpiperidin-4-yl)-2-{(1R)-1-[3,5-bis(trifluoromethyl)phenyl]ethoxy}-1-(4-fluorophenyl)hexahydroindolizin-5(1H)-one

The title compound was prepared from (1S,2R,8aS)-2-{(1R)-1-[3,5-bis(trifluoromethyl)phenyl]ethoxy}-1-(4-fluorophenyl)-7-piperidin-4-ylhexahydroindolizin-5(1H)-one with the same procedure as Step G of Example 19. MS: MS: 615 (M+1).

### Example 141

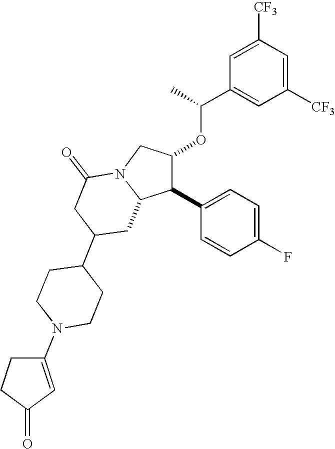

### (1S,2R,8aS)-2-{(1R)-1-[3,5-bis(trifluoromethyl)phenyl]ethoxy}-1-(4-fluorophenyl)-7-[1-(3-oxocyclopent-1-en-1-yl)piperidin-4-yl]hexahydroindolizin-5(1H)-one

The title compound was prepared from (1S,2R,8aS)-2-{(1R)-1-[3,5-bis(trifluoromethyl)phenyl]ethoxy}-1-(4-fluorophenyl)-7-piperidin-4-ylhexahydroindolizin-5(1H)-one with the same procedure as Step G of Example 27. MS: 653 (M+1).

### Example 142

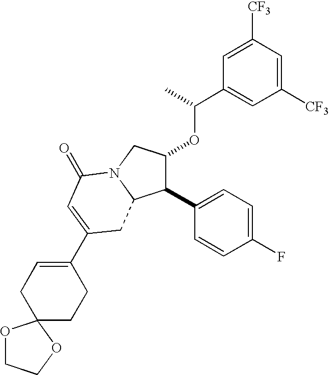

### (1S,2R,8aS)-2-{(1R)-1-[3,5-bis(trifluoromethyl)phenyl]ethoxy}-7-(1,4-dioxaspiro[4.5]dec-7-en-8-yl)-1-(4-fluorophenyl)-2,3,8,8a-tetrahydroindolizin-5(1H)-one MS: 626 (M+1).

### Example 143

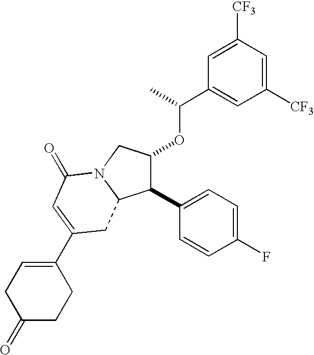

### (1S,2R,8aS)-2-{(1R)-1-[3,5-bis(trifluoromethyl)phenyl]ethoxy}-1-(4-fluorophenyl)-7-(4-oxocyclohex-1-en-1-yl)-2,3,8,8a-tetrahydroindolizin-5(1H)-one

A solution of (1S,2R,8aS)-2-{(1R)-1-[3,5-bis(trifluoromethyl)phenyl]ethoxy}-7-(1,4-dioxaspiro[4.5]dec-7-en-8-yl)-1-(4-fluorophenyl)-2,3,8,8a-tetrahydroindolizin-5(1H)-one (0.030 g, 0.048 mmol) in 2 mL of acetone was added PPTS (3 mg, 0.012 mmol) and 0.2 mL of water. The solution was heated at reflux for 20 hr. Upon removal of volatiles, the crude was purified by prep TLC with acetone/hexanes=1:3 to afford the title compound. MS: 582 (M+1).

### Example 144

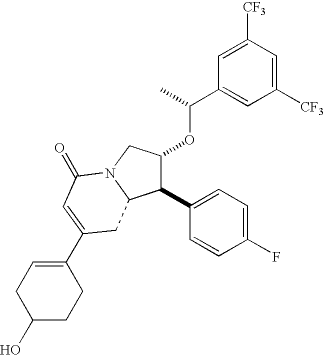

### (1S,2R,8aS)-2-{(1R)-1-[3,5-bis(trifluoromethyl)phenyl]ethoxy}-1-(4-fluorophenyl)-7-(4-hydroxycyclohex-1-en-1-yl)-2,3,8,8a-tetrahydroindolizin-5(1H)-one

The title compound was prepared from (1S,2R,8aS)-2-{(1R)-1-[3,5-bis(trifluoromethyl)phenyl]ethoxy}-1-(4-fluorophenyl)-7-(4-oxocyclohex-1-en-1-yl)-2,3,8,8a-tetrahydroindolizin-5(1H)-one with the same procedure as Example 33. MS: 584 (M+1).

### Example 145

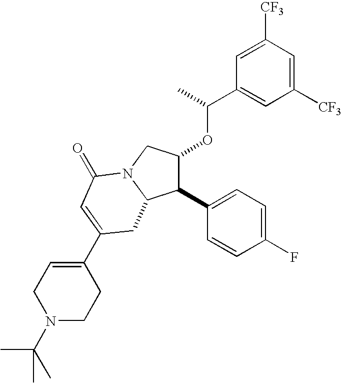

### (1S,2R,8aS)-2-{(1R)-1-[3,5-bis(trifluoromethyl)phenyl]ethoxy}-7-(1-tert-butyl-1,2,3,6-tetrahydropyridin-4-yl)-1-(4-fluorophenyl)-2,3,8,8a-tetrahydroindolizin-5(1H)-one

A solution of (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl trifluoromethanesulfonate (0.021 g, 0.036 mmol), 1-tert-butyl-4-(trimethylstannyl)-1,2,3,6-tetrahydropyridine (0.022 g, 0.073 mmol), Pd(PPh₃)₄ (0.0084 g, 0.0072 mmol) and LiCl (4.2 mg, 0.15 mmol) in 3 mL of dioxane was heated under N2 at 120° C. for 16 hr. It was poured into CH₂Cl₂. It was washed with NaHCO₃, dried with Na₂SO₄, filtered and concentrated. The crude was purified by reverse phase afford the title compound. MS: 625 (M+1).

### Example 146

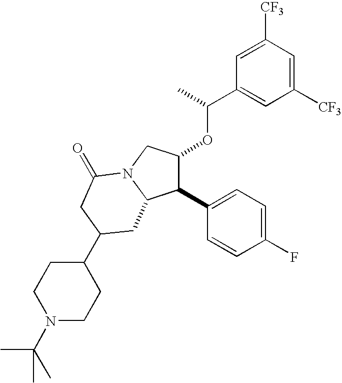

### (1S,2R,8aS)-2-{(1R)-1-[3,5-bis(trifluoromethyl)phenyl]ethoxy}-7-(1-tert-butylpiperidin-4-yl) 1-(4-fluorophenyl)hexahydroindolizin-5(1H)-one

To a solution of (1S,2R,8aS)-2-{(1R)-1-[3,5-bis(trifluoromethyl)phenyl]ethoxy}-7-(1-tert-butyl-1,2,3,6-tetrahydropyridin-4-yl)-1-(4-fluorophenyl)-2,3,8,8a-tetrahydroindolizin-5(1H)-one (8.2 mg, 0.013 mmol) in 3 mL methanol under nitrogen atmosphere was added 10 mg 10% Pd—C catalyst. The resulting mixture was stirred under 50 psi of hydrogen at RT. After 2 hours, the catalyst was filtered through filter-aid and the solvent was removed under vacuum to afford the title compound. MS: 629 (M+1).

### Example 146

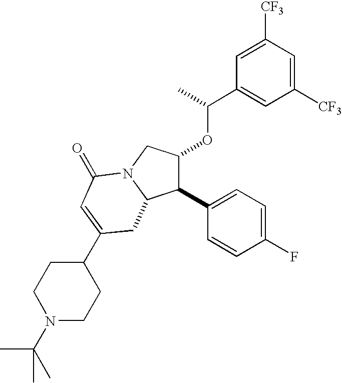

### (1S,2R,8aS)-2-{(1R)-1-[3,5-bis(trifluoromethyl)phenyl]ethoxy}-7-(1-tert-butylpiperidin-4-yl)-1-(4-fluorophenyl)-2,3,8,8a-tetrahydroindolizin-5(1H)-one

To a solution of (1S,2R,8aS)-2-{(1R)-1-[3,5-bis(trifluoromethyl)phenyl]ethoxy}-7-(1-tert-butyl-1,2,3,6-tetrahydropyridin-4-yl)-1-(4-fluorophenyl)-2,3,8,8a-tetrahydroindolizin-5(1H)-one (74.9 mg, 0.12 mmol) in 10 mL methanol under nitrogen atmosphere was added 10 mg 10% Pd—C catalyst. The resulting mixture was stirred under 25 psi of hydrogen at RT. After 16 hours, the catalyst was filtered through filter-aid and the solvent was removed under vacuum. The crude was purified by prep TLC with NH3-MeOH/EtOAc/hexanes=1:10:10 to afford the title compound. MS: 627 (M+1).

### Example 147

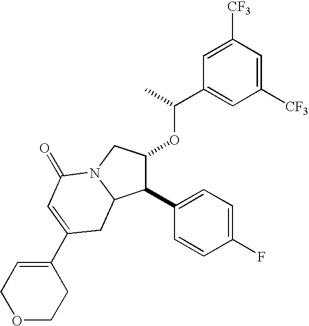

### (1s,2R,8aS)-2-{(1R)-1-[3,5-bis(trifluoromethyl)phenyl]ethoxy}-7-(3,6-dihydro-2H-pyran-4-yl)-1-(4-fluorophenyl)-2,3,8,8a-tetrahydroindolizin-5(1H)-one

The title compound was prepared from (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl trifluoromethanesulfonate with the same procedure as Example 145. MS: 570 (M+1).

### Example 148 and 149

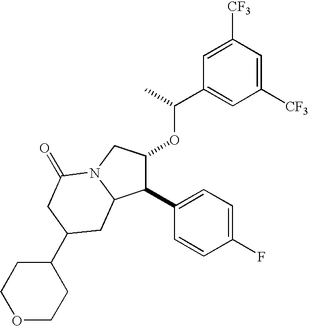

### (1S,2R,8aS)-2-{(1R)-1-[3,5-bis(trifluoromethyl)phenyl]ethoxy}-1-(4-fluorophenyl)-7-(tetrahydro-2H-pyran-4-yl)hexahydroindolizin-5(1H)-one

The title compound was prepared from (1S,2R,8aS)-2-{(1R)-1-[3,5-bis(trifluoromethyl)phenyl]ethoxy}-7-(3,6-dihydro-2H-pyran-4-yl)-1-(4-fluorophenyl)-2,3,8,8a-tetrahydroindolizin-5(1H)-one with the same procedure as Example 146. MS: 574 (M+1).

### Example 150

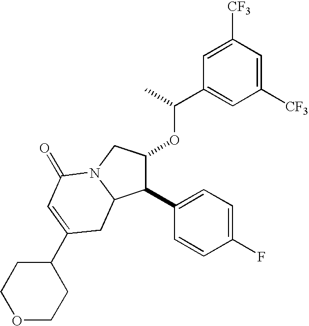

### (1S,2R,8aS)-2-{(1R)-1-[3,5-bis(trifluoromethyl)phenyl]ethoxy}-1-(4-fluorophenyl)-7-(tetrahydro-2H-pyran-4-yl)-2,3,8,8a-tetrahydroindolizin-5(1H)-one

The title compound was prepared from (1S,2R,8aS)-2-{(1R)-1-[3,5-bis(trifluoromethyl)phenyl]ethoxy}-7-(3,6-dihydro-2H-pyran-4-yl)-1-(4-fluorophenyl)-2,3,8,8a-tetrahydroindolizin-5(1H)-one with the same procedure as Example 146 except that the reaction time is 15 minutes. MS: 572 (M+1).

### Example 151 and 152

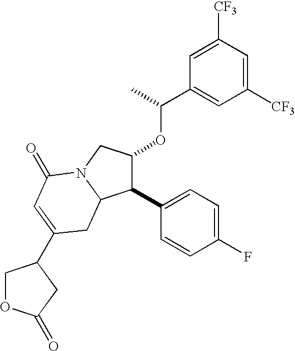

### (1S,2R,8aS)-2-{(1R)-1-[3,5-bis(trifluoromethyl)phenyl]ethoxy}-1-(4-fluorophenyl)-7-(5-oxotetrahydrofuran-3-yl)-2,3,8,8a-tetrahydroindolizin-5(1H)-one

A solution of (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl trifluoromethanesulfonate (0.030 g, 0.047 mmol), (2Z)-but-2-ene-1,4-diol (0.005 mL, 0.061 mmol), NaHCO₃ (0.01 g, 0.12 mmol), tetrabutylammonium chloride (13 mg, 0.046 mmol) and Pd(OAc)₂ (0.4 mg, 0.0018 mmol) in 1 mL of DMF was heated under N₂ at 70° C. for 3 hr. It was poured into EtOAc. It was washed with water, dried with Na₂SO₄, filtered and concentrated. The intermediate was dissolved in 3 mL toluene and was added Ag2CO3-celite (55 mg, 0.1 mmol). The mixture was heated at 80° C. for 24 hr and was filtered. Upon removal of volatiles, crude was purified by prep TLC to afford the title compounds. MS: 572 (M+1).

### Example 153

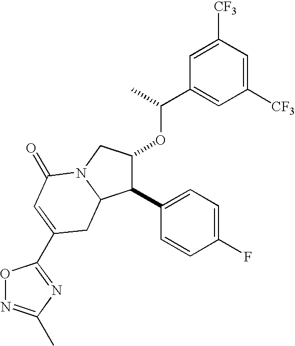

### (1S,2R,8aS)-2-{(1R)-1-[3,5-bis(trifluoromethyl)phenyl]ethoxy}-1-(4-fluorophenyl)-7-(3-methyl-1,2,4-oxadiazol-5-yl)-2,3,8,8a-tetrahydroindolizin-5(1H)-one

A solution of (1S,2R,8aS)-2-{(1R)-1-[3,5-bis(trifluoromethyl)phenyl]ethoxy}-1-(4-fluorophenyl)-5-oxo-1,2,3,5,8,8a-hexahydroindolizin-7-yl trifluoromethanesulfonate (0.020 g, 0.034 mmol), methylamidoxime (15 mg, 0.20 mmol), Et₃N (0.1 mL, 0.72 mmol) and Pd(PPh₃)₄ (7.9 mg, 0.068 mmol) in 3 mL of toluene was heated under CO balloon at 95° C. for 16 hr. Upon removal of volatiles, the crude was purified by prep TLC with MeOH/CH₂Cl₂=5:95 afford the title compound. MS: 570 (M+1).

### Example 154 and Example 155

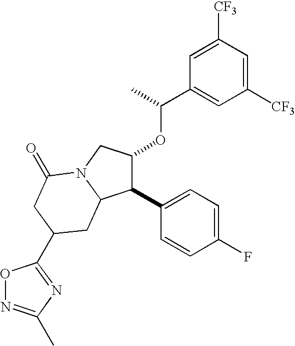

### (1S,2R,8aS)-2-{(1R)-1-[3,5-bis(trifluoromethyl)phenyl]ethoxy}-7-(3-methyl-1,2,4-oxadiazol-5-yl)-1-(4-fluorophenyl)hexahydroindolizin-5(1H)-one

MS: 572 (M+1).

### Example 156

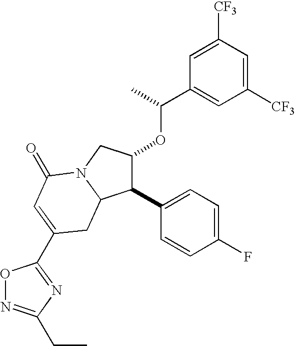

### (1S,2R,8aS)-2-{(1R)-1-[3,5-bis(trifluoromethyl)phenyl]ethoxy}-7-(3-ethyl-1,2,4-oxadiazol-5-yl)-1-(4-fluorophenyl)-2,3,8,8a-tetrahydroindolizin-5(1H)-one

MS: 584 (M+1).

### Example 157

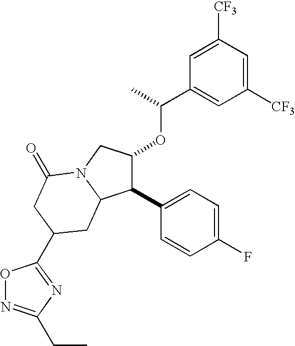

### (1S,2R,8aS)-2-{(1R)-1-[3,5-bis(trifluoromethyl)phenyl]ethoxy}-7-(3-ethyl-1,2,4-oxadiazol-5-yl)-1-(4-fluorophenyl)hexahydroindolizin-5(H)-one

MS: 586 (M+1).

### Example 158

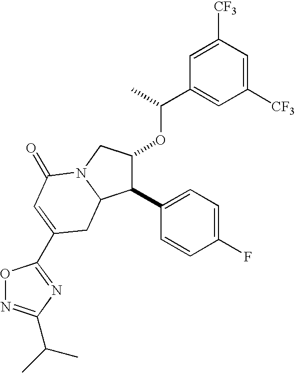

### (1S,2R,8aS)-2-{(1R)-1-[3,5-bis(trifluoromethyl)phenyl]ethoxy}-1-(4-fluorophenyl)-7-(3-isopropyl-1,2,4-oxadiazol-5-yl)-2,3,8,8a-tetrahydroindolizin-5(1H)-one

MS: 598 (M+1).

### Example 159

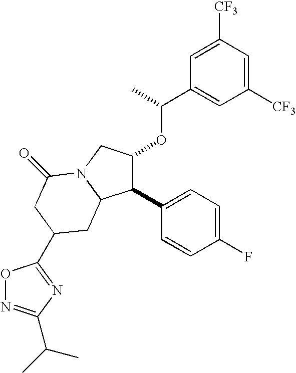

### (1S,2R,8aS)-2-{(1R)-1-[3,5-bis(trifluoromethyl)phenyl]ethoxy}-1-(4-fluorophenyl)-7-(3-isopropyl-1,2,4-oxadiazol-5-yl)hexahydroindolizin-5(1H)-one

MS: 600 (M+1).

### Example 160

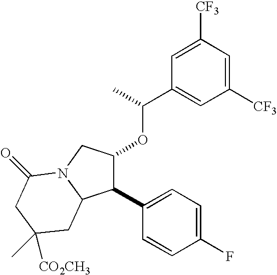

### Methyl (1S,2R,8aS)-2-{(1R)-1-[3,5-bis(trifluoromethyl)phenyl]ethoxy}-1-(4-fluorophenyl)-7-methyl-5-oxooctahydroindolizine-7-carboxylate

### Step A: tert-butyl (2S,3S,4R)-4-{(1R)-1-[3,5-bis(trifluoromethyl)phenyl]ethoxy}-3-(4-fluorophenyl)-2-(3-methoxy-2-methyl-3-oxopropyl)pyrrolidine-1-carboxylate

A solution of tert-butyl (2S,3S,4R)-4-{(1R)-1-[3,5-bis(trifluoromethyl)phenyl]ethoxy}-3-(4-fluorophenyl)-2-(3-methoxy-3-oxopropyl)pyrrolidine-1-carboxylate (Step C of Example 1) (0.12 g, 0.20 mmol) in 5 mL of THF was added LHMDS (1.58 mL, 1.58 mmol) at −78° C. and was added MeI after 20 minutes. It was stirred at −78° C. for 2 hr and was quenched with HOAc. It was poured into ether and was washed with NaHCO3 and brine, dried with Na₂SO₄, filtered and concentrated to afford the title compound.

### Step B: tert-butyl (2S,3S,4R)-4-{(1R)-1-[3,5-bis(trifluoromethyl)phenyl]ethoxy}-3-(4-fluorophenyl)-2-[2-(methoxycarbonyl)-2-methylpent-4-en-1-yl]pyrrolidine-1-carboxylate

A solution of tert-butyl (2S,3S,4R)-4-{(1R)-1-[3,5-bis(trifluoromethyl)phenyl]ethoxy}-3-(4-fluorophenyl)-2-(3-methoxy-2-methyl-3-oxopropyl)pyrrolidine-1-carboxylate (0.25 g, 0.40 mmol) in 15 mL of THF was added LHMDS (1.58 mL, 1.58 mmol) at −78° C. and was added allyl iodide after 20 minutes. It was warmed to rt for 80 minutes and was quenched with HOAc. It was poured into ether and was washed with NaHCO₃ and brine, dried with Na₂SO₄, filtered and concentrated. The crude was purified by flash chromatography with EtOAc/hexanes=1:9 to afford the title compound.

### Step C: 3-{[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]methyl}-4-methoxy-3-methyl-4-oxobutanoic acid

The title compound was prepared from tert-butyl (2S,3S,4R)-4-{(1R)-1-[3,5-bis(trifluoromethyl)phenyl]ethoxy}-3-(4-fluorophenyl)-2-[2-(methoxycarbonyl)-2-methylpent-4-en-1-yl]pyrrolidine-1-carboxylate same procedure as step C in Example 85. MS: 580 (M-99).

### Step D: methyl (1S,2R,8aS)-2-{(1R)-1-[3,5-bis(trifluoromethyl)phenyl]ethoxy}-1-(4-fluorophenyl)-7-methyl-5-oxooctahydroindolizine-7-carboxylate

The title compound was prepared from 3-{[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]methyl}-4-methoxy-3-methyl-4-oxobutanoic acid with the same procedure as step G in Example 1. MS: 562 (M+1).

### Example 161

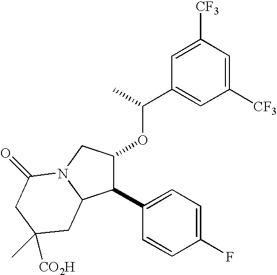

### (1S,2R,8aS)-2-{(1R)-1-[3,5-bis(trifluoromethyl)phenyl]ethoxy}-1-(4-fluorophenyl)-7-methyl-5-oxooctahydroindolizine-7-carboxylic acid

A solution of methyl (1S,2R,8aS)-2-{(1R)-1-[3,5-bis(trifluoromethyl)phenyl]ethoxy}-1-(4-fluorophenyl)-7-methyl-5-oxooctahydroindolizine-7-carboxylate (71 mg, 0.13) and LiOH.H₂O (26 mg, 0.63 mmol) in 1 mL of methanol was 1 mL of THF and 1 mL of water. The solution was heated at 60° C. for 16 hr and was poured into CH₂Cl₂. The organic phase was washed with pH=4 buffer solution. The aqueous phase was extracted with CH₂Cl₂ (2×) and EtOAc (1×). The combined organic phase was dried with Na₂SO₄, filtered and concentrated to afford the title compound. MS: 548 (M+1).

### Example 162

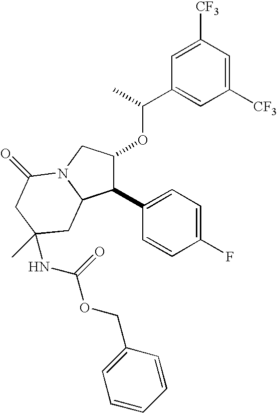

### Benzyl [(1S,2R,8aS)-2-{(1R)-1-[3,5-bis(trifluoromethyl)phenyl]ethoxy}-1-(4-fluorophenyl)-7-methyl-5-oxooctahydroindolizin-7-yl]carbamate

The title compound was prepared from (1S,2R,8aS)-2-{(1R)-1-[3,5-bis(trifluoromethyl)phenyl]ethoxy}-1-(4-fluorophenyl)-7-methyl-5-oxooctahydroindolizine-7-carboxylic acid with the same procedure as step C of Example 8. MS: 653 (M+1).

### Example 163

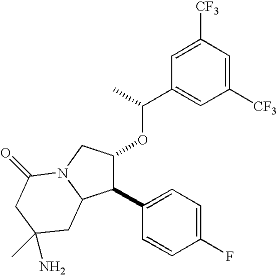

### (1S,2R,8aS)-7-amino-2-{(1R)-1-[3,5-bis(trifluoromethyl)phenyl]ethoxy}-1-(4-fluorophenyl)-7-methylhexahydroindolizin-5(1H)-one

The title compound was prepared from benzyl [(1S,2R,8aS)-2-{(1R)-1-[3,5-bis(trifluoromethyl)phenyl]ethoxy}-1-(4-fluorophenyl)-7-methyl-5-oxooctahydroindolizin-7-yl]carbamate with the same procedure as step D of Example 8. MS: 519 (M+1).

### Example 164

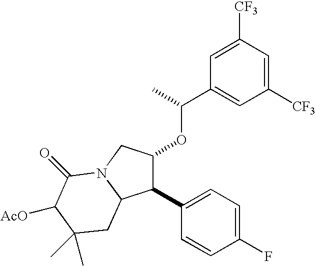

### (1S,2R,8aS)-2-{(1R)-1-[3,5-bis(trifluoromethyl)phenyl]ethoxy}-1-(4-fluorophenyl)-7,7-dimethyl-5-oxooctahydroindolizin-6-yl acetate

### Step A: tert-butyl (2S,3S,4R)-4-{(1R)-1-[3,5-bis(trifluoromethyl)phenyl]ethoxy}-3-(4-fluorophenyl)-2-(3-methoxy-2,2-dimethyl-3-oxopropyl)pyrrolidine-1-carboxylate

A solution of tert-butyl (2S,3S,4R)-4-{(1R)-1-[3,5-bis(trifluoromethyl)phenyl]ethoxy}-3-(4-fluorophenyl)-2-(3-methoxy-2-methyl-3-oxopropyl)pyrrolidine-1-carboxylate (0.115 g, 0.19 mmol) in 5 mL of THF was added LHMDS (1.58 mL, 1.58 mmol) at −78° C. and was added MeI (0.074 mL, 1.19 mmol) after 20 minutes. It was warmed to rt for 90 minutes and was quenched with NH₄Cl. It was poured into ether and was washed with NaHCO₃ and brine, dried with Na₂SO₄, filtered and concentrated to afford the title compound.

### Step B: tert-butyl (2S,3S,4R)-4-{(1R)-1-[3,5-bis(trifluoromethyl)phenyl]ethoxy}-3-(4-fluorophenyl)-2-(3-hydroxy-2,2-dimethylpropyl)pyrrolidine-1-carboxylate

A solution of tert-butyl (2S,3S,4R)-4-{(1R)-1-[3,5-bis(trifluoromethyl)phenyl]ethoxy}-3-(4-fluorophenyl)-2-(3-methoxy-2,2-dimethyl-3-oxopropyl)pyrrolidine-1-carboxylate (0.158 g, 0.25 mmol) in 5 mL of CH₂Cl₂ was added DIBAL-H (0.52 mL, 0.52 mmol) at −78° C. After 110 minutes it was quenched with MeOH. It was poured into CH₂Cl₂ and was dried with Na₂SO₄, filtered and concentrated to afford the title compound.

### Step C: tert-butyl (2S,3S,4R)-4-{(1R)-1-[3,5-bis(trifluoromethyl)phenyl]ethoxy}-2-(2,2-dimethyl-3-oxopropyl)-3-(4-fluorophenyl)pyrrolidine-1-carboxylate

The title compound was prepared from tert-butyl (2S,3S,4R)-4-{(1R)-1-[3,5-bis(trifluoromethyl)phenyl]ethoxy}-3-(4-fluorophenyl)-2-(3-hydroxy-2,2-dimethylpropyl)pyrrolidine-1-carboxylate with the same procedure as step A in Example 1. MS: (M+1).

### Step D: tert-butyl (2S,3S,4R)-2-[3-(acetyloxy)-2,2-dimethylpent-4-en-1-yl]-4-{(1R)-1-[3,5-bis(trifluoromethyl)phenyl]ethoxy}-3-(4-fluorophenyl)pyrrolidine-1-carboxylate

The title compound was prepared tert-butyl (2S,3S,4R)-4-{(1R)-1-[3,5-bis(trifluoromethyl)phenyl]ethoxy}-2-(2,2-dimethyl-3-oxopropyl)-3-(4-fluorophenyl)pyrrolidine-1-carboxylate same procedure as Example 35 followed by acylation by the procedure in example 19.

### Step E: 2-(acetyloxy)-4-[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]-3,3-dimethylbutanoic acid

The title compound was prepared from tert-butyl (2S,3S,4R)-2-[3-(acetyloxy)-2,2-dimethylpent-4-en-1-yl]-4-{(R)-1-[3,5-bis(trifluoromethyl)phenyl]ethoxy}-3-(4-fluorophenyl)pyrrolidine-1-carboxylate by same procedure as Step C in Example 85. MS: 694 (M+1)

### Step F: (1S,2R,8aS)-2-{(1R)-1-[3,5-bis(trifluoromethyl)phenyl]ethoxy}-1-(4-fluorophenyl)-7,7-dimethyl-5-oxooctahydroindolizin-6-yl acetate

The title compound was prepared from 2-(acetyloxy)-4-[(2S,3S,4R)-4-{(1R)-1-[3,5-bis(trifluoromethyl)phenyl]ethoxy}-1-(tert-butoxycarbonyl)-3-(4-fluorophenyl)pyrrolidin-2-yl]-3,3-dimethylbutanoic acid by same procedure as Step G in Example 1. MS: 576 (M+1).

### Example 165 and 166

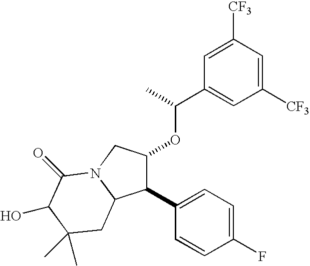

### (1S,2R,8aS)-2-{(1R)-1-[3,5-bis(trifluoromethyl)phenyl]ethoxy}-1-(4-fluorophenyl)-6-hydroxy-7,7-dimethylhexahydroindolizin-5(1H)-one

The title compound was prepared from (1S,2R,8aS)-2-{(1R)-1-[3,5-bis(trifluoromethyl)phenyl]ethoxy}-1-(4-fluorophenyl)-7,7-dimethyl-5-oxooctahydroindolizin-6-yl acetate by same procedure as in Example 87. MS: 534 (M+1).

What is claimed is:

1. A compound of the formula I: 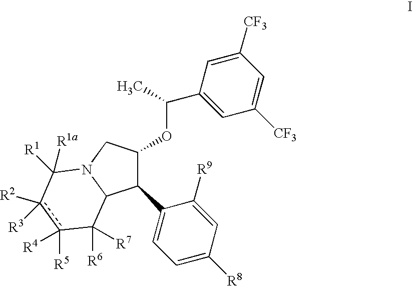
wherein:
R¹ and R^{1a} are each hydrogen or together with the carbon atom to which they are attached form a carbonyl;
R² is selected from the group consisting of:
(1) hydrogen, and
(2) CH₃;
R³ are each independently selected from the group consisting of:
(1) hydrogen,
(2) hydroxyl,
(3) NH₂,
(4) C₁₋₆alkyl,
(5) hydroxyC₁₋₆alkyl,
(6) C₁₋₁₆alkyl-O—C₁₋₆alkyl,
(7) N(CH₃)₂;
(8) NH—C(O)—C(CH₃)₂—NH₂,
(9) NH—C(O)—CF₃,
(10) -A1, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocycle is optionally substituted with a group selected from methyl, oxo and hydroxyl,
(11) —NH-A1
(12) —NH—CH₂-A1,
(13) —O—C(O)—CH₃,
(14) CO₂Me; and
(15) CO₂H;
R⁴ is selected from hydrogen and methyl;
R⁵ is selected from a group consisting of:
(1) hydrogen,
(2) hydroxy,
(3) hydroxyC₁₋₃alkyl,
(4) C₁₋₄alkyl,
(5) C₁₋₄alkyl-NH₂,
(6) C₁₋₄alkyl-NH—C(O)CH₃,
(7) C₁₋₄alkyl-NH—C(O)CH₂Cl,
(8) C₁₋₄alkyl-NH—C(O)H,
(9) —C(O)—O—CH₃,
(10) —C(O)—CH₃,
(11) —O—S(O)₂—CF₃,
(12) —C(O)—OH,
(13) —C(O)—N(R¹⁰)(R¹¹),
(14) phenyl, optionally substituted with a substituent selected from the group consisting of halo, methyl, hydroxyc 14alkyl, —C(O)H and —NH—S(O)₂—CH₃,
(15) —NH—S(O)₂—CH₃,
(16) —NH-cyclopentenone,
(17) —NH-cyclohexenone, optionally substituted with a substituent selected from halo, hydroxy and oxo,
(18) —NH—C(O)—C₁₋₄alkyl,
(19) —NH—C(O)-phenyl, optionally substituted with halo, —C(O)H or cyano,
(20) —NH—C(O)-pyridyl, optionally substituted with halo, —C(O)H or cyano,
(21) —NH—C(O)—NH-phenyl, optionally substituted with halo, —C(O)H or cyano,
(22) —NH—C(O)—NH-pyridyl, optionally substituted with halo, —C(O)H or cyano,
(23) —NH—C(O)—O—CH₂—C₂₋₄alkenyl,
(24) —NH—C₁₋₄alkyl,
(25) —NH—C(O)—C₁₋₄alkyl-NH₂,
(26) —NH—C(O)—C₁₋₄alkyl-NH₂—C(O)—O—CH₂-phenyl,
(27) —N(R¹⁰)(R¹¹),
(28) A2, wherein A2 is selected from the group consisting of 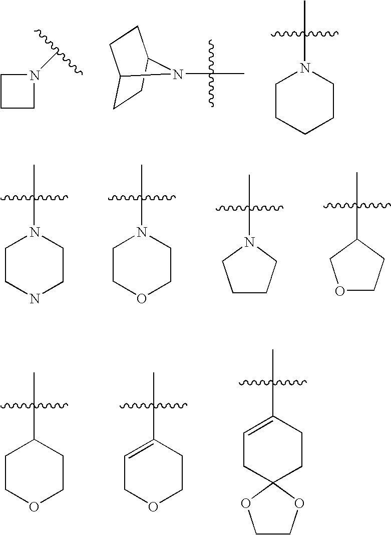
wherein A2 is optionally substituted with one or two groups selected from halo, hydroxyl, cyano, C₁₋₄alkyl, NH₂, COOH, oxo, —COO—C₁₋₄alkyl, —NH—C(O)—CH₂Cl, —C(O)CH₃, and 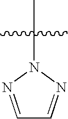
(29) A3, wherein A3 is a heteraromatic ring of 5 or 6 atoms or N-oxide thereof, wherein 1, 2, or 3 of the atoms is a heteroatom selected from N, S or O, and wherein at least one of the heteroatoms is a N, and wherein the heteroaryl or heterocycle is optionally substituted with one or two groups selected from halo, cyano, C₁₋₄alkyl, oxo, hydroxyl, phenyl, benzyl, —OCH₃, —CH₃—NH₂, —NH—C(O)—CH₃Cl, and —CH₃—N(CH₃)₂,
(30) CH₃-A2,
(31) CH₃-A3,
(32) —NH-A2,
(33) —C(O)-A2,
(34) —NH-A3,
(35) —C(O)-A3,
or R⁴ and R⁵ together with the carbon to which they are attached form a carbonyl;
R⁶ is selected from hydrogen and methyl,
R⁷ are each independently selected from a group consisting of:
(1) hydrogen,
(2) halo, and
(3) hydroxyl, and
(4) methyl,
(5) —C(O)OH,
(6) —O—C(O)—CH₃,
(7) NH—C(O)—CH₃,
(8) NH—C(O)-phenyl,
(9) NH—C(O)—O—CH₃,
(10) NH₂, and
(11) A4, wherein A4 is a heteroaryl or N-oxide thereof or heterocycle of 5 or 6 atoms wherein the heteroaryl or heterocyle contains 1, 2 or 3 heteroatoms selected from the group consisting of O, N and S, and wherein the heteroaryl or heterocycle is optionally substituted with a group selected from methyl, oxo, hydroxyl, —CH₃—NH₂ and CH₃—N(CH₃)₂;
or R⁶ and R⁷ together with the carbon to which they are attached form a carbonyl;
R⁸ and R⁹ are each independently selected from a group consisting of:
(1) hydrogen,
(2) halo, and
(3) methyl;
R¹⁰ ad R¹¹ are each selected from the group consisting of
(1) hydrogen,
(2) methyl,
(3) —O—CH₃
(4) A5, wherein A5 is a heteroaryl or N-oxide thereof or heterocycle of 5 or 6 atoms wherein the heteroaryl or heterocyle contains 1, 2 or 3 heteroatoms selected from the group consisting of O, N and S, and wherein the heteroaryl or heterocycle is optionally substituted with a group selected from methyl, oxo, hydroxyl, —CH₃—NH₂ and —CH₃—N(CH₃)₂, and
(5) —C₁₋₃alkyl-A5,
or a pharmaceutically acceptable salts thereof and individual enantiomers and diastereomers thereof.

2. A compound according to claim 1 wherein
A1 is selected from the group consisting of

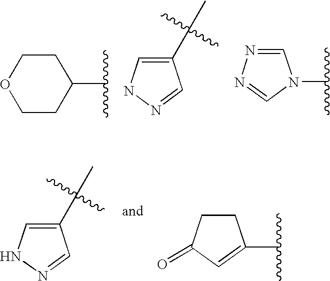

3. A compound according to claim 1 wherein
R³ is selected from the group consisting of
(1) NH₂, and
(2) —NH-A1, wherein A1 is selected from the group consisting of 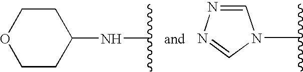
and A1 is optionally substituted with a group selected from methyl, oxo and hydroxyl.

4. A compound according to claim 1 wherein A3 is selected from the group consisting of 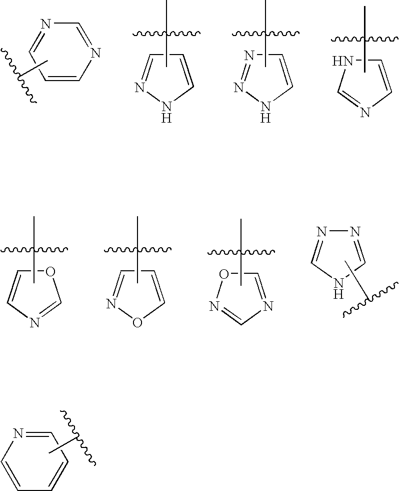
or an N-oxide thereof wherein A3 is optionally substituted with one or two substituents selected from the group consisting of halo, cyano, C₁₋₄alkyl, oxo, hydroxyl, phenyl, benzyl, —OCH₃, —CH₃—NH₂, —NH—C(O)—CH₃Cl, and CH₃—N(CH₃)₂.

5. A compound according to claim 1 wherein A4 is selected from the group consisting of 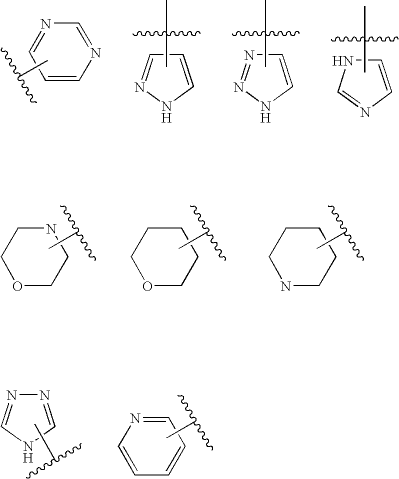
wherein A4 is optionally substituted with one or two substituents selected from the group consisting of hydroxylmethyl, oxo, hydroxyl, —CH₃—NH₂ and —CH₃—N(CH₃)₂.

6. A compound according to claim 1 wherein
R⁵ is selected from a group consisting of:
(1) hydroxy,
(2) NH₂, and
(3) N(R¹⁰)(R¹¹).

7. A compound according to claim 1 wherein
R⁷ is selected from a group consisting of:
(1) hydrogen,
(2) fluoro, and
(3) methyl.

8. A compound according to claim 1 wherein
R⁸ and R⁹ are each independently selected from a group consisting of:
(1) hydrogen,
(2) fluoro, and
(3) methyl.

9. A compound according to claim 1 wherein
R¹⁰ ad R¹¹ are each selected from the group consisting of
(1) hydrogen, and
(2) -A4, wherein A4 is selected from the group consisting of 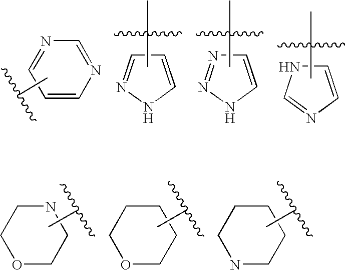
wherein A4 is optionally substituted with a substituent selected from the group consisting of hydroxyl, oxo, methyl, —CH₃—NH₂ and —CH₃—N(CH₃)₂.

10. A compound according to claim 1 wherein
R⁸ is fluoro and R⁹ is methyl or hydrogen.

11. A compound according to claim 1 of the formula

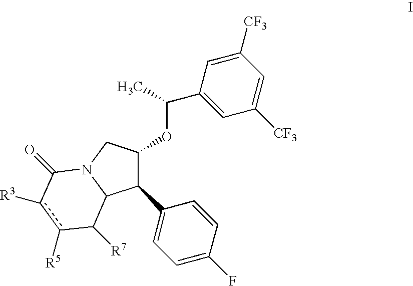

12. A compound according to claim 1 wherein
R¹ and R^{1a} are each hydrogen or together with the carbon atom to which they are attached form a carbonyl;
R² is selected from the group consisting of:
(1) hydrogen, and
(2) CH₃;
R³ is selected from the group consisting of
(1) NH₂, and
(2) —NH-A1, wherein A1 is selected from the group consisting of 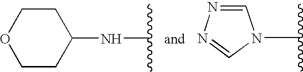
and A1 is optionally substituted with a group selected from methyl, oxo and hydroxyl;
R⁴ is selected from hydrogen and methyl;
R⁵ is selected from a group consisting of:
(1) hydroxy,
(2) NH₂, and
(3) N(R¹⁰)(R¹¹);
R⁶ is selected from hydrogen and methyl,
R⁷ is selected from a group consisting of:
(1) hydrogen,
(2) fluoro, and
(3) methyl.
R⁸ is fluoro and R⁹ is methyl or hydrogen.
R¹⁰ ad R¹¹ are each selected from the group consisting of
(1) hydrogen, and
(2) -A4, wherein A4 is selected from the group consisting of 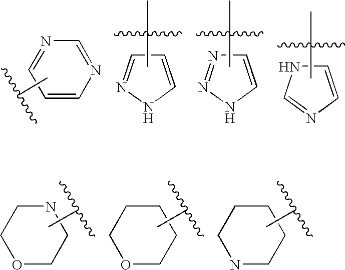
wherein A4 is optionally substituted with a substituent selected from the group consisting of hydroxyl, oxo, methyl, —CH₃—NH₂ and CH₃—N(CH₃)₂;
or a pharmaceutically acceptable salts thereof and individual enantiomers and diastereomers thereof.

13. A compound which is selected from the group consisting of: 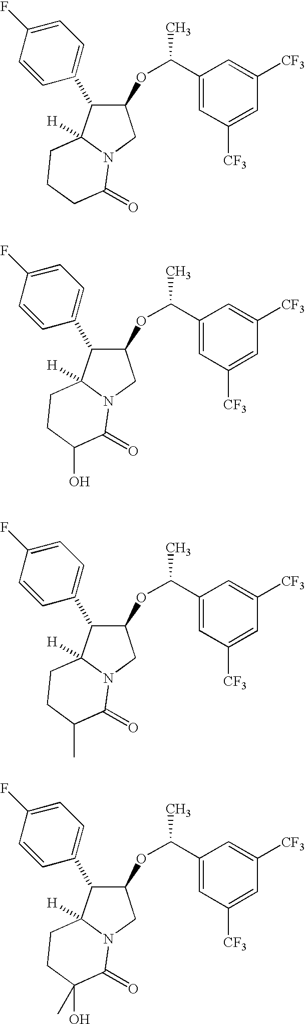 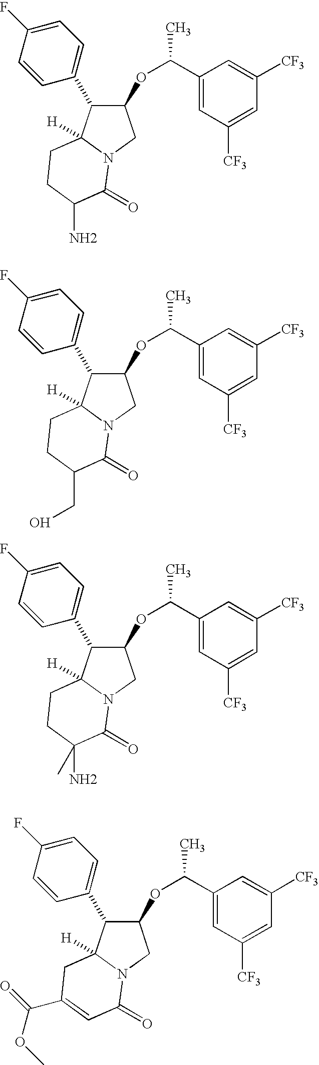 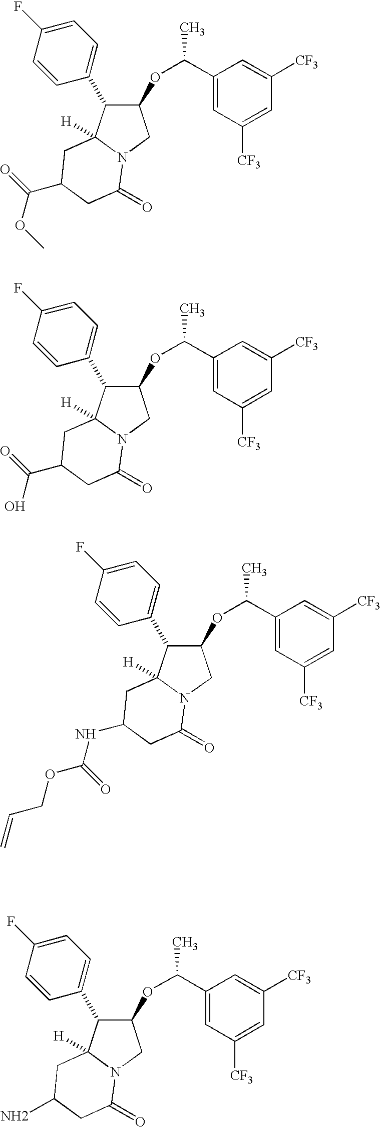 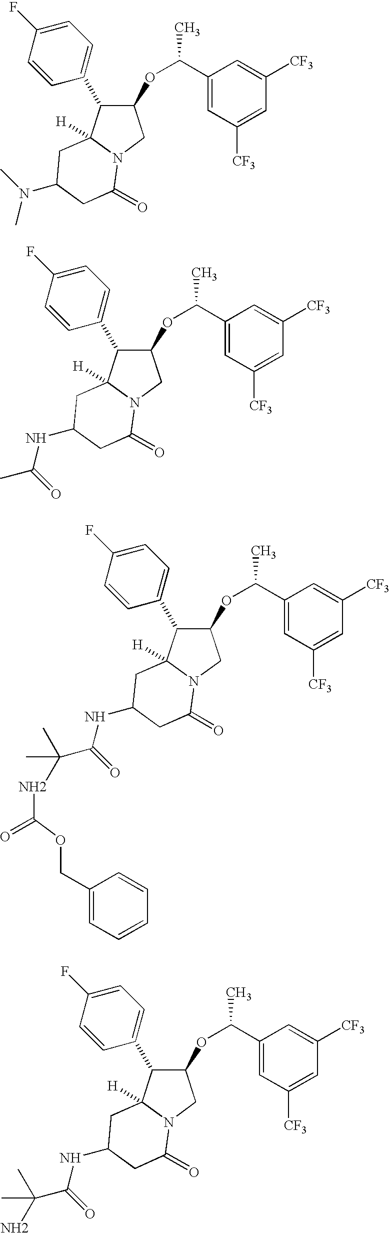 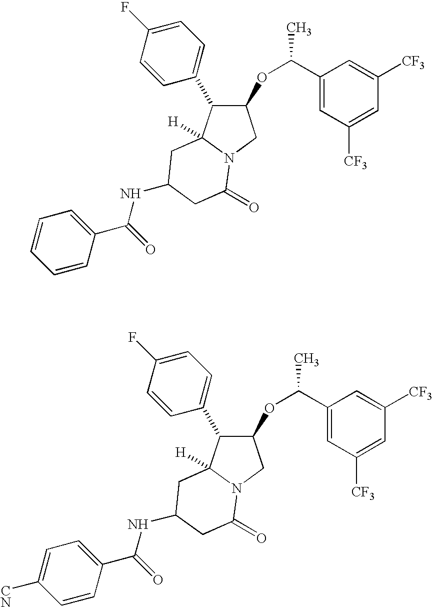 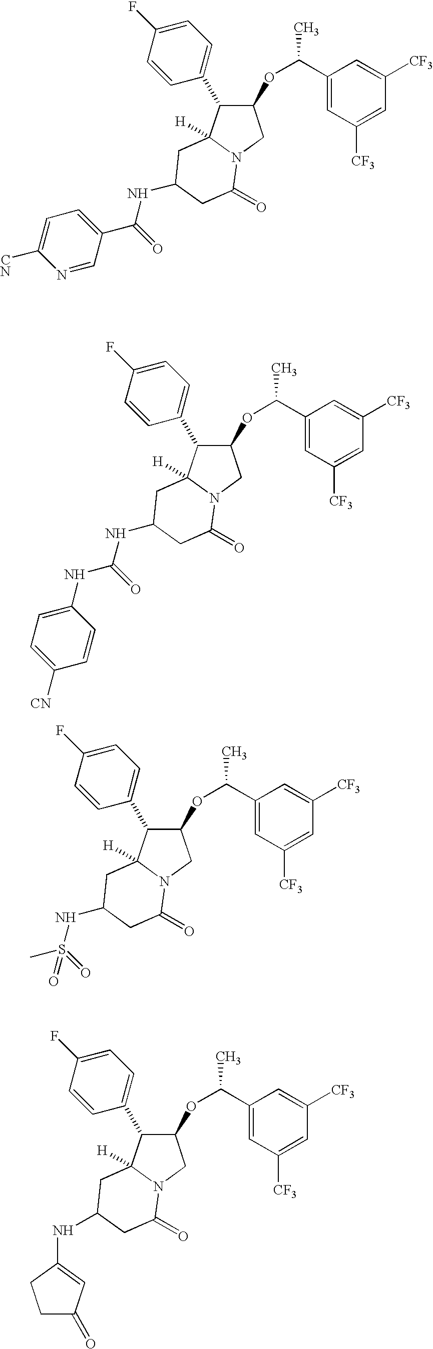 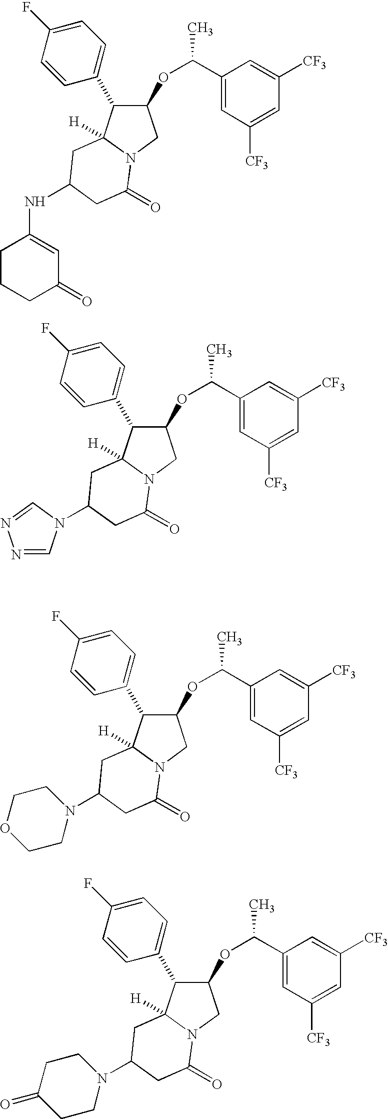 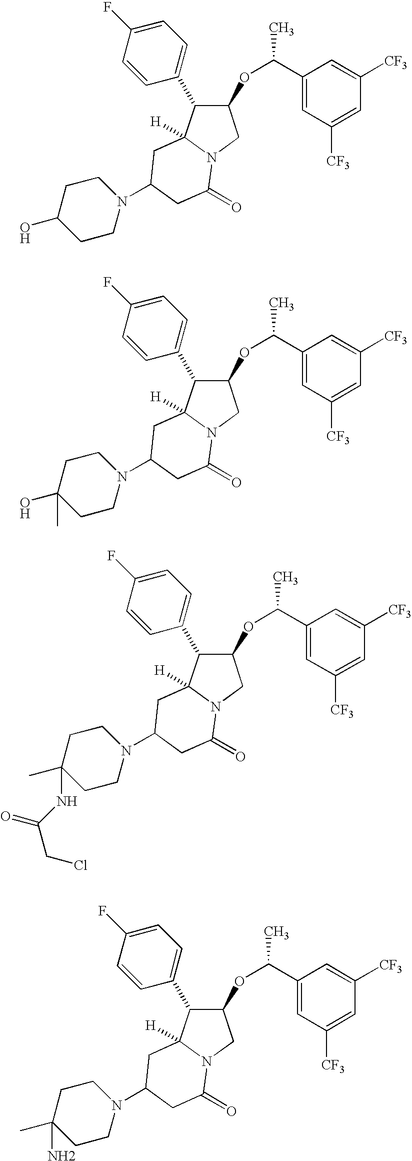 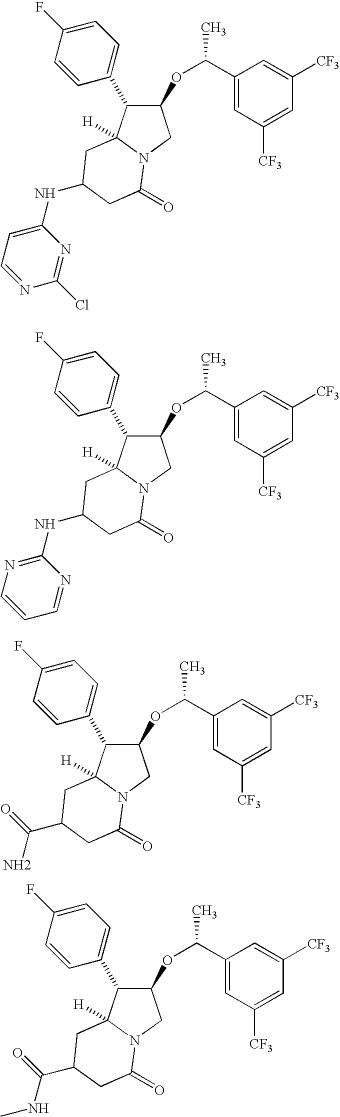 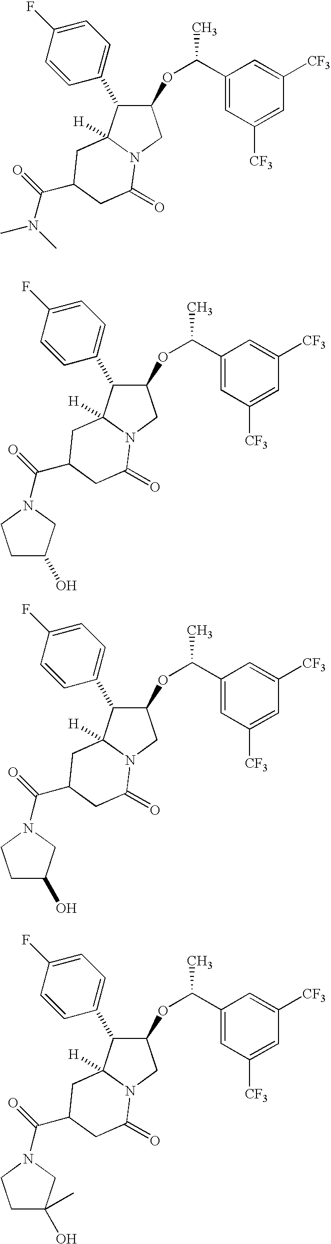 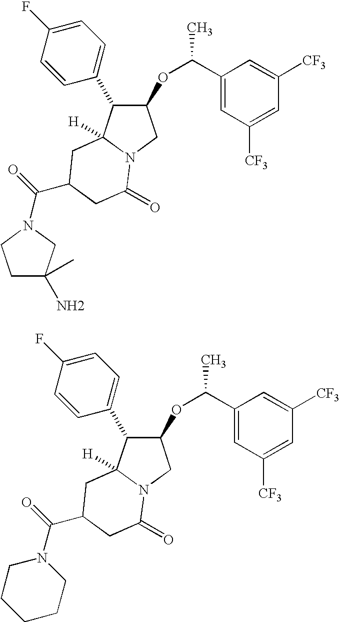 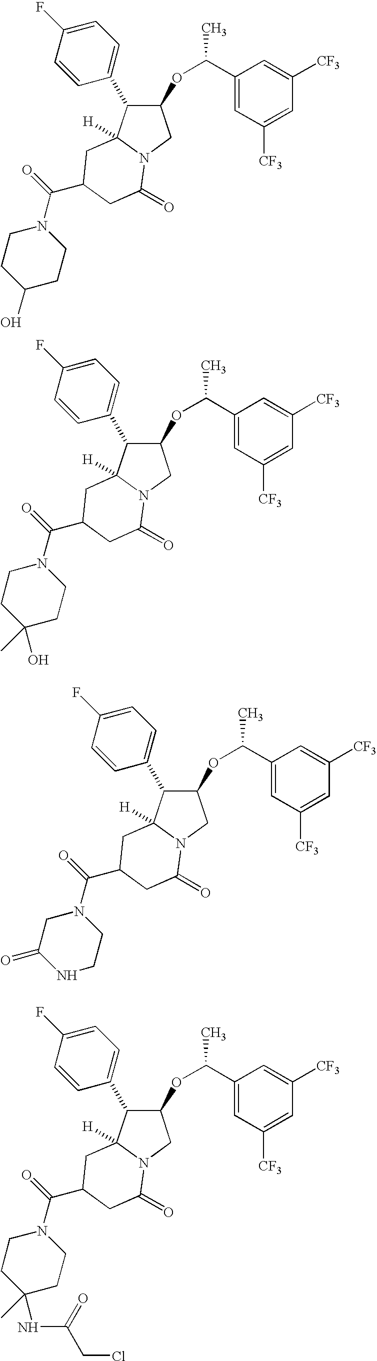 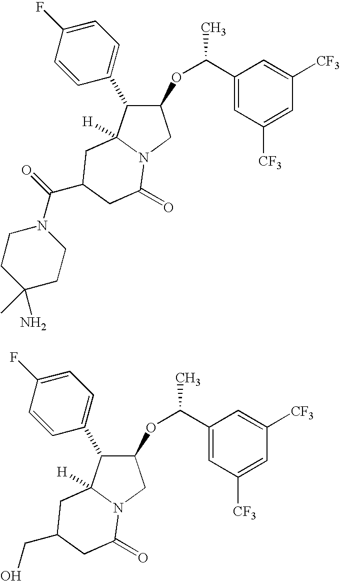 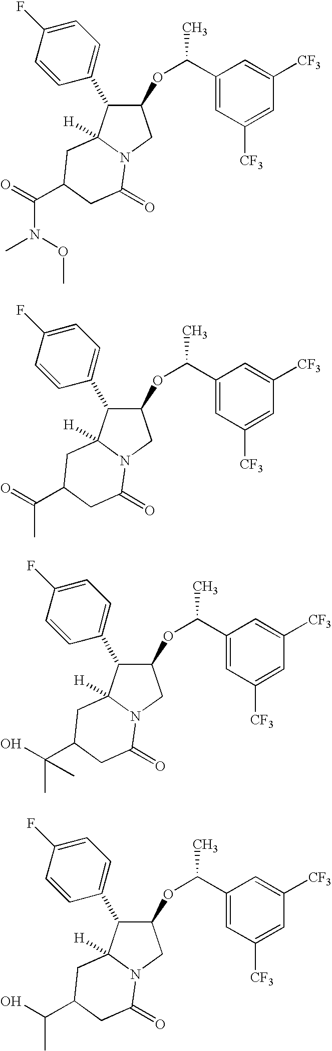 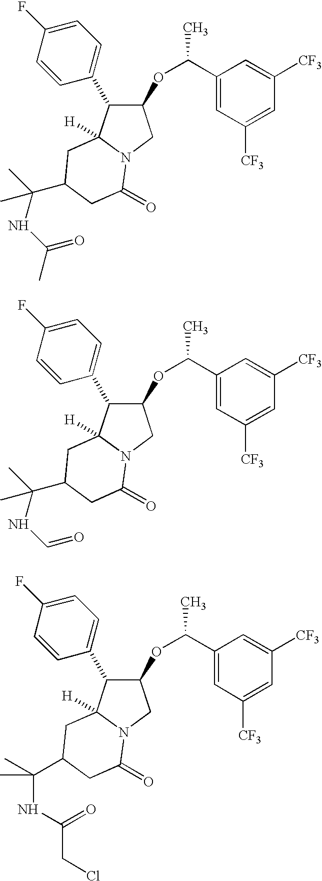 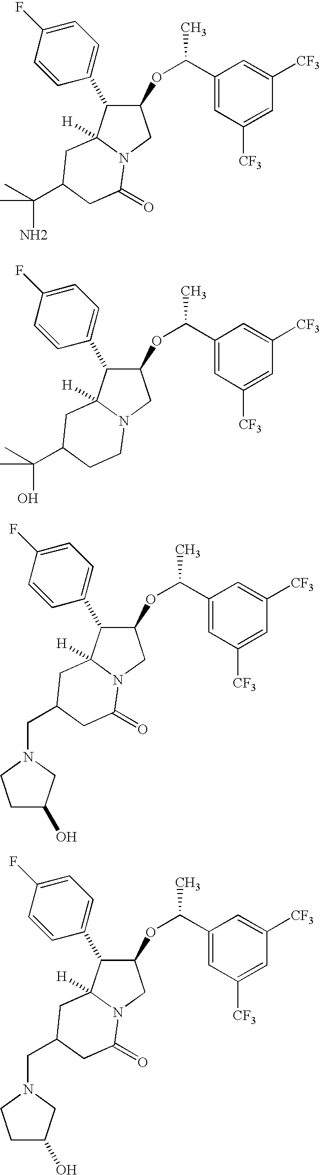 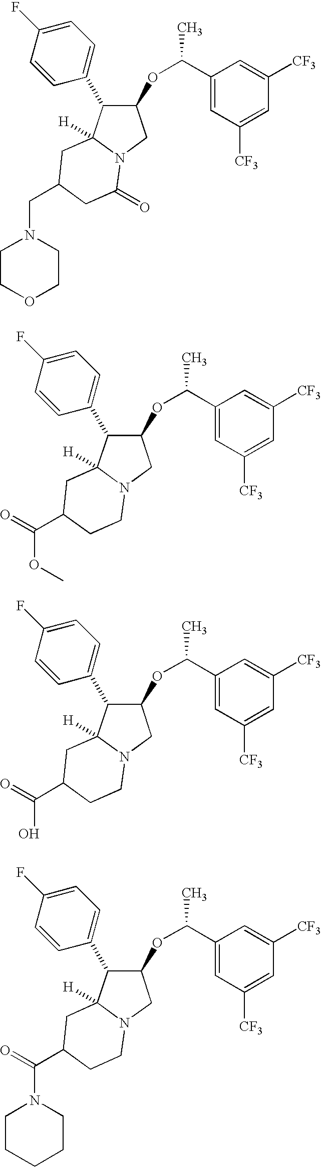 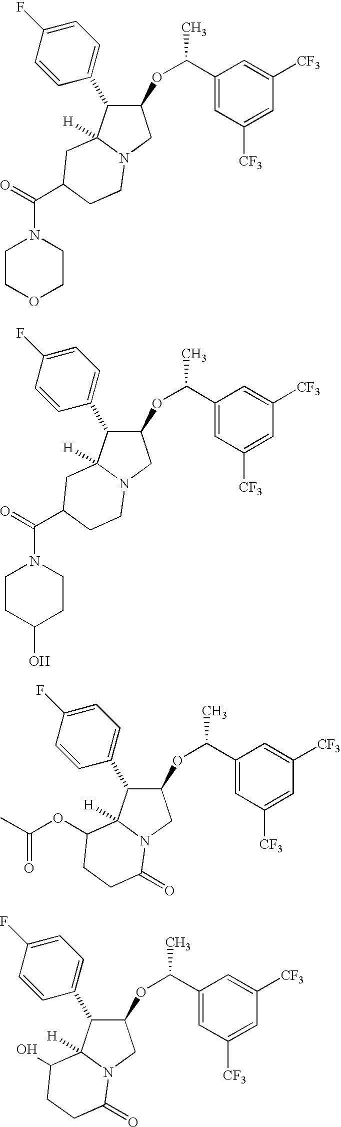 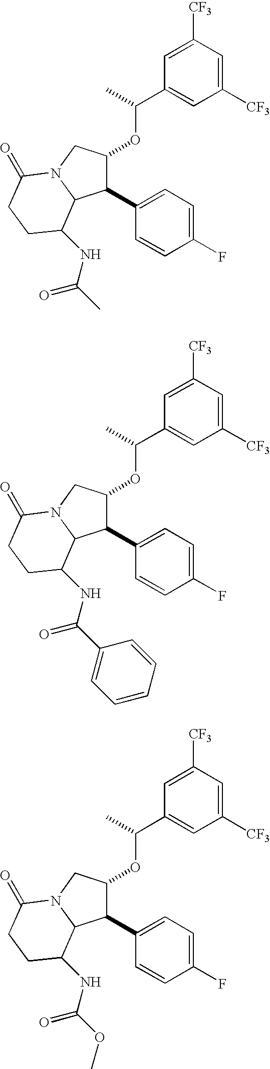 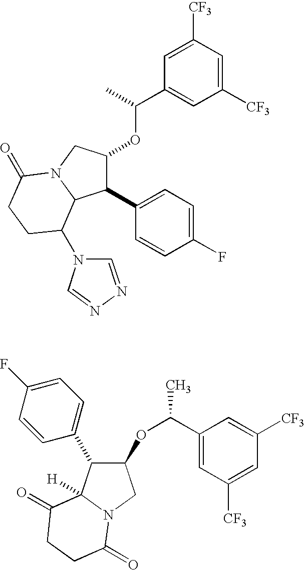 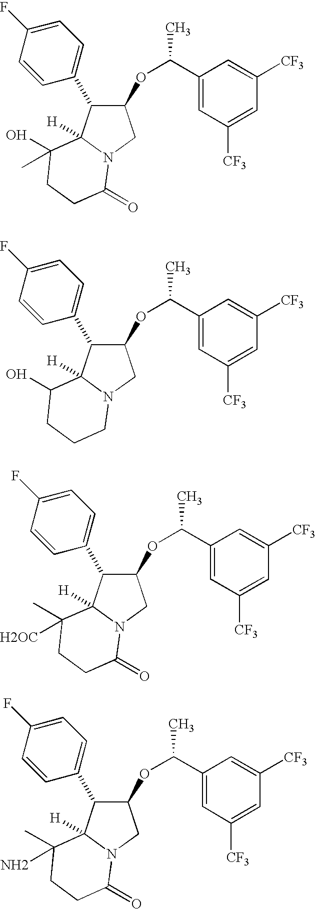 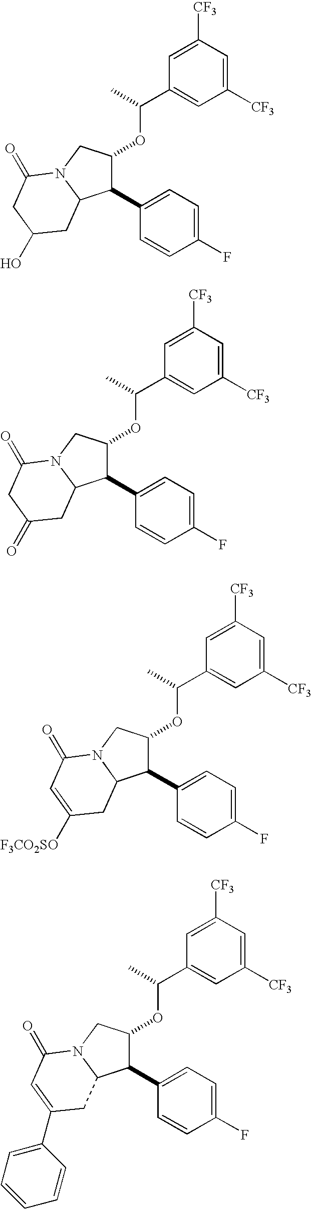 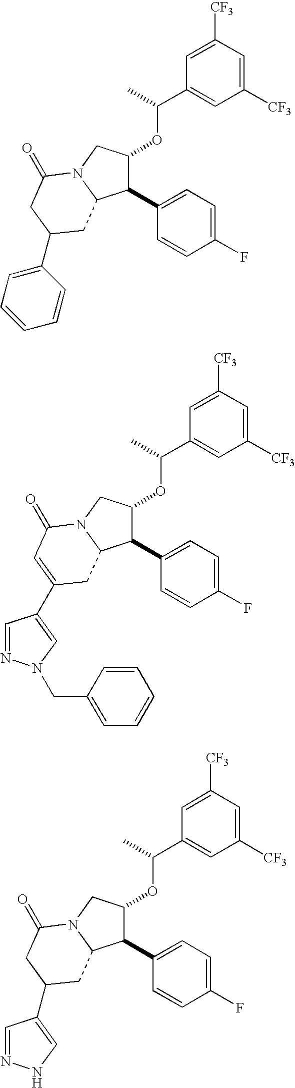 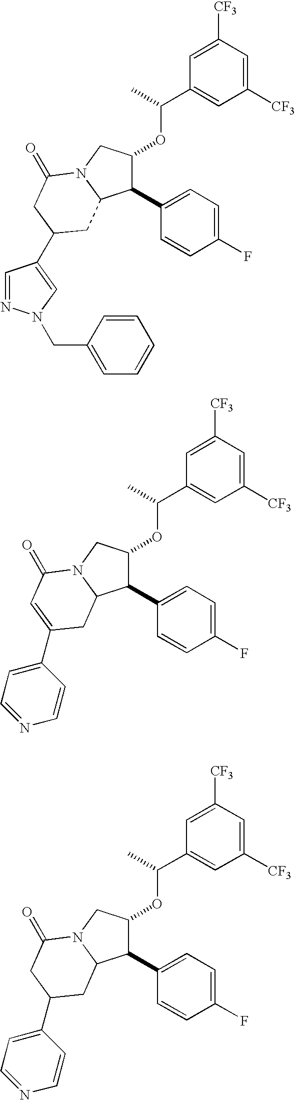 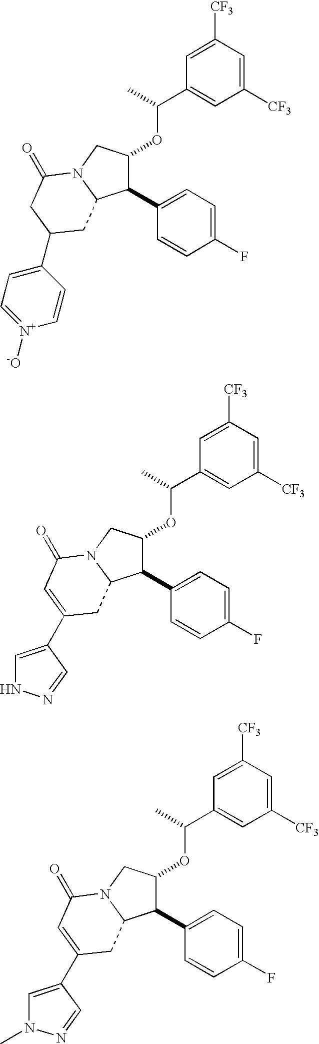 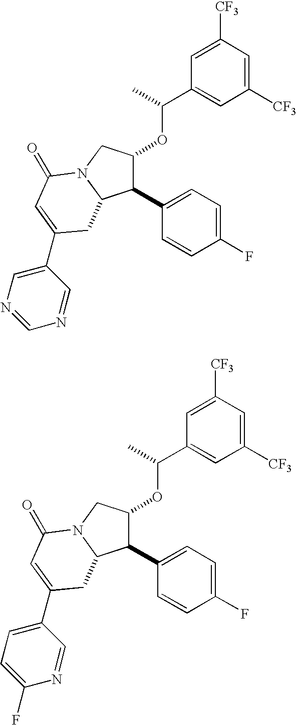 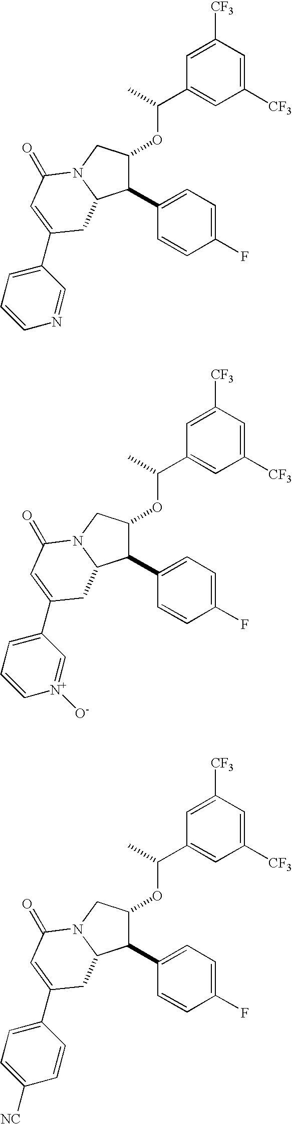 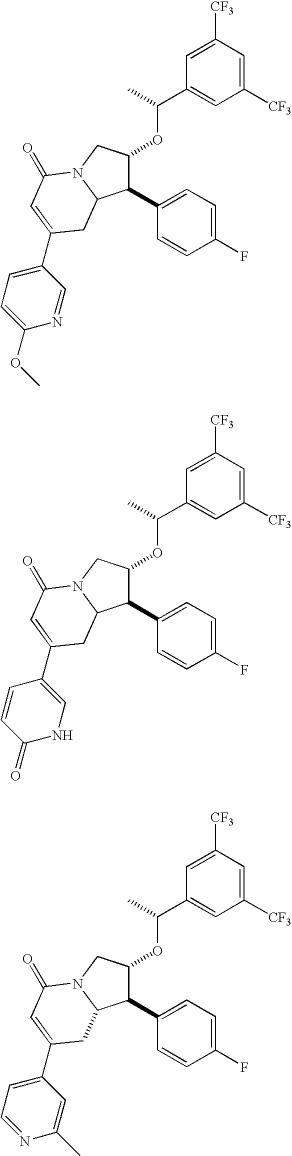 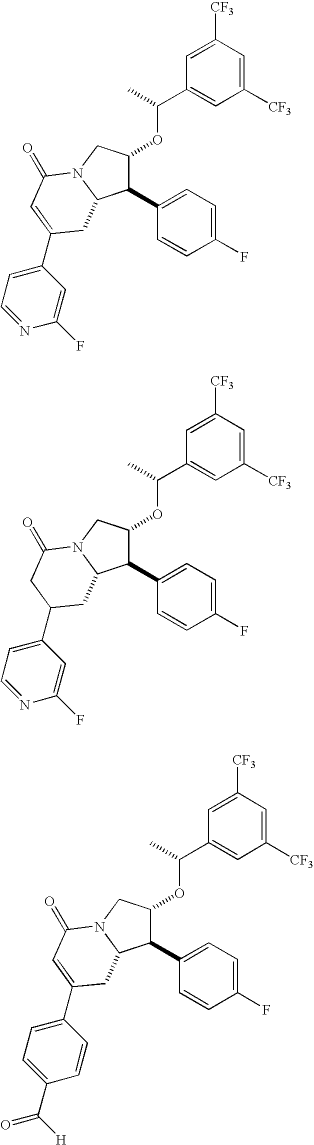 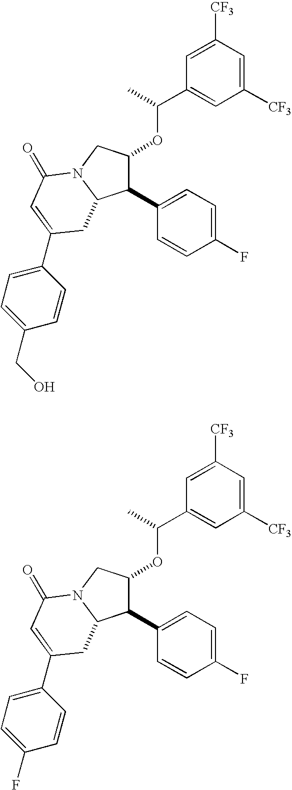 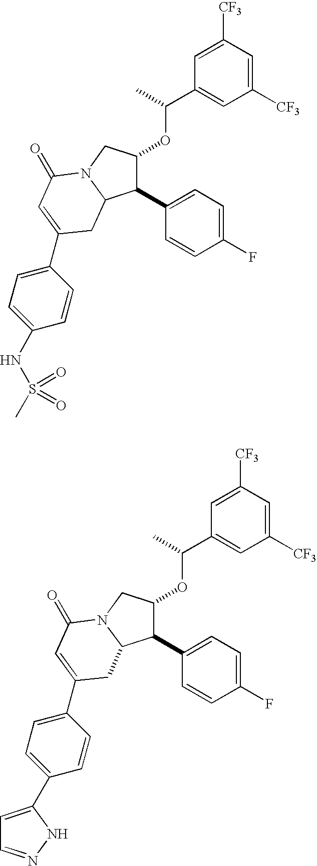 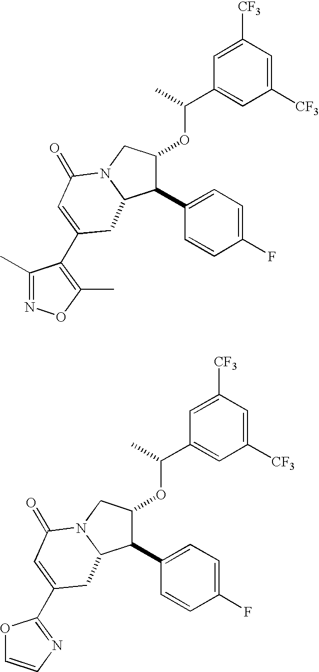 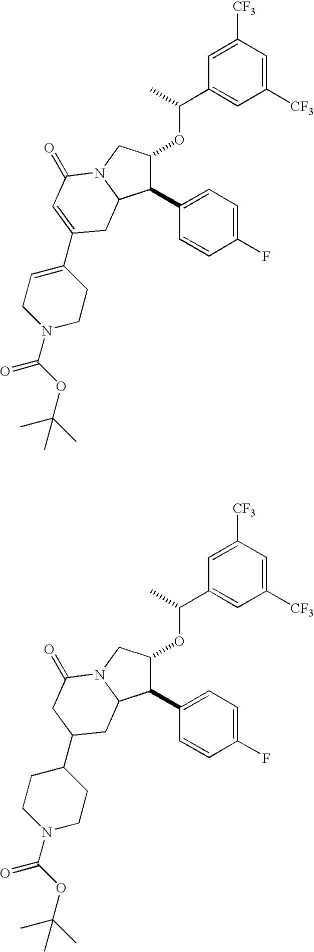 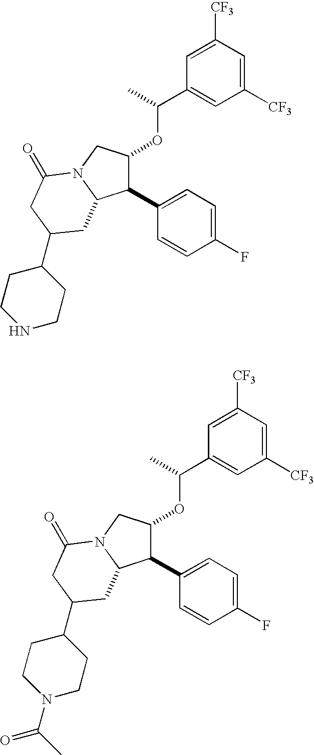 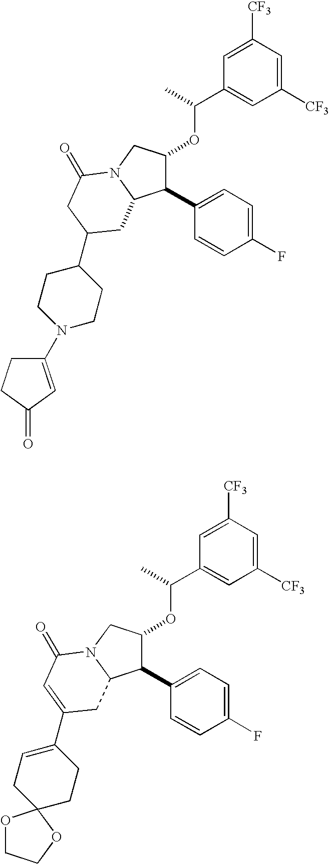 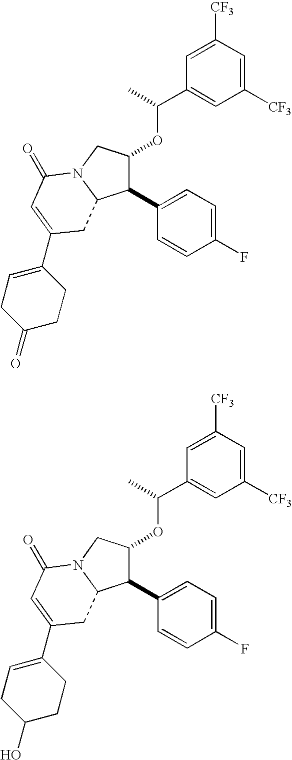 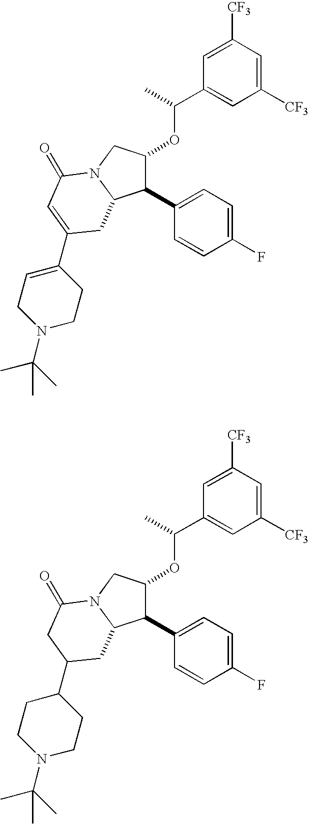 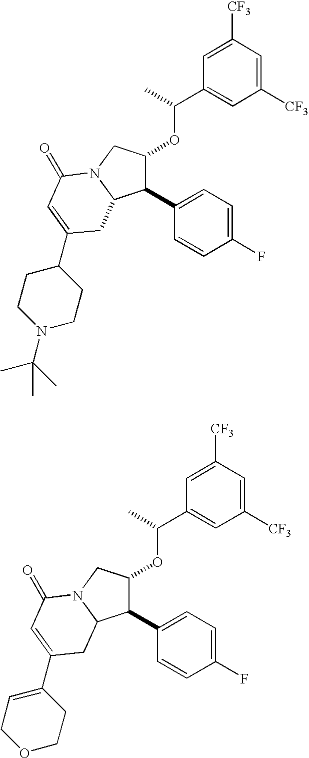 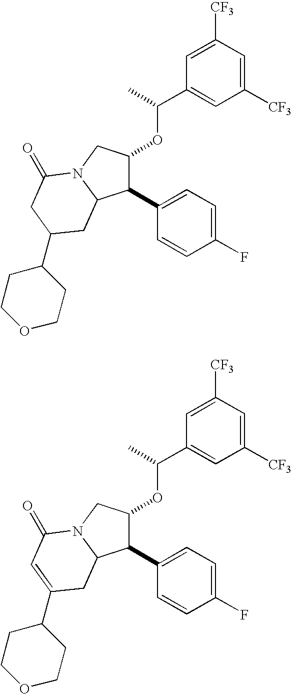 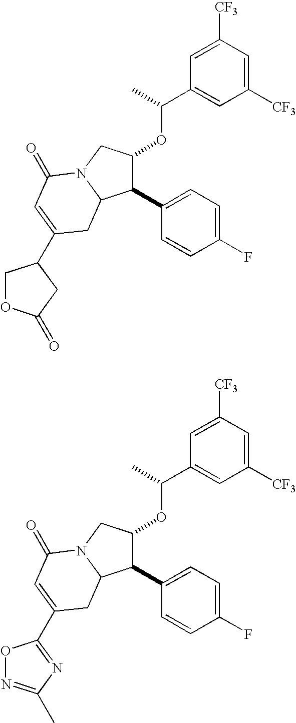 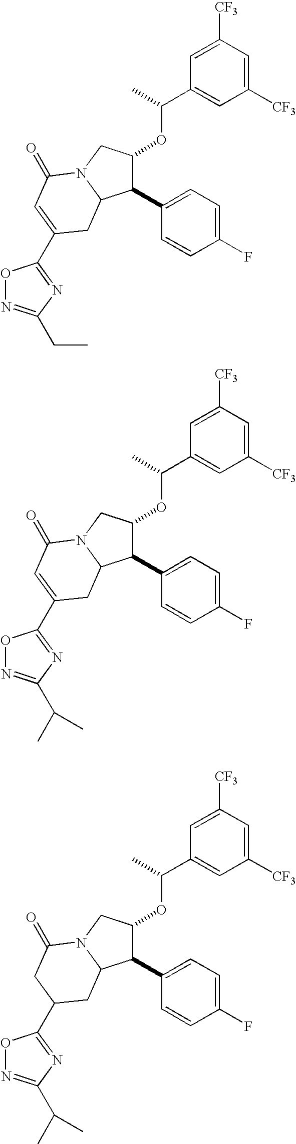 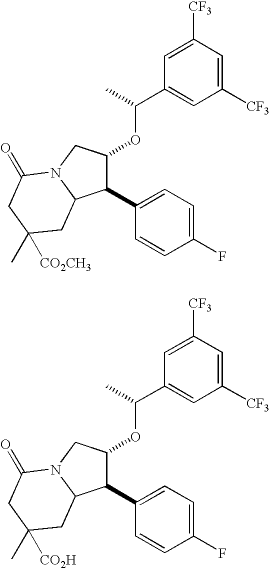 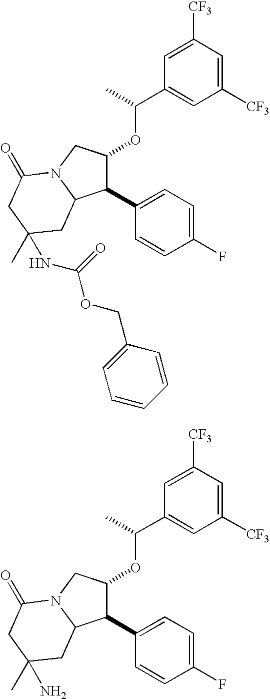 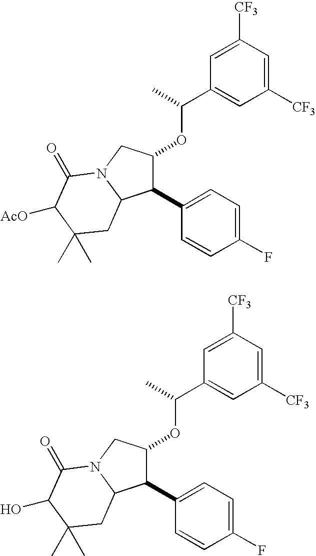
or a pharmaceutically acceptable salt thereof.

14. A pharmaceutical composition which comprises an inert carrier and a compound of claim 1 or a pharmaceutically acceptable salt thereof.

15. A method for the manufacture of a medicament for antagonizing the effect of substance P at its receptor site or for the blockade of neurokinin-1 receptors in a mammal comprising combining a compound of claim 1 or a pharmaceutically acceptable salt thereof with a pharmaceutical carrier or diluent.

16. A method for the manufacture of a medicament for the treatment of a physiological disorder associated with an excess of tachykinins in a mammal comprising combining a compound of claim 1 or a pharmaceutically acceptable salt thereof with a pharmaceutical carrier or diluent.
